(19) United States
(12) Patent Application Publication
(10) Pub. No.: US 2025/0280861 A1
(43) Pub. Date: Sep. 11, 2025

(54) SWEETENER COMPOSITIONS

(71) Applicant: Firmenich Incorporated, Plainsboro, NJ (US)

(72) Inventors: Joseph R. Fotsing, San Diego, CA (US); Andrew P. Patron, San Diego, CA (US); Vincent Darmohusodo, San Diego, CA (US); Melissa S. Wong, San Diego, CA (US); Chad Priest, San Diego, CA (US); Qing Chen, San Diego, CA (US); Guy Servant, San Diego, CA (US); Catherine Tachdjian, San Diego, CA (US); Sara L. Adamski-Werner, San Diego, CA (US)

(21) Appl. No.: 18/843,018

(22) PCT Filed: Mar. 1, 2023
(86) PCT No.: PCT/US2023/014224

§ 371 Date: Aug. 30, 2024

Related U.S. Application Data
(60) Provisional application No. 63/317,382, filed on Mar. 7, 2022.

(51) Int. Cl. A23L 27/20; A23L 27/00; C07D 211/14; C07D 211/18; C07D 211/22; C07D 211/26; C07D 211/34; C07D 211/64; C07D 211/70; C07D 213/74; C07D 217/04; C07D 223/04; C07D 241/20; C07D 295/145; C07D 295/155; C07D 401/04; C07D 405/04; C07D 491/107

(52) U.S. Cl. CPC A23L 27/2054; A23L 27/88; C07D 211/14; C07D 211/18; C07D 211/22; C07D 211/26; C07D 211/34; C07D 211/64; C07D 211/70; C07D 213/74; C07D 217/04; C07D 223/04; C07D 241/20; C07D 295/145; C07D 295/155; C07D 401/04; C07D 405/04; C07D 491/107

(57) ABSTRACT

Described herein are compounds useful as sweet flavor modifiers. Ingestible compositions that include one or more of these compounds in combination with a natural or artificial sweetener are also described. (I)

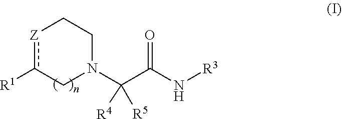

### CROSS-REFERENCE TO RELATED APPLICATIONS

This application claims benefit of U.S. Provisional Application No. 63/317,382 filed on Mar. 7, 2022, the contents of which are hereby incorporated by reference in their entirety for all purposes.

### BACKGROUND

### Field

The present disclosure relates to the fields of chemistry and flavor modifying compounds and sweeteners in foods, beverages, animal feed, pharmaceuticals, and other ingestible compositions. More specifically, the present disclosure relates to product combinations that include one or more sweet-tasting compounds and one or more flavor modifying compounds.

### Background Description

The taste system provides sensory information about the chemical composition of the external world. Taste transduction is one of the most sophisticated forms of chemical-triggered sensation in animals. Signaling of taste is found throughout the animal kingdom, from simple metazoans to the most complex of vertebrates. Mammals are believed to have five basic taste modalities: sweet, bitter, sour, salty, and umami (the taste of monosodium glutamate, a.k.a. savory taste).

Obesity, diabetes, and cardiovascular disease are health concerns on the rise globally, but are growing at alarming rates in the United States. Sugar and calories are key components that can be limited to render a positive nutritional effect on health. High-intensity sweeteners can provide the sweetness of sugar, with various taste qualities. Because they are many times sweeter than sugar, much less of the sweetener is required to replace the sugar.

High-intensity sweeteners have a wide range of chemically distinct structures and hence possess varying properties, such as, without limitation, odor, flavor, mouthfeel, and aftertaste. These properties, particularly flavor and aftertaste, are well known to vary over the time of tasting, such that each temporal profile is sweetener-specific.

Sweeteners such as saccharin and 6-methyl-1,2,3-oxathiazin-4(3H)-one-2,2-dioxide potassium salt (acesulfame potassium) are commonly characterized as having bitter and/or metallic aftertastes. Products prepared with 2,4-dihydroxybenzoic acid are claimed to display reduced undesirable aftertastes associated with sweeteners, and do so at concentrations below those concentrations at which their own tastes are perceptible. Also, high intensity sweeteners such as sucralose and aspartame are reported to have sweetness delivery problems, i.e., delayed onset and lingering of sweetness.

here is a need for new compounds that can enhance the sweetness of sweeteners.

### SUMMARY

Some embodiments herein relate to a compound having the structure of formula (I):

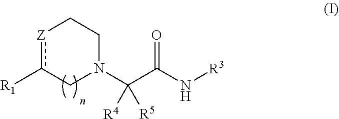

or a salt or stereoisomer thereof, wherein: [symbol] indicates a single or double bond; when [symbol] is a single bond, then Z is CR⁶R⁷ or NR⁸; when [symbol] is a double bond, then Z is CR⁶.

In some embodiments, R⁶ is an aryl optionally substituted with one or more R⁹, a heteroaryl optionally substituted with one or more R⁹, or a heterocyclyl optionally substituted with one or more R⁹; R⁷ is hydrogen, cyano, OR¹⁰, or C₁₋₆ alkyl optionally substituted with one or more R⁹; or alternatively, R⁶ and R⁷, taken together with the atom to which they are attached, form a heterocyclyl optionally substituted with one or more R⁹; R¹ is hydrogen, or alternatively, R¹ and R⁶, taken together with the atoms to which they are attached, form a fused aromatic ring and R⁷ is absent; R³ is C₁₋₆ alkyl optionally substituted with one or more R⁹, cycloalkyl, aralkyl, or (carbocyclyl)alkyl.

In some embodiments, R⁴ and R⁵ are each independently hydrogen, or a C₁₋₆ alkyl substituted with one or more R⁹; or alternatively, R⁴ and R⁵, taken together with the atom to which they are attached, form a heterocyclyl optionally substituted with one or more R⁹; R⁸ is an aryl optionally substituted with one or more R⁹, or a heteroaryl optionally substituted with one or more R⁹.

In some embodiments, n is 1 or 2; each R⁹ is independently halogen, C-carboxy, C-amido, cycloalkyl, OR¹⁰, cyano, amino, aminoalkyl, C₁-C₆ alkoxy, or C₁₋₆ alkyl optionally substituted with one or more OR¹⁰, C₁-C₆ alkoxy, phenyl, or halogen; or alternatively, two R⁹ groups taken together with the atom(s) to which they are attached, form a heterocyclyl or aryl; and R¹⁰ is hydrogen or C₁₋₆ alkyl.

In some embodiments, n is 1. In some embodiments, n is 2.

In some embodiments, R¹ is hydrogen. In some embodiments, R¹ and R⁶ are taken together with the atoms to which they are attached to form an aromatic ring.

In some embodiments, Z is CR⁶R⁷. In some embodiments, Z is NRs.

Some embodiments relate to a compound having the structure of formula (Ia):

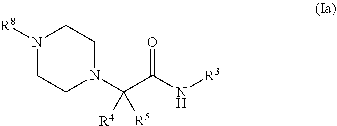

or a salt or stereoisomer thereof.

In some embodiments, R⁸ is an aryl optionally substituted with one or more R⁹. In some embodiments, R⁸ is phenyl. In some embodiments, R⁸ is a heteroaryl optionally substituted with one or more R⁹. In some embodiments, R⁸ is pyridinyl optionally substituted with one or more R⁹. In some embodiments, R⁸ is pyrazinyl optionally substituted with one or more R⁹.

Some embodiments relate to a compound having the structure of formula (Ib):

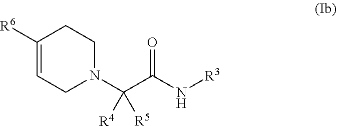

or a salt or stereoisomer thereof.

In some embodiments, R⁶ is aryl optionally substituted with one or more R⁹. In some embodiments, R⁶ is phenyl optionally substituted with one or more R⁹. In some embodiments, R⁶ is heteroaryl optionally substituted with one or more R⁹. In some embodiments, R⁶ is pyridinyl optionally substituted with one or more R⁹. In some embodiments, R⁶ is a heterocyclyl optionally substituted with one or more R⁹. In some embodiments, R⁶ is 1,3-benzodioxolyl optionally substituted with one or more R⁹.

Some embodiments relate to a compound having the structure of formula (Ic):

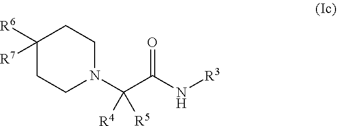

or a salt or stereoisomer thereof.

In some embodiments, R⁶ is an aryl optionally substituted with one or more R⁹. In some embodiments, R⁶ is phenyl optionally substituted with one or more R⁹. In some embodiments, R⁶ is a heteroaryl optionally substituted with one or more R⁹. In some embodiments, R⁶ is pyridinyl optionally substituted with one or more R⁹. In some embodiments, R⁶ is a heterocyclyl optionally substituted with one or more R⁹. In some embodiments, R⁶ is 1,3-benzodioxolyl optionally substituted with one or more R⁹.

In some embodiments, R⁷ is hydrogen. In some embodiments, R⁷ is cyano. In some embodiments, R⁷ is OR¹⁰. In some embodiments, R¹⁰ is hydrogen. In some embodiments, R⁷ is C₁₋₆ alkyl substituted with one or more R⁹.

Some embodiments relate to a compound having the structure of formula (Id):

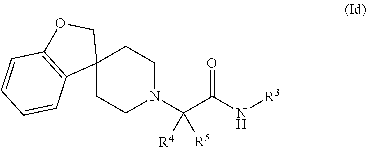

or a salt or stereoisomer thereof.

Some embodiments relate to a compound having the structure of formula (Ie):

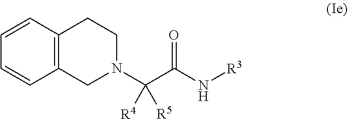

or a salt or stereoisomer thereof.

Some embodiments relate to a compound having the structure of formula (If):

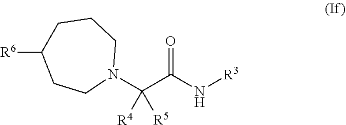

or a salt or stereoisomer thereof.

In some embodiments, R⁶ is aryl optionally substituted with one or more R⁹. In some embodiments, R⁶ is phenyl optionally substituted with one or more R⁹.

In some embodiments, R³ is C₁₋₆ alkyl optionally substituted with one or more R⁹. In some embodiments, R³ is iso-butyl optionally substituted with one or more R⁹. In some embodiments, R³ is ethyl optionally substituted with one or more R⁹. In some embodiments, R³ is propyl optionally substituted with one or more R⁹. In some embodiments, R³ is neopentyl optionally substituted with one or more R⁹. In some embodiments, R³ is alkyl substituted with one or more OR¹⁰ or amino. In some embodiments, R³ is cycloalkyl. In some embodiments, R³ is cyclopropyl. In some embodiments, R³ is cyclobutyl. In some embodiments, R³ is cyclopentyl. In some embodiments, R³ is cyclohexyl. In some embodiments, R³ is aralkyl. In some embodiments, R³ is (carbocyclyl)alkyl.

In some embodiments, R⁴ is hydrogen. In some embodiments, R⁴ is C₁₋₆ alkyl optionally substituted with one or more R⁹. In some embodiments, R⁴ is methyl optionally substituted with one or more R⁹.

In some embodiments, R⁵ is hydrogen. In some embodiments, R⁵ is C₁₋₆ alkyl optionally substituted with one or more R⁹. In some embodiments, R⁵ is methyl optionally substituted with one or more R⁹. In some embodiments, R⁵ is alkyl substituted with one or more OR¹⁰.

In some embodiments, R⁴ and R⁵, taken together with the atom to which they are attached, form a heterocyclyl optionally substituted with one or more R⁹.

Some embodiments relate to a compound selected from the group consisting of:

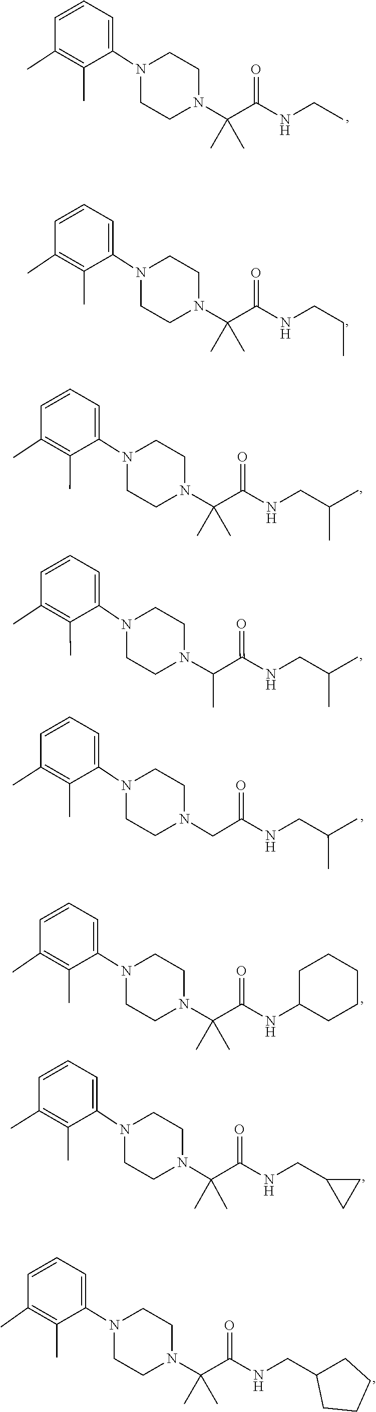

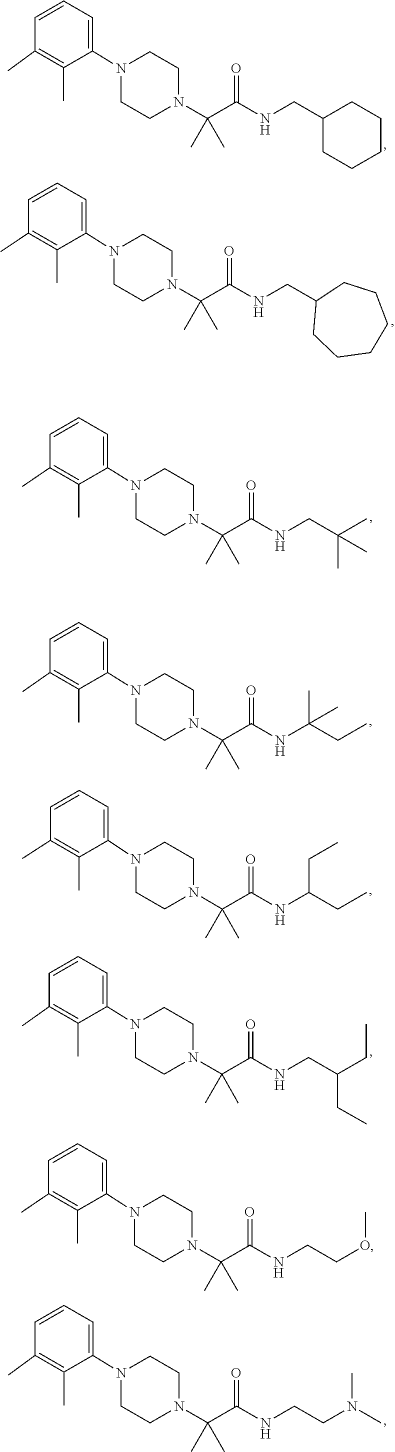

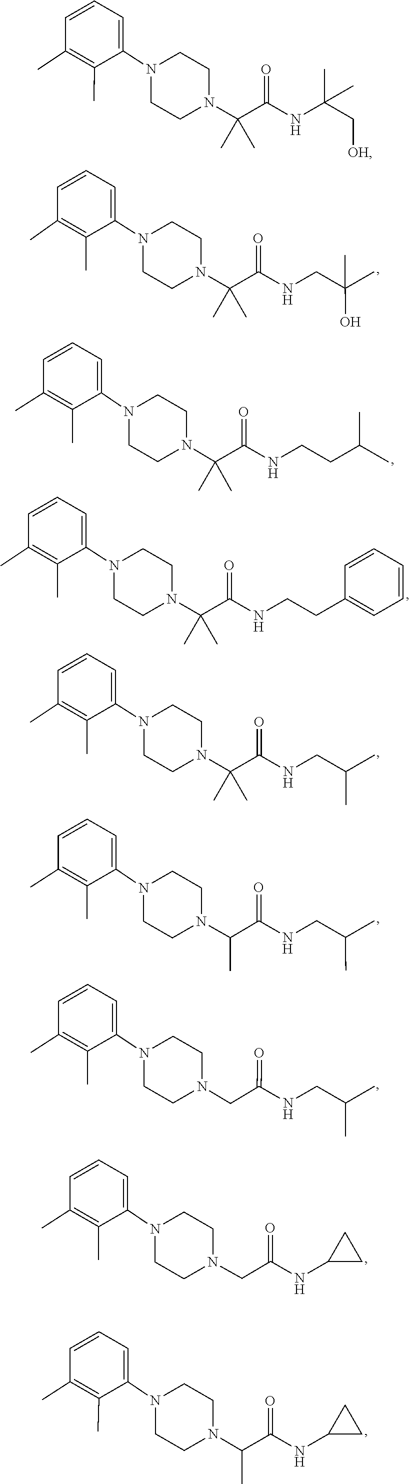

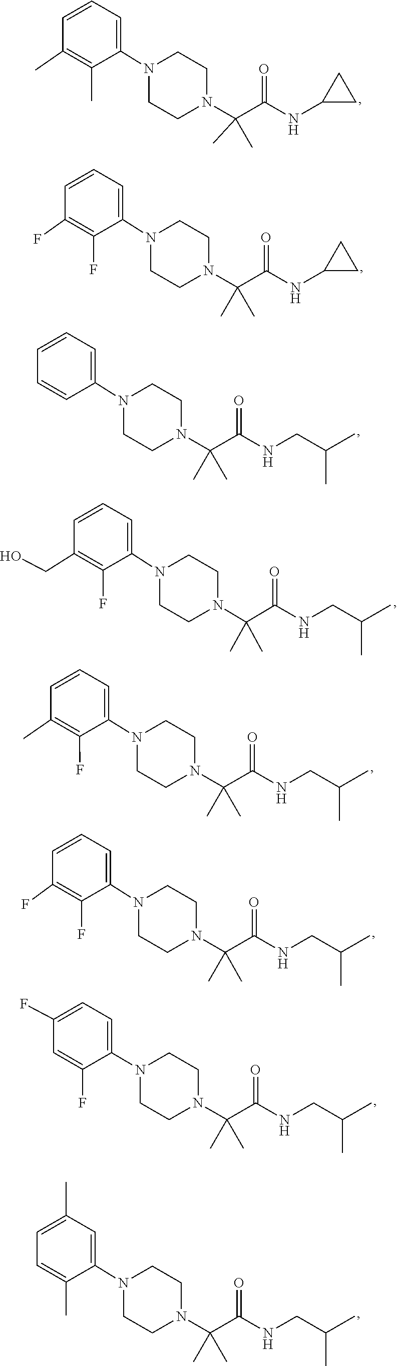

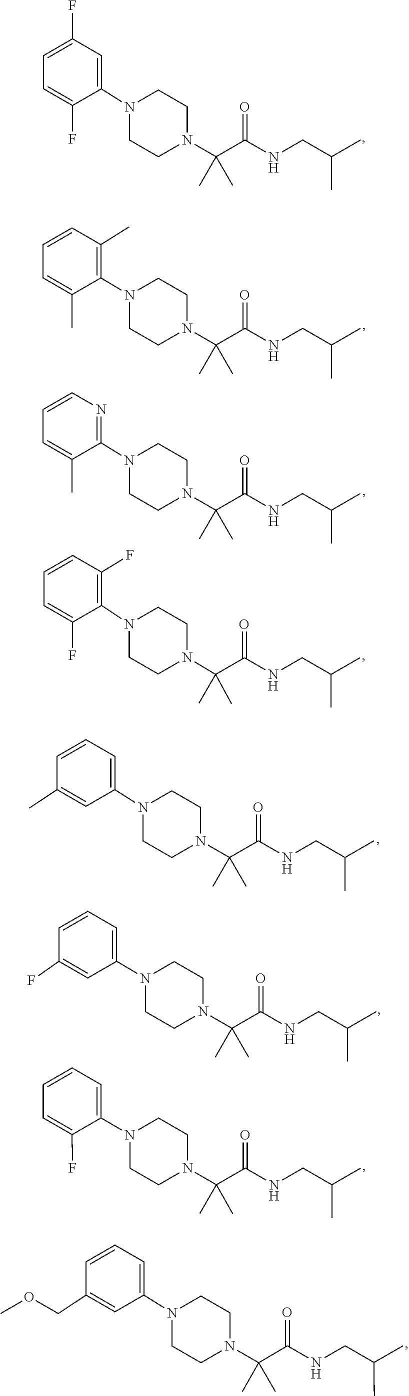

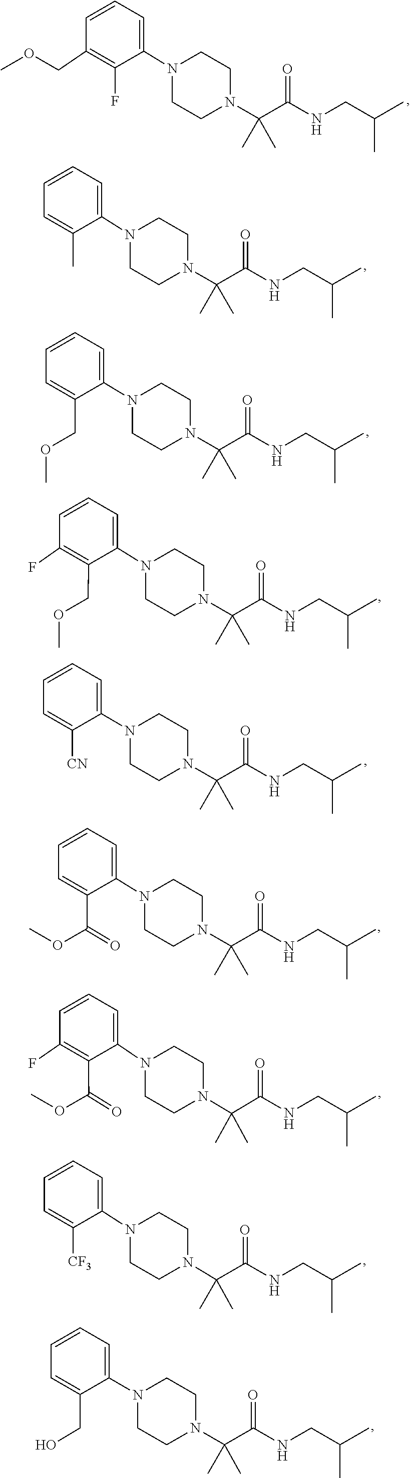

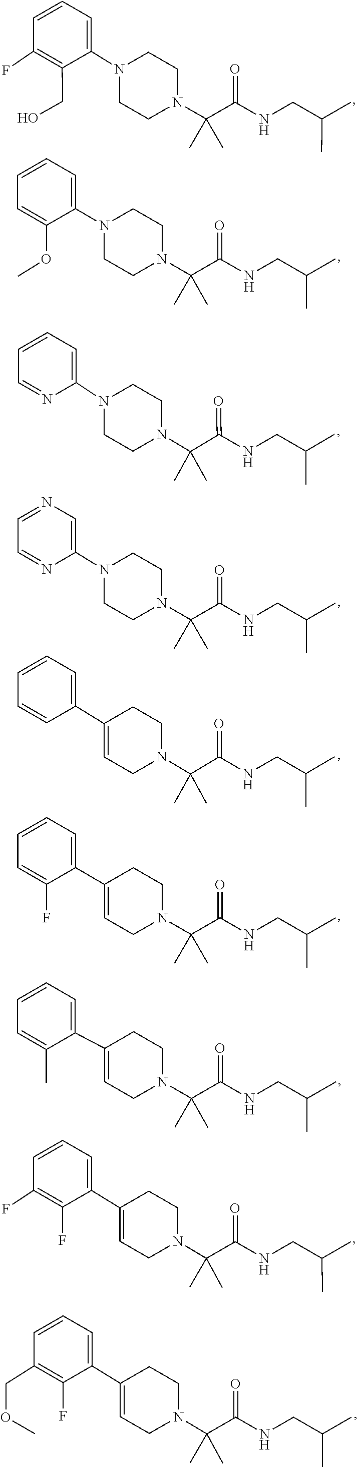

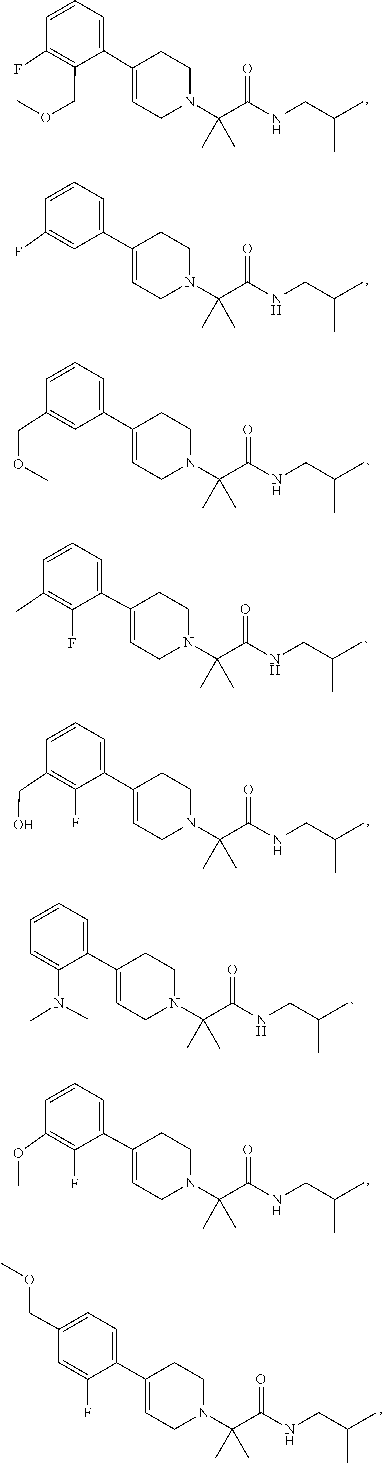

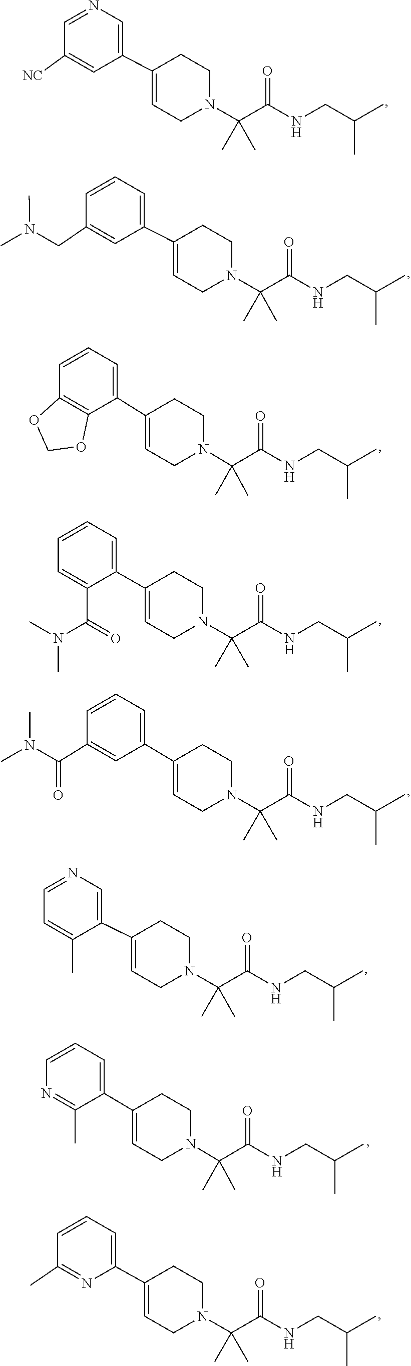

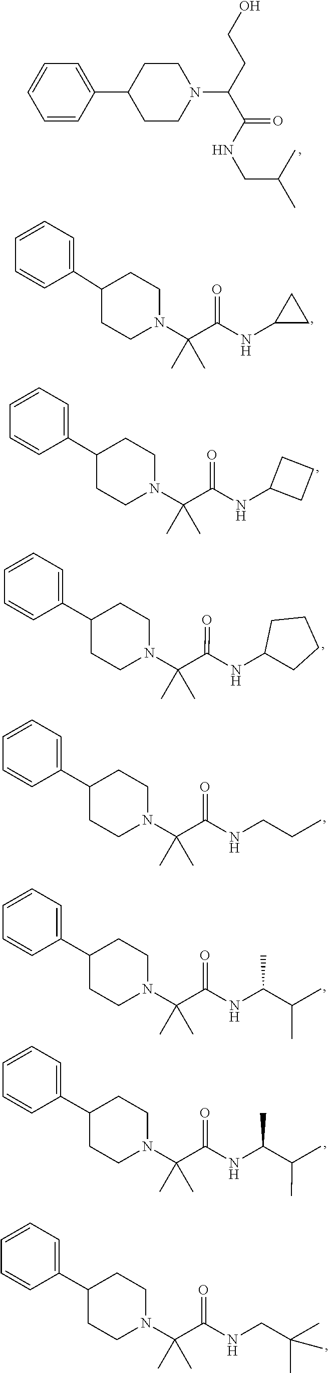

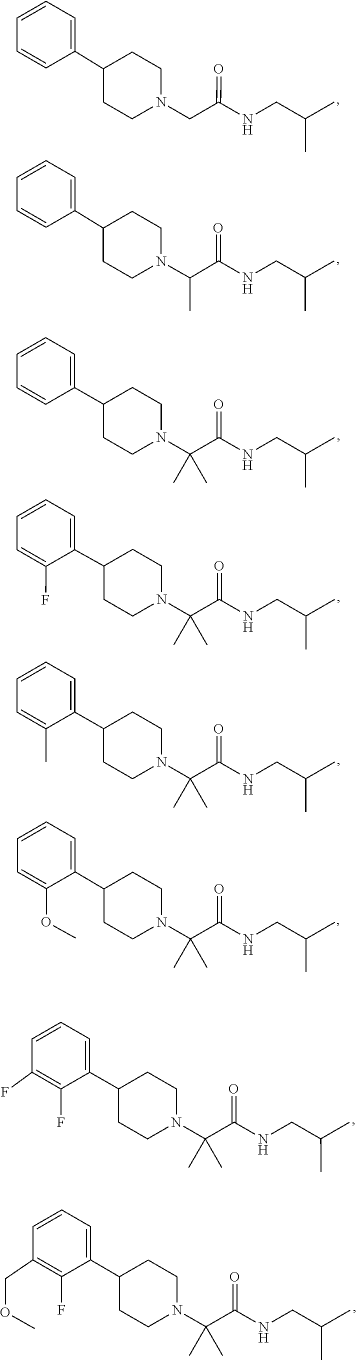

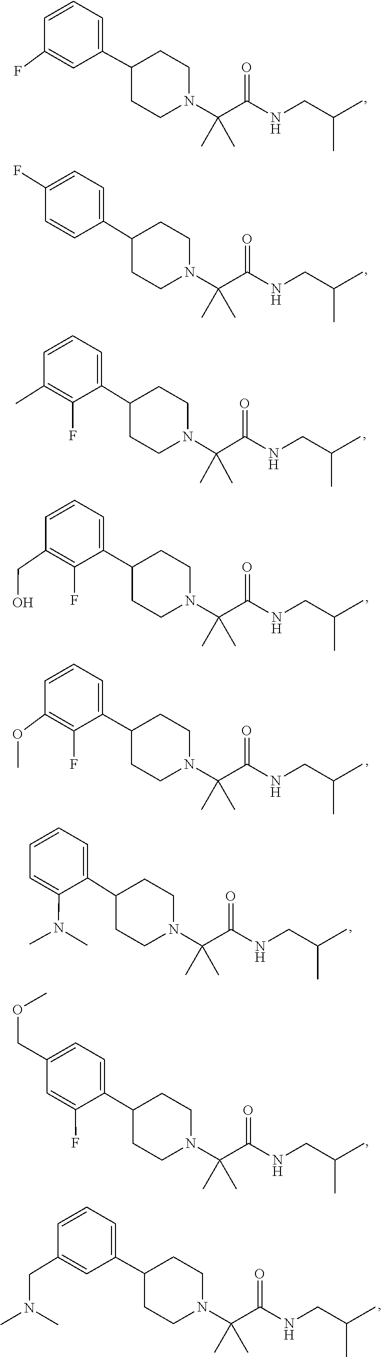

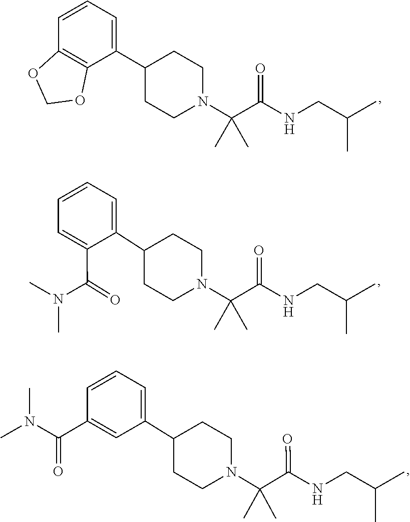

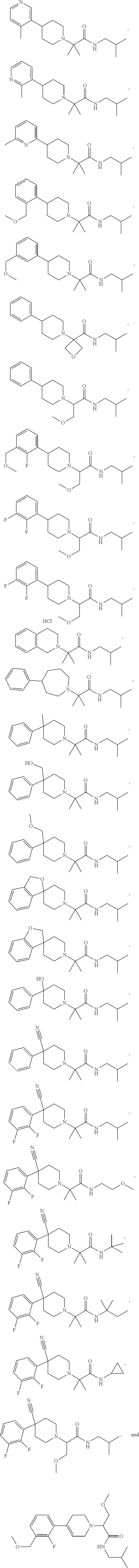

and or a salt or stereoisomer thereof.

Some embodiments relate to a n ingestible composition comprising a compound as described herein and one or more sweeteners. In some embodiments, the sweetener is sugar. In some embodiments, the sweetener is sucrose. In some embodiments, the sweetener comprises a combination of fructose and glucose. In some embodiments, the sweetener is sucralose. In some embodiments, the sweetener is high fructose corn syrup.

Some embodiments relate to a method of enhancing sweetness of a sweetener, comprising combining a compound as described herein with a sweetener. In some embodiments, the sweetener is sugar. In some embodiments, the sweetener is sucrose. In some embodiments, the sweetener comprises a combination of fructose and glucose. In some embodiments, the sweetener is sucralose. In some embodiments, the sweetener is high fructose corn syrup.

Some embodiments relate to a compound as described herein for use in enhancing the sweetness of a sweetener.

Some embodiments relate to use of a compound described herein for enhancing the sweetness of a sweetener.

In addition to the features described above, additional features and variations will be readily apparent from the following description. It is to be understood that the following description describes typical alternatives, and is not intended to be limiting in scope. Although this disclosure is described in various exemplary alternatives and implementation as provided herein, it should be understood that the various features, aspects, and functionality described in one or more of the individual alternatives are not limited in their applicability to the particular alternative with which they are described. Instead, they can be applied alone or in various combinations to one or more of the other alternatives, whether the alternatives are described or whether the features are presented as being part of the described alternative. The breadth and scope of the present disclosure should therefore not be limited by any exemplary alternatives described herein.

### BRIEF DESCRIPTION OF THE DRAWINGS

FIG. 1 is a graph depicting the dose-dependent hT1R2/T1R3 agonist activity of compound 114.

### DETAILED DESCRIPTION

Embodiments disclosed herein relate generally to flavor modifying compounds. In some embodiments, the flavor modifying compounds are sweetener enhancers. Some embodiments include compositions that include the flavor modifying compounds and one or more sweeteners. In some embodiments, these compositions comprise non-caloric or low-caloric high-potency natural sweeteners. In some embodiments, the composition comprises the combination of one or more sweetener and one or more flavor modifying compound that can activate the sweet receptor in vitro and impart a sweet taste enhancement. The combination composition can be used in a variety of ingestible or non-ingestible compositions. In some embodiments, the ingestible composition comprises one or more flavor modifying compound and one or more sweeteners, which can be a natural sweetener, e.g., sucrose; or a synthetic sweetener, e.g., sucralose. In some embodiments, the natural or synthetic sweetener is a high potency sweetener. The present disclosure also relates to compositions that can improve the tastes of non-caloric or low-caloric natural and/or synthetic, high-potency sweeteners by imparting a more sugar-like taste or characteristic by utilizing natural sweeteners in conjunction with other natural or synthetic sweeteners. In some embodiments, the ingestible compositions provide a more sugar-like temporal profile, including sweetness onset and sweetness linger, and/or a more sugar-like flavor profile.

In some embodiments, the ingestible composition can be food or beverage products.

In some embodiments, the beverage can be selected from enhanced sparkling beverages, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea-type drinks, coffees, cocoa drinks, beverages containing milk components, milk alternative beverages, beverages containing cereal extracts, and smoothies.

In some embodiments, the composition can be an animal feed product or animal feed ingredient.

In some embodiments, the ingestible composition can be pharmaceutical products, nutritional products, dietary supplements, or over-the-counter medications. In some embodiments, the non-ingestible composition can be oral care products, hygienic or cosmetic products.

These and other embodiments, advantages, and features of the present disclosure are provided in part in the description that follows, and in part will be obvious from the description, or may be learned by practice of the embodiments disclosed herein. It is to be understood that both the foregoing general description and the following detailed description are exemplary and explanatory only and are not restrictive of the embodiments as described.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated (e.g., where the stereochemistry of a chiral center is explicitly shown), all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

In some embodiments, the compounds disclosed herein are capable of forming acid and/or base salts by virtue of the presence of amino and/or carboxyl groups or groups similar thereto. Physiologically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Physiologically acceptable salts can be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

The terms “a” and “an” do not denote a limitation of quantity, but rather denote the presence of at least one of the referenced item. The term “or” or “and/or” is used as a function word to indicate that two words or expressions are to be taken together or individually. The terms “comprising”, “having”, “including”, and “containing” are to be construed as open-ended terms (for example, meaning “including, but not limited to”). The endpoints of all ranges directed to the same component or property are inclusive and independently combinable.

Many types of plants are known for producing sweet tasting compounds and/or flavor modifying compounds, and these compounds have been isolated and used as sweeteners and/or flavor modifying compounds. In some embodiments herein, the formulation of one or more sweetener in combination with one or more flavor modifying compound is obtained in whole or in part from a plant extract. By “extract”, it is meant a substance or mixture that has been taken from a plant by at least one purification or other processing step. The “plant”, as used herein, includes but is not limited to a whole plant, a plant part, a plant tissue, a plant cell, or a combination thereof. In some embodiments, the extract is obtained from a plant, a plant part, a plant tissue or a plant cell. As used herein, “plant part” or “plant tissue” refers to any part of a plant. Examples of plant parts include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, spores, stolon, shoot, pericarp, endosperm, placenta, berry, stamen, sap, and leaf sheath.

The term “isolated” when referring to a compound, as used herein, means separated or isolated away from other components, ingredients, or chemicals which co-exist with the compound of interest regardless whether the other components, ingredients, or chemicals are used or generated when chemically or enzymatically synthesizing the compound of interest, or the other components, ingredients, or chemicals exist with the compound of interest in nature in its native state. In some embodiments, the term “isolated’ means that the compound of interest is substantially or essentially freed from components, ingredients, or chemicals that normally accompany it in its native state by at least one purification or other processing step. Such an isolated compound may also be described as substantially pure. The term “substantially pure” as used herein describes a compound of interest that has been separated from components, ingredients, or chemicals that naturally accompany it. In some embodiments, an isolated compound is substantially pure when at least about 50%, at least about 60%, or at least about 70%, or at least about 80%, or at least about 90%, or at least about 95%, or at least about 96%, or at least about 97%, or at least about 98%, or at least about 99% of the total material (by volume, by wet or dry weight, or by mole percent or mole fraction) is the compound of interest. Purity can be measured by any appropriate method, for example by chromatography, gel electrophoresis, or HPLC analysis.

“Salt” refers to a salt of a compound, which possesses the desired activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Solvate” refers to the compound formed by the interaction of a solvent and a compound described herein or salt thereof. Suitable solvates are physiologically acceptable solvates including hydrates.

As used herein, “Ca to Cb” or “Ca-b” in which “a” and “b” are integers refer to the number of carbon atoms in the specified group. That is, the group can contain from “a” to “b”, inclusive, carbon atoms. Thus, for example, a “C₁ to C₄ alkyl” or “C₁_₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, isopropyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “substituted alkyl” refers to an alkyl group substituted with one or more substituents independently selected from C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, aryloxy (optionally substituted with halo, C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl(mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₃-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O).

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” refers to the formula —SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁_₄ heteroalkyl” indicates that there are one to four carbon atoms in the heteroalkyl chain and additionally one or more heteroatoms in the backbone of the chain.

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1,1-diyl, but-3-en-1,1-diyl, 1-methyl-prop-2-en-1,1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of 0, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-carbamyl” group refers to an “—N(R_{A})C(═O)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-thiocarbamyl” group refers to an “—N(R_{A})C(═S)OR_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocycyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocycyl (optionally substituted with halo, C₁-C₆ alkyl, C₁-C₆ alkoxy, C₁-C₆ haloalkyl, and C₁-C₆ haloalkoxy), 3-10 membered heterocycyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy (optionally substituted with halo, C₁-C₆ alkyl, C₁-C₆ alkoxy, C₁-C₆ haloalkyl, and C₁-C₆ haloalkoxy), C₃-C₇ carbocyclyloxy (optionally substituted with halo, C₁-C₆ alkyl, C₁-C₆ alkoxy, C₁-C₆ haloalkyl, and C₁-C₆ haloalkoxy), 3-10 membered heterocyclyl-oxy (optionally substituted with halo, C₁-C₆ alkyl, C₁-C₆ alkoxy, C₁-C₆ haloalkyl, and C₁-C₆ haloalkoxy), 5-10 membered heteroaryl-oxy (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkoxy (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkoxy (optionally substituted with halo, C₁-C₆ alkyl, C₁-C₆ alkoxy, C₁-C₆ haloalkyl, and C₁-C₆ haloalkoxy), aryl(C₁-C₆)alkoxy (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkoxy (optionally substituted with halo, C₁-C₆ alkyl, C₁-C₆ alkoxy, C₁-C₆ haloalkyl, and C₁-C₆ haloalkoxy), sulfhydryl (mercapto), halo(C₁-C₆)alkyl (e.g., —CF₃), halo(C₁-C₆)alkoxy (e.g., —OCF₃), C₁-C₆ alkylthio, arylthio (optionally substituted with halo, C₁-C₆ alkyl, C₁-C₆ alkoxy, C₁-C₆ haloalkyl, and C₁-C₆ haloalkoxy), C₃-C₇ carbocyclylthio (optionally substituted with halo, C₁-C₆ alkyl, C₁-C₆ alkoxy, C₁-C₆ haloalkyl, and C₁-C₆ haloalkoxy), 3-10 membered heterocyclyl-thio (optionally substituted with halo, C₁-C₆ alkyl, C₁-C₆ alkoxy, C₁-C₆ haloalkyl, and C₁-C₆ haloalkoxy), 5-10 membered heteroaryl-thio (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkylthio (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkylthio (optionally substituted with halo, C₁-C₆ alkyl, C₁-C₆ alkoxy, C₁-C₆ haloalkyl, and C₁-C₆ haloalkoxy), aryl(C₁-C₆)alkylthio (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkylthio (optionally substituted with halo, C₁-C₆ alkyl, C₁-C₆ alkoxy, C₁-C₆ haloalkyl, and C₁-C₆ haloalkoxy), amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

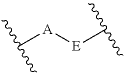

includes the substituent being oriented such that the A is attached at the leftmost attachment point of the molecule as well as the case in which A is attached at the rightmost attachment point of the molecule.

A “sweetener”, “sweet flavoring agent”, “sweet flavor entity”, or “sweet compound” herein refers to a compound or ingestibly acceptable salt thereof that elicits a detectable sweet flavor in a subject, e.g., a compound that activates a T1R2/T1R3 receptor in vitro.

### Sweeteners

Sweeteners have a wide range of chemically distinct structures and hence possess varying properties, such as, without limitation, odor, flavor, mouthfeel, and aftertaste. Compositions described herein include any one or more sweetener, including combinations of any one or more sweetener disclosed here.

Natural or artificial sweeteners for use in the ingestible composition comprising a sweetener in combination with a flavor enhancer include but are not limited to natural or synthetic carbohydrates or carbohydrate analogues, including monosaccharides, disaccharides, oligosaccharides, and polysaccharides, and including rare sugars, or sugars in either of the D- or L-conformations, and include, for example, sucrose, fructose, glucose, L-arabinose, L-fucose, L-glucose, L-ribose, D-arabino-hexulose, psicose, altrose, arabinose, turanose, abequose, allose, abrusoside A, aldotriose, threose, xylose, xylulose, xylo-oligosaccharide (such as xylotriose and xylobiose), lyxose, polydextrose, oligofructose, fucose, galacto-oligosaccharide, galactosamine, galactose, gentio-oligosaccharide (such as gentiobiose, gentiotriose, and gentiotetraose), dextrose, cellobiose, D-leucrose, D-psicose, D-ribose, D-tagatose, trehalose (mycose), neotrehalose, isotrehalose, raffinose, idose, tagatose, melibiose, mannan-oligosaccharide, rhamnose, ribose, ribulose, malto-oligosaccharide (such as maltotriose, maltotetraose, maltopentaose, maltohexaose, and maltoheptaose), maltose, sucrose acetate isobutyrate, dextrose, erythrose, erythrulose, deoxyribose, gulose, ketotriose, lactose, lactulose, kestose, nystose, mannose, sucralose, palatinose, polydextrose, sorbose, sugaridextrose (blended sugar), or talose, or combinations of any two or more of the aforementioned sweeteners.

The one or more sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The one or more sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, Stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside 0, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In still other embodiments, the one or more sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners.

One of skill in the art will recognize that any one or more of any of the aforementioned sweeteners can be combined in various ratios, amounts, or concentrations to yield a sweetener alone or a combination of two or more sweeteners, which is then combined with one or more flavor modifying compound.

One of skill in the art will recognize that the aforementioned sweeteners for use in a formulation comprising one or more sweetener and one or more flavor modifying compound are provided by way of example and are not intended to be limiting.

### Flavor Modifying Compound

In some embodiments, additional flavor modifying compounds may be combined with the compounds described herein. In some embodiments, the one or more flavor modifying compound is one or more flavor modifying compound as disclosed in U.S. Application Publication No. 2005/0084506, entitled “Novel Flavors, Flavor Modifiers, Tastants, Taste Enhancers, Umami or Sweet Tastants, and/or Enhancers and Use Thereof”, filed Aug. 6, 2004, which is incorporated herein by reference in its entirety. In some embodiments, the one or more flavor modifying compound is one or more flavor modifying compound as disclosed in U.S. Application Publication No. 2007/0003680, entitled “Bis-Aromatic Amides and Their Uses as Sweet Flavor Modifiers, Tastants, and Taste Enhancers”, filed Jun. 15, 2006, which is incorporated herein by reference in its entirety. In some embodiments, the one or more flavor modifying compound is one or more flavor modifying compound as disclosed in U.S. Application Publication No. 2008/0306093, entitled “Modulation of Chemosensory Receptors and Ligands Associated Therewith”, filed Jun. 8, 2007, which is incorporated herein by reference in its entirety. In some embodiments, the one or more flavor modifying compound is one or more flavor modifying compound as disclosed in U.S. Application Publication No. 2011/0224155, entitled “Modulation of Chemosensory Receptors and Ligands Associated Therewith”, filed Apr. 14, 2011, which is incorporated herein by reference in its entirety. In some embodiments, the one or more flavor modifying compound is one or more flavor modifying compound as disclosed in U.S. Application Publication No. 2011/0245353, entitled “Sweet Flavor Modifier”, filed Mar. 31, 2011, which is incorporated herein by reference in its entirety. In some embodiments, the one or more flavor modifying compound is one or more flavor modifying compound as disclosed in U.S. Application Publication No. 2013/0041046, entitled “Sweet Flavor Modifier”, filed Aug. 10, 2012, which is incorporated herein by reference in its entirety. In some embodiments, the one or more flavor modifying compound is one or more flavor modifying compound as disclosed in U.S. Application Publication No. 2014/0094453, entitled “Sweet flavor modifier”, filed Dec. 4, 2014, which is incorporated herein by reference in its entirety. In some embodiments, the one or more flavor modifying compound is one or more flavor modifying compound as disclosed in U.S. Application Publication No. 2014/0235624, entitled “Sweet flavor modifier”, filed Feb. 19, 2014, which is incorporated herein by reference in its entirety. In some embodiments, the one or more flavor modifying compound is one or more flavor modifying compound as disclosed in U.S. Application Publication No. 2015/0376176, entitled “Sweet flavor modifier”, filed Aug. 14, 2015, which is incorporated herein by reference in its entirety. In some embodiments, the one or more flavor modifying compound is one or more flavor modifying compound as disclosed in U.S. Application Publication No. 2016/0185727, entitled “Substituted 4-amino-5-(cyclohexyloxy)quinolone-3-carboxylic acids as Sweet Flavor Modifiers”, filed Oct. 28, 2015, which is incorporated herein by reference in its entirety.

In some embodiments, the one or more flavor modifying compound is a compound having a cyclic thiadiazine core.

Some embodiments include a combination of

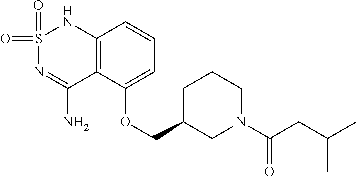 [SMILES: CC(C)CC(=O)N1CCC[C@H](COc2cccc3c2C(N)=NS(=O)(=O)N3)C1]

together with any one or more of the flavor modifying compounds described or referenced herein.

Some embodiments include a combination of

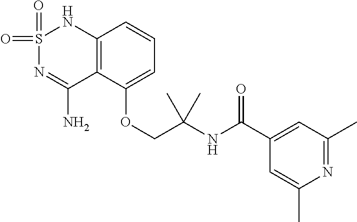 [SMILES: Cc1cc(C(=O)NC(C)(C)COc2cccc3c2C(N)=NS(=O)(=O)N3)cc(C)n1]

together with any one or more of the flavor modifying compounds described or referenced herein.

Some embodiments include a combination

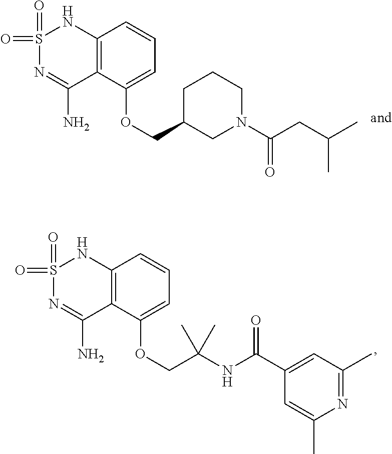 [SMILES: CC(C)CC(=O)N1CCC[C@H](COc2cccc3c2C(N)=NS(=O)(=O)N3)C1 ; Cc1cc(C(=O)NC(C)(C)COc2cccc3c2C(N)=NS(=O)(=O)N3)cc(C)n1]

together with any one or more additional flavor modifying compound described or referenced herein.

Some embodiments include a combination of

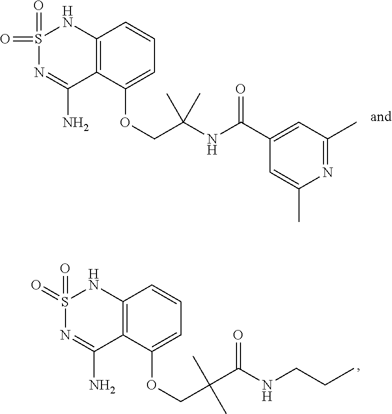 [SMILES: Cc1cc(C(=O)NC(C)(C)COc2cccc3c2C(N)=NS(=O)(=O)N3)cc(C)n1 ; CCCNC(=O)C(C)(C)COc1cccc2c1C(N)=NS(=O)(=O)N2]

together with any one or more additional flavor modifying compound described or referenced herein.

Some embodiments include a combination of

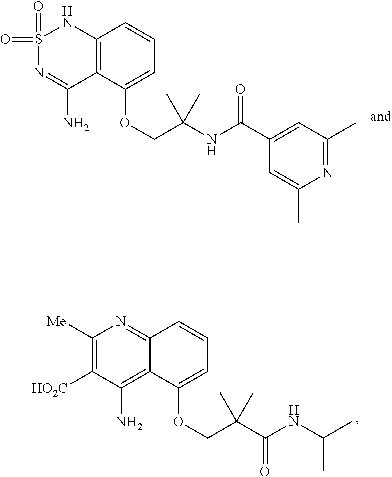 [SMILES: Cc1cc(C(=O)NC(C)(C)COc2cccc3c2C(N)=NS(=O)(=O)N3)cc(C)n1 ; Cc1nc2cccc(OCC(C)(C)C(=O)NC(C)C)c2c(N)c1C(=O)O]

together with any one or more additional flavor modifying compound described or referenced herein.

Some embodiments include any combination of flavor modifying compounds as described herein together with any one or more sweeteners as described herein.

In some embodiments, the one or more flavor modifying compound is present at an amount from about 0.001 ppm to 500 ppm. Thus, in some alternatives, the amount of the one or more flavor modifying compound is 0.001, 0.01, 0.1, 1, 5, 10, 20, 50, 100, 200, 300, 400, or 500 ppm or a value that is within a range defined by any two of the aforementioned values.

### Compositions and Uses

Some compositions herein relate to formulations comprising one or more sweetener as disclosed herein in combination with one or more flavor modifying compounds as described herein. Thus, it is recognized that the combinations of one or more sweeteners with one or more flavor modifying compounds as disclosed herein are non-limiting. In some embodiments, said formulations comprising one or more sweetener and one or more flavor modifying compounds are used as compositions for ingestible or non-ingestible products.

A formulation comprising one or more sweetener in combination with one or more flavor modifying compound can be used for modulating a chemosensory receptor and/or its ligand, including modulating the activity, structure, function, expression, and/or modification of a chemosensory receptor as well as modulating, treating, or taking prophylactic measure of a condition, e.g., physiological or pathological condition, associated with a chemosensory receptor. In general, a physiological or pathological condition associated with a chemosensory receptor includes a condition, disease, or disorder associated with the chemosensory receptor and/or its ligand, e.g., gastrointestinal disorders, metabolic disorders, functional gastrointestinal disorders, etc. In some embodiments, the formulation increases or enhances sweet flavor. In another embodiment, the formulation modulates a sweet receptor and/or its ligand expressed in a place of the body other than the taste buds, such as an internal organ. In general, the formulations of the present disclosure can be provided in a composition, such as, e.g., an ingestible composition. In some embodiments, the present formulation can impart a more sugar-like temporal profile and/or flavor profile to a sweetener composition by combining one or more flavor modifying compound with one or more sweetener in the combined formulation. In another embodiment, the present formulation can increase or enhance the sweet taste of a composition by contacting the composition thereof with one or more present formulation to form a modified composition. In another embodiment, the present formulations can be in a composition that modulates the sweet receptors and/or their ligands expressed in the body other than in the taste buds.

In some embodiments, the present formulations have sucrose modifying behavior and/or sweet agonist behavior in vitro and/or in vivo (e.g., as shown in sensory studies). In some embodiments, the present formulations demonstrate a favorable side-taste profile in vivo.

Whether or not a formulation exhibits sweet modifying/agonist effects can be determined by any suitable test method. For example, the formulation comprising one or more sweeteners in combination with one or more flavor modifying compound can be evaluated in a sensory test using a human taste panel.

In some embodiments, the formulation may be diluted before being tested. In some embodiments, the formulation is diluted for about 2 times, about 5, about 10, about 50, about 100, about 200, about 300, about 400, about 500, about 1000, or more times before being tested.

The tests can be conducted with and/or without additives. In some embodiments, the formulation is tested to evaluate the sweet enhancement to one or more additives. In the test, the participants can provide their impression as to the similarities of the characteristics of the sweetener compositions, with and/or without additives, with those comprising sugar. A suitable procedure for determining whether a composition has a more sugar-like taste is through the use of a panel of assessors, who measure the sweetness of the formulations.

One embodiment provides a formulation for use in a method of preparing a ready-to-use composition, such as a final food or beverage product, or animal feed product. The method comprises contacting a first composition, such as a first food or beverage product that may contain one or more sweetener described herein, with a flavoring concentrate composition or formulation (e.g., solid or liquid) comprising one or more flavor modifying compound to form the ready-to-use composition.

In some embodiments, an ingestible composition may be a beverage. In some embodiments, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, chocolate milk, dairy only flavored milk drinks, flavored milk drinks with fruit juice, milk alternative beverages including rice milk, almond milk, cashew milk, coconut milk, hazelnut milk, hemp milk, pistachio milk, oat milk, wheat milk, barley milk, millet milk, spelt milk, triticale milk, and soy milk, sour milk drinks, fermented dairy drinks, kefir, beverages containing cereal extracts and smoothies. In some embodiments, the beverage may be a soft drink.

In some embodiments, one or more flavor modifying compound as described herein and one or more sweetener as described herein may be included in a food or beverage product, wherein the food or beverage product may additionally comprise:
acids, including, for example citric acid, phosphoric acid, ascorbic acid, sodium acid sulfate, lactic acid, or tartaric acid;
bitter ingredients, including, for example caffeine, quinine, green tea, catechins, polyphenols, green robusta coffee extract, green coffee extract, whey protein isolate, or potassium chloride;
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, Panax ginseng extract, guanana extract, ginger extract, L-phenylalanine, L-carnitine, L-tartrate, D-glucoronolactone, inositol, bioflavonoids, Echinacea, Ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; and
stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), or carrageenan.

Some embodiments provide supplements, nutraceuticals, functional food products (e.g., any fresh or processed food claimed to have a health-promoting and/or disease-preventing properties beyond the basic nutritional function of supplying nutrients), animal feed products, pharmaceutical product, over the counter (OTC) product, oral care product, cosmetic products such as sweetened lip balms, and other personal care products including one or more flavor modifying compound as described herein and sweetener as described herein.

In general, over the counter (OTC) product and oral care product generally refer to product for household and/or personal use which may be sold without a prescription and/or without a visit to a medical professional. Examples of the OTC products include, but are not limited to Vitamins and dietary supplements; Topical analgesics and/or anesthetic; Cough, cold and allergy remedies; Antihistamines and/or allergy remedies; and combinations thereof. Vitamins and dietary supplements include, but are not limited to vitamins, dietary supplements, tonics/bottled nutritive drinks, child-specific vitamins, dietary supplements, any other products of or relating to or providing nutrition, and combinations thereof. Topical analgesics and/or anesthetic include any topical creams/ointments/gels used to alleviate superficial or deep-seated aches and pains, e.g., muscle pain; teething gel; patches with analgesic ingredient; and combinations thereof. Cough, cold and allergy remedies include, but are not limited to decongestants, cough remedies, pharyngeal preparations, medicated confectionery, antihistamines and child-specific cough, cold and allergy remedies; and combination products. Antihistamines and/or allergy remedies include, but are not limited to any systemic treatments for hay fever, nasal allergies, insect bites and stings. Examples of oral care product include, but are not limited to mouth cleaning strips, toothpaste, toothbrushes, mouthwashes/dental rinses, denture care, mouth fresheners at-home teeth whiteners, dentifrices, and dental floss.

In some embodiments, a one or more flavor modifying compound as described herein and one or more sweetener as described herein may be included in food or beverage products or formulations. Examples of food and beverage products or formulations include, but are not limited to sweet coatings, frostings, or glazes for ingestible products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non-alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, flavored alcoholic beverages, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water, purified/table water, and vitamin water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; gelato; sorbet; sherbet; soy, oat, bean (e.g., red bean and mung bean), coconut, nut and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, speculoos spreads, butters, flavored butters, margarine, edible oil spreads, oleos, cheese or cream cheese spreads, savory spread, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for ingestible compositions, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, chocolate milk, dairy only flavored milk drinks, flavored milk drinks with fruit juice, milk alternative beverages including rice milk, almond milk, cashew milk, coconut milk, hazelnut milk, hemp milk, pistachio milk, oat milk, wheat milk, barley milk, millet milk, spelt milk, triticale milk, and soy milk, sour milk drinks, fermented dairy drinks, kefir, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, speculoos spreads, butters, flavored butters, margarine, edible oil spreads, oleos, cheese or cream cheese spreads, savory spread, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including one or more flavor modifying compound as described herein and sweetener as described herein.

In some embodiments, a one or more flavor modifying compound as described herein and one or more sweetener as described herein may be provided in a flavoring concentrate formulation, e.g., suitable for subsequent processing to produce a ready-to-use (for example, ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

In one embodiment, the flavoring concentrate formulation comprises i) one or more flavor modifying compound as described herein; ii) a carrier; and iii) optionally at least one adjuvant. The term “carrier” denotes a usually inactive accessory substance, such as solvents, binders, or other inert medium, which is used in combination with the one or more flavor modifying compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present disclosure. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described above. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation includes the one or more flavor modifying compound in a concentration that is at least 2 times of the concentration of the compound in a ready-to-use composition. In one embodiment, the flavoring concentrate formulation includes the one or more flavor modifying compound in a concentration that is at least 5 times of the concentration of the compound in a ready-to-use composition. In one embodiment, the flavoring concentrate formulation includes the one or more flavor modifying compound in a concentration that is at least 10 times of the concentration of the compound in a ready-to-use composition. In one embodiment, the flavoring concentrate formulation includes the one or more flavor modifying compound in a concentration that is at least 15 times of the concentration of the compound in a ready-to-use composition. In one embodiment, the flavoring concentrate formulation includes the one or more flavor modifying compound in a concentration that is at least 20 times of the concentration of the compound in a ready-to-use composition. In one embodiment, the flavoring concentrate formulation includes the one or more flavor modifying compound in a concentration that is at least 30 times of the concentration of the compound in a ready-to-use composition. In one embodiment, the flavoring concentrate formulation includes the one or more flavor modifying compound in a concentration that is at least 40 times of the concentration of the compound in a ready-to-use composition. In one embodiment, the flavoring concentrate formulation includes the one or more flavor modifying compound in a concentration that is at least 50 times of the concentration of the compound in a ready-to-use composition. In one embodiment, the flavoring concentrate formulation includes the one or more flavor modifying compound in a concentration that is at least 60 times of the concentration of the compound in a ready-to-use composition. In one embodiment, the flavoring concentrate formulation includes the one or more flavor modifying compound in a concentration that is up to 100 times of the concentration of the compound in a ready-to-use composition.

### Ingestible Compositions

In some embodiments, compounds as disclosed and described herein, individually or in combination, can be used for one or more methods such as modifying receptor function associated with chemosensory or chemosensory related sensation or reaction. Some embodiments provide a method of modulating a chemosensory receptor includes modulating the activity, structure, function, and/or modification of a chemosensory receptor as well as modulating, treating, or taking prophylactic measure of a condition, e.g., physiological or pathological condition, associated with a chemosensory receptor. In general, a physiological or pathological condition associated with a chemosensory receptor includes a condition, disease, or disorder associated with the chemosensory receptor and/or its ligand, e.g.; gastrointestinal disorders, metabolic disorders, functional gastrointestinal disorders, etc. In one embodiment, the method includes increasing or enhancing sweet flavor. In another embodiment, the method includes modulating a sweet receptor and/or its ligand expressed in a place of the body other than the taste buds, such as an internal organ.

In general, compounds as disclosed and described herein, individually or in combination, can be provided in a composition, such as, e.g., an ingestible composition. In one embodiment, compounds as disclosed and described herein, individually or in combination, can impart a more sugar-like temporal profile and/or flavor profile to a sweetener composition by combining one or more of the compounds as disclosed and described herein with one or more sweeteners in the sweetener composition. In another embodiment, compounds as disclosed and described herein, individually or in combination, can increase or enhance the sweet taste of a composition by contacting the composition thereof with the compounds as disclosed and described herein to form a modified composition. In another embodiment, compounds as disclosed and described herein, individually or in combination, can be in a composition that modulates the sweet receptors and/or their ligands expressed in the body other than in the taste buds.

Some embodiments provide an ingestible composition, comprising the compound of any one of formulas (I), (Ia), (Ib), (Ic), (Id), (Je), and (If), and a sweetener. In some embodiments, the composition further comprises a vehicle. In some embodiments, the vehicle is water. In some embodiments, the compound may be present at a concentration at or below its sweetness recognition threshold. In some embodiments, the sweetener is present in an amount from about 0.1% to about 12% by weight. In some embodiments, the sweetener is present in an amount from about 0.2% to about 10% by weight. In some embodiments, the sweetener is present in an amount from about 0.3% to about 8% by weight. In some embodiments, the sweetener is present in an amount from about 0.4% to about 6% by weight. In some embodiments, the sweetener is present in an amount from about 0.5% to about 5% by weight. In some embodiments, the sweetener is present in an amount from about 1% to about 2% by weight. In some embodiments, the sweetener is present in an amount from about 0.1% to about 5% by weight. In some embodiments, the sweetener is present in an amount from about 0.1% to about 4% by weight. In some embodiments, the sweetener is present in an amount from about 0.1% to about 3% by weight. In some embodiments, the sweetener is present in an amount from about 0.1% to about 2% by weight. In some embodiments, the sweetener is present in an amount from about 0.1% to about 1% by weight. In some embodiments, the sweetener is present in an amount from about 0.1% to about 0.5% by weight. In some embodiments, the sweetener is present in an amount from about 0.5% to about 10% by weight. In some embodiments, the sweetener is present in an amount from about 2% to about 8% by weight. In some embodiments, the sweetener may be common saccharide sweeteners, e.g., sucrose, fructose, glucose, and sweetener compositions comprising natural sugars, such as corn syrup (including high fructose corn syrup) or other syrups or sweetener concentrates derived from natural fruit and vegetable sources; rare natural sugars including D-allose, D-psicose, L-ribose, D-tagatose, L-glucose, L-fucose, L-arbinose, D-turanose, and D-leucrose; semi-synthetic “sugar alcohol” sweeteners such as erythritol, isomalt, lactitol, mannitol, sorbitol, xylitol, maltodextrin, and the like; and artificial sweeteners such as aspartame, saccharin, acesulfame-K, cyclamate, sucralose, and alitame. In some embodiments, the sweetener may be selected from the group consisting of cyclamic acid, mogroside, tagatose, maltose, galactose, mannose, sucrose, fructose, lactose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A and other sweet Stevia-based glycosides, carrelame and other guanidine-based sweeteners. In some embodiments, the sweetener may combinations of two or more sweeteners as disclosed herein. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose. In some embodiments, the sugar may be a combination of about 55% fructose and about 42% glucose. In some embodiments, the sugar may be a combination of about 42% fructose and about 53% glucose. In some embodiments, the sugar may be a combination of about 90% fructose and about 10% glucose. In some embodiments, the sweetener may be a rare sugar. In some embodiments, the rare sugar is selected from the group consisting of D-allose, D-psicose, L-ribose, D-tagatose, L-glucose, L-fucose, L-arbinose, D-turanose, D-leucrose and combinations thereof. In some embodiments, the rare sugar is D-psicose. In some embodiments, the rare sugar is D-tagatose. In some embodiments, the sweetener is an artificial sweetener. In some embodiments, the artificial sweetener may be sucralose.

In some embodiments, an ingestible composition may be a beverage. In some embodiments, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In one embodiment, compounds as disclosed and described herein, individually or in combination, can be used at its ligand enhancing concentrations, e.g., very low concentrations on the order of a few parts per million, in combination with one or more known sweeteners, natural or artificial, so as to reduce the concentration of the known sweetener required to prepare an ingestible composition having the desired degree of sweetness.

In one embodiment, compounds as disclosed and described herein, individually or in combination, can enhance the sweetness of a sweetener under a broad range of pH, e.g., from lower pH to neutral pH. The lower and neutral pH includes, but is not limited to, a pH from about 2.5 to about 8.5; from about 3.0 to about 8.0; from about 3.5 to about 7.5; and from about 4.0 to about 7. In certain embodiments, compounds as disclosed and described herein, individually or in combination, can enhance the perceived sweetness of a fixed concentration of a sweetener in taste tests at a compound concentration of about 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination. In some embodiments, the sweetener may be common saccharide sweeteners, e.g., sucrose, fructose, glucose, and sweetener compositions comprising natural sugars, such as corn syrup (including high fructose corn syrup) or other syrups or sweetener concentrates derived from natural fruit and vegetable sources; rare natural sugars including D-allose, D-psicose, L-ribose, D-tagatose, L-glucose, L-fucose, L-arbinose, D-turanose, and D-leucrose; semi-synthetic “sugar alcohol” sweeteners such as erythritol, isomalt, lactitol, mannitol, sorbitol, xylitol, maltodextrin, and the like; and artificial sweeteners such as aspartame, saccharin, acesulfame-K, cyclamate, sucralose, and alitame. In some embodiments, the sweetener may be selected from the group consisting of cyclamic acid, mogroside, tagatose, maltose, galactose, mannose, sucrose, fructose, lactose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A and other sweet Stevia-based glycosides, carrelame and other guanidine-based sweeteners. In some embodiments, the sweetener may combinations of two or more sweeteners as disclosed herein. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener may be a sugar. In some embodiments, the sugar is cane sugar. In some embodiments, the sugar is beet sugar. In some embodiments, the sweetener may be a combination of one or more sugars and other natural and artificial sweeteners. In some embodiments, the sugar may be sucrose, fructose, glucose or combinations thereof (for example, high fructose corn syrup). In some embodiments, the sugar may be sucrose. In some embodiments, the sugar may be a combination of fructose and glucose. In some embodiments, the sugar may be a combination of about 55% fructose and about 42% glucose. In some embodiments, the sugar may be a combination of about 42% fructose and about 53% glucose. In some embodiments, the sugar may be a combination of about 90% fructose and about 10% glucose. In some embodiments, the sweetener may be a rare sugar. In some embodiments, the rare sugar is selected from the group consisting of D-allose, D-psicose, L-ribose, D-tagatose, L-glucose, L-fucose, L-arbinose, D-turanose, D-leucrose and combinations thereof. In some embodiments, the rare sugar is D-psicose. In some embodiments, the rare sugar is D-tagatose. In some embodiments, the sweetener is an artificial sweetener. In some embodiments, the artificial sweetener is sucralose.

Typically at least a sweet receptor modulating amount, a sweet receptor ligand modulating amount, a sweet flavor modulating amount, a sweet flavoring agent amount, a sweet flavor enhancing amount, or a therapeutically effective amount of one or more of the present compounds will be added to the ingestible composition, optionally in the presence of sweeteners so that the sweet flavor modified ingestible composition has an increased sweet taste as compared to the ingestible composition prepared without the compounds of the present invention, as judged by human beings or animals in general, or in the case of formulations testing, as judged by a majority of a panel of at least eight human taste testers, via procedures commonly known in the field.

In some embodiments, compounds as disclosed and described herein, individually or in combination, modulate the sweet taste or other taste properties of other natural or synthetic sweet tastants, and ingestible compositions made therefrom. In one embodiment, the compounds as disclosed and described herein, individually or in combination, may be used or provided in its ligand enhancing concentration(s). For example, the compounds as disclosed and described herein, individually or in combination, may be present in an amount of from about 0.001 ppm to 100 ppm, or narrower alternative ranges from about 0.1 ppm to about 10 ppm, from about 0.01 ppm to about 30 ppm, from about 0.05 ppm to about 10 ppm, from about 0.01 ppm to about 5 ppm, or from about 0.02 ppm to about 2 ppm, or from about 0.01 ppm to about 1 ppm.

Some embodiments provide a sweet enhancing composition. The sweet enhancing composition comprises a compound of the present invention in a sweet flavor enhancing amount in combination with a first amount of sweetener, wherein the sweetening is more than the sweetening provided by the first amount of sweetener without the compound. In some embodiments, the sweetener may be common saccharide sweeteners, e.g., sucrose, fructose, glucose, and sweetener compositions comprising natural sugars, such as corn syrup (including high fructose corn syrup) or other syrups or sweetener concentrates derived from natural fruit and vegetable sources; rare natural sugars including D-allose, D-psicose, L-ribose, D-tagatose, L-glucose, L-fucose, L-arbinose, D-turanose, and D-leucrose; semi-synthetic “sugar alcohol” sweeteners such as erythritol, isomalt, lactitol, mannitol, sorbitol, xylitol, maltodextrin, and the like; and artificial sweeteners such as aspartame, saccharin, acesulfame-K, cyclamate, sucralose, and alitame. In some embodiments, the sweetener may be selected from the group consisting of cyclamic acid, mogroside, tagatose, maltose, galactose, mannose, sucrose, fructose, lactose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A and other sweet Stevia-based glycosides, carrelame and other guanidine-based sweeteners. In some embodiments, the sweetener may combinations of two or more sweeteners as disclosed herein. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener may be a sugar. In some embodiments, the sugar is cane sugar. In some embodiments, the sugar is beet sugar. In some embodiments, the sweetener may be a combination of one or more sugars and other natural and artificial sweeteners. In some embodiments, the sugar may be sucrose, fructose, glucose or combinations thereof (for example, high fructose corn syrup). In some embodiments, the sugar may be sucrose. In some embodiments, the sugar may be a combination of fructose and glucose. In some embodiments, the sugar may be a combination of about 55% fructose and about 42% glucose. In some embodiments, the sugar may be a combination of about 42% fructose and about 53% glucose. In some embodiments, the sugar may be a combination of about 90% fructose and about 10% glucose. In some embodiments, the sweetener may be a rare sugar. In some embodiments, the rare sugar is selected from the group consisting of D-allose, D-psicose, L-ribose, D-tagatose, L-glucose, L-fucose, L-arbinose, D-turanose, D-leucrose and combinations thereof. In some embodiments, the rare sugar is D-psicose. In some embodiments, the rare sugar is D-tagatose. In some embodiments, the sweetener is an artificial sweetener. In some embodiments, the artificial sweetener may be sucralose.

In some embodiments, compounds as disclosed and described herein, individually or in combination, provide enhancement of potency of a sweetener at the T1R2/T1R3 taste receptor as measured by an enhancement ratio, defined as the ratio of EC₅₀ of the sweetener with and without the compound described herein. In some embodiments, compounds as disclosed and described herein, individually or in combination, provide enhancement ratio of greater than 1 and less than 10. In some embodiments, compounds as disclosed and described herein, individually or in combination, provide an enhancement ratio from 10 to 20. In some embodiments, compounds as disclosed and described herein, individually or in combination, provide an enhancement ratio greater than 20. In some embodiments, the sweetener may be common saccharide sweeteners, e.g., sucrose, fructose, glucose, and sweetener compositions comprising natural sugars, such as corn syrup (including high fructose corn syrup) or other syrups or sweetener concentrates derived from natural fruit and vegetable sources; rare natural sugars including D-allose, D-psicose, L-ribose, D-tagatose, L-glucose, L-fucose, L-arbinose, D-turanose, and D-leucrose; semi-synthetic “sugar alcohol” sweeteners such as erythritol, isomalt, lactitol, mannitol, sorbitol, xylitol, maltodextrin, and the like; and artificial sweeteners such as aspartame, saccharin, acesulfame-K, cyclamate, sucralose, and alitame. In some embodiments, the sweetener may be selected from the group consisting of cyclamic acid, mogroside, tagatose, maltose, galactose, mannose, sucrose, fructose, lactose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A and other sweet Stevia-based glycosides, carrelame and other guanidine-based sweeteners. In some embodiments, the sweetener may combinations of two or more sweeteners as disclosed herein. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener may be a sugar. In some embodiments, the sweetener may be a combination of one or more sugars and other natural and artificial sweeteners. In some embodiments, the sugar may be sucrose, fructose, glucose or combinations thereof (for example, high fructose corn syrup). In some embodiments, the sugar may be sucrose. In some embodiments, the sugar may be a combination of fructose and glucose. In some embodiments, the sugar may be a combination of about 55% fructose and about 42% glucose. In some embodiments, the sugar may be a combination of about 42% fructose and about 53% glucose. In some embodiments, the sugar may be a combination of about 90% fructose and about 10% glucose. In some embodiments, the sweetener may be a rare sugar. In some embodiments, the rare sugar is selected from the group consisting of D-allose, D-psicose, L-ribose, D-tagatose, L-glucose, L-fucose, L-arbinose, D-turanose, D-leucrose and combinations thereof. In some embodiments, the rare sugar is D-psicose. In some embodiments, the rare sugar is D-tagatose. In some embodiments, the sweetener is an artificial sweetener. In some embodiments, the artificial sweetener may be sucralose.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

### Therapeutic Utilities

In some embodiments, compounds as disclosed and described herein, individually or in combination can be used for therapeutic purpose such as modulating a chemosensory receptor and/or its ligand to achieve therapeutic effect. For example, the therapeutic purpose may include modulating a chemosensory receptor and/or its ligand expressed in the body other than in the taste buds.

In some embodiments, a method of modulating a chemosensory receptor and/or its ligand includes modulating the expression, secretion, and/or functional level of T1R expressing cells associated with hormone, peptide, enzyme production by administering compounds as disclosed and described herein, individually or in combination to an individual in need thereof. In one example, the method of the present invention includes modulating the level of glucose, e.g., inhibitors or modulators of a chemosensory receptor such as T1R2/T1R3 can be used to decrease glucose level (e.g., glucose absorption) in a subject by administering compounds as disclosed and described herein, individually or in combination to an individual in need thereof. In some embodiments, the method includes modulating the level of incretins, e.g., agonists or enhancers of a chemosensory receptor such as T1R2/T1R3 can be used to increase glucagon-like peptide 1 (GLP-1) and thus increase the production of insulin by administering compounds as disclosed and described herein, individually or in combination to an individual in need thereof. In some embodiments, the method includes modulating the expression, secretion, and/or activity level of hormones or peptides produced by T1R expressing cells or gastrointestinal hormone producing cells, e.g., ligands for 5HT receptors (e.g., serotonin), incretins (e.g., GLP-1 and glucose-dependent insulinotropic polypeptide (GIP)), gastrin, secretin, pepsin, cholecystokinin, amylase, ghrelin, leptin, somatostatin, etc. by administering compounds as disclosed and described herein, individually or in combination to an individual in need thereof. In some embodiments, the method includes modulating the pathways associated with hormones, peptides, and/or enzymes secreted by TiR expressing cells by administering compounds as disclosed and described herein, individually or in combination to an individual in need thereof.

In some embodiments, the method includes modulating the activity of T1R (e.g., T1R1, T1R2, or T1R3) expressing cells, e.g., liver cells (e.g., hepatocytes, endothelial cells, Kupffer cells, Stellate cells, epithelial cells of bile duct, etc.), heart cells (e.g., endothelial, cardiac, and smooth muscle cells, etc.), pancreatic cells (e.g., alpha cell, beta cell, delta cell, neurosecretory PP cell, D1 cell, etc.), cells in the nipple (e.g., ductal epithelial cells, etc.), stomach cells (e.g., mucous cells, parietal cells, chief cells, G cells, P/D1 cells), intestinal cells (e.g., enteroendocrine cells, brush cells, etc.), salivary gland cells (e.g., Seromucous cells, mucous cells, myoepithelial cells, intercalated duct cell, striated duct cell, etc.), L cells (e.g., expressing GLP-1, etc.), enterochromaffin cells (e.g., expressing serotonin), enterochromaffin-like cells, G cells (e.g., expressing gastrin), D cells (delta cells, e.g., expressing somatostatin), I cells (e.g., expressing cholescystokinin (CCK), K cells (e.g., expressing gastric inhibitory polypeptide), P/D1 cells (e.g., expressing ghrelin), chief cells (e.g., expressing pepsin), and S cells (e.g., expressing secretin) by administering compounds as disclosed and described herein, individually or in combination to an individual in need thereof. In some embodiments, the method includes increasing the expression level of T1R in T1R expressing cells by administering compounds as disclosed and described herein, individually or in combination to an individual in need thereof. In some embodiments, the method includes increasing the secretion level of T1R expressing cells by administering compounds as disclosed and described herein, individually or in combination to an individual in need thereof.

In some embodiments, the method includes modulation, treatment, and/or prophylactic measure of a condition associated with the gastrointestinal system including without any limitation conditions associated with esophageal motility (e.g., cricopharyngeal achalasia, globus hystericus, achalasia, diffuse esophageal spasm and related motor disorders, scleroderma involving the esophagus, etc.), inflammatory disorders (e.g., gastroesophageal reflux and esophagitis, infectious esophagitis, etc.), peptic ulcer, duodenal ulcer, gastric ulcer, gastrinoma, stress ulcers and erosions, drug-associated ulcers and erosions, gastritis, esophageal cancer, tumors of the stomach, disorders of absorption (e.g., absorption of specific nutrients such as carbohydrate, protein, amino acid, fat, cholesterol and fat-soluble vitamins, water and sodium, calcium, iron, water-soluble vitamins, etc.), disorders of malabsorption, defects in mucosal function (e.g., inflammatory or infiltrative disorders, biochemical or genetic abnormalities, endocrine and metabolic disorders, protein-losing enteropathy, etc.), autoimmune diseases of the digestive tract (e.g., celiac disease, Crohn's disease, ulcerative colitis, etc.), irritable bowel syndrome, inflammatory bowel disease, complications of inflammatory bowel disease, extraintestinal manifestations of inflammatory bowel disease, disorders of intestinal motility, vascular disorders of the intestine, anorectial disorders (e.g., hemorrhoids, anal inflammation, etc.), colorectal cancer, tumors of the small intestine, cancers of the anus, derangements of hepatic metabolism, hyperbilirubinemia, hepatitis, alcoholic liver disease and cirrhosis, biliary cirrhosis, neoplasms of the liver, infiltrative and metabolic diseases affecting the liver (e.g., fatty liver, reye's syndrome, diabetic glycogenosis, glycogen storage disease, Wilson's disease, hemochromatosis), diseases of the gallbladder and bile ducts, disorders of the pancreas (e.g., pancreatitis, pancreatic exocrine insufficiency, pancreatic cancer, etc.), endocrine tumors of the gastrointestinal tract and pancreas, etc. by administering compounds as disclosed and described herein, individually or in combination to an individual in need thereof.

In some embodiments, the method includes modulation, treatment, and/or prophylactic measure of a condition associated with metabolic disorders, e.g., appetite, body weight, food or liquid intake or a subject's reaction to food or liquid intake, or state of satiety or a subject's perception of a state of satiety, nutrition intake and regulation, (e.g., protein-energy malnutrition, physiologic impairments associated with protein-energy malnutrition, etc.), obesity, secondary obesity (e.g., hypothyroidism, Cushing's disease, insulinoma, hypothalamic disorders, etc.), eating disorders (e.g., anorexia nervosa, bulimia, etc.), vitamin deficiency and excess, insulin metabolism, diabetes (type I and type II) and complications thereof (e.g., circulatory abnormalities, retinopathy, diabetic nephropathy, diabetic neuropathy, diabetic foot ulcers, etc.), glucose metabolism, fat metabolism, hypoglycemia, hyperglycemia, hyperlipoproteinemias, etc. by administering compounds as disclosed and described herein, individually or in combination to an individual in need thereof

In some embodiments, the method includes modulation, treatment, and/or prophylactic measure of a condition associated with functional gastrointestinal disorders, e.g., in the absence of any particular pathological condition such as peptic ulcer and cancer, a subject has abdominal dyspepsia, e.g., feeling of abdominal distention, nausea, vomiting, abdominal pain, anorexia, reflux of gastric acid, or abnormal bowel movement (constipation, diarrhea and the like), optionally based on the retention of contents in gastrointestinal tract, especially in stomach. In one example, functional gastrointestinal disorders include a condition without any organic disease of the gastrointestinal tract, but with one or more reproducible gastrointestinal symptoms that affect the quality of life of a subject, e.g., human by administering compounds as disclosed and described herein, individually or in combination to an individual in need thereof.

Exemplary functional gastrointestinal disorders include, without any limitation, functional dyspepsia, gastroesophageal reflux condition, diabetic gastroparesis, reflux esophagitis, postoperative gastrointestinal dysfunction and the like, nausea, vomiting, sickly feeling, heartburn, feeling of abdominal distention, heavy stomach, belching, chest writhing, chest pain, gastric discomfort, anorexia, dysphagia, reflux of gastric acid, abdominal pain, constipation, diarrhea, breathlessness, feeling of smothering, low incentive or energy level, pharyngeal obstruction, feeling of foreign substance, easy fatigability, stiff neck, myotonia, mouth dryness (dry mouth, thirst, etc.) tachypnea, burning sensation in the gastrointestinal tract, cold sensation of extremities, difficulty in concentration, impatience, sleep disorder, headache, general malaise, palpitation, night sweat, anxiety, dizziness, vertigo, hot flash, excess sweating, depression, etc.

In some embodiments, the method includes increasing or promoting digestion, absorption, blood nutrient level, and/or motility of gastrointestinal tract in a subject, e.g., promotion of gastric emptying (e.g., clearance of stomach contents), reduction of abdominal distention in the early postprandial period, improvement of anorexia, etc. by administering compounds as disclosed and described herein, individually or in combination to an individual in need thereof. In general, such promotion can be achieved either directly or via increasing the secretion of a regulatory entity, e.g., hormones, etc. by administering compounds as disclosed and described herein, individually or in combination to an individual in need thereof

In some embodiments, the method includes increasing one or more gastrointestinal functions of a subject, e.g., to improve the quality of life or healthy state of an individual by administering compounds as disclosed and described herein, individually or in combination.

Some embodiments provide a method for treating a respiratory tract infection including administering compounds as disclosed and described herein, individually or in combination to an individual in need thereof. In some embodiments, compounds as disclosed and described herein, individually or in combination can be used for inhibition of respiratory tract infections. Some embodiments provide a method for treating infertility including administering compounds as disclosed and described herein, individually or in combination to an individual in need thereof.

Some embodiments provide a pharmaceutical composition containing a therapeutically effective amount of one or more compounds as disclosed and described herein, or a salt, solvate, and/or prodrug thereof, optionally with a suitable amount of a pharmaceutically acceptable vehicle. In another embodiment, the pharmaceutical composition comprises a therapeutically effective amount of one or more compounds as disclosed and described herein, or a salt, solvate, and/or prodrug thereof; and a suitable amount of a pharmaceutically acceptable vehicle so as to provide the form for proper administration to a patient.

In one embodiment, when administered to a patient, the compounds as disclosed and described herein and the optional pharmaceutically acceptable vehicles are sterile. In one embodiment, water is a preferred vehicle when a compound as disclosed and described herein is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions comprising a compound as disclosed and described herein may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds of the present invention into preparations which can be used pharmaceutically. Proper formulation is dependent upon the route of administration chosen.

In some embodiments, the pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle is a capsule (see e.g., Grosswald et al., U.S. Pat. No. 5,698,155). Other examples of suitable pharmaceutical vehicles have been described in the art (see Remington: The Science and Practice of Pharmacy, Philadelphia College of Pharmacy and Science, 20th Edition, 2000).

For topical administration a compound as disclosed and described herein may be formulated as solutions, gels, ointments, creams, suspensions, etc. as is well-known in the art.

Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, but are not limited to, sodium channel blockers, antibiotics, N-acetyl cysteine, homocysteine and phospholipids.

In some embodiments, compounds as disclosed and described herein may be formulated in accordance with routine procedures as a pharmaceutical composition adapted for intravenous administration to human beings. Typically, compounds for intravenous administration are solutions in sterile isotonic aqueous buffer. For injection, a compound may be formulated in aqueous solutions, preferably in physiologically compatible buffers such as Hanks' solution, Ringer's solution, or physiological saline buffer. The solution may contain formulatory agents such as suspending, stabilizing and/or dispersing agents. When necessary, the pharmaceutical compositions may also include a solubilizing agent.

Pharmaceutical compositions for intravenous administration may optionally include a local anesthetic such as lignocaine to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. When a compound is administered by infusion, it can be dispensed, for example, with an infusion bottle containing sterile pharmaceutical grade water or saline. In some embodiments, an ampoule of sterile water for injection or saline can be provided so that the ingredients may be mixed prior to administration when a compound is administered by injection.

For transmucosal administration, penetrants appropriate to the barrier to be permeated are used in the formulation. Such penetrants are generally known in the art.

Pharmaceutical compositions for oral delivery may be in the form of tablets, lozenges, aqueous or oily suspensions, granules, powders, emulsions, capsules, syrups, or elixirs, for example. Orally administered pharmaceutical compositions may contain one or more optionally agents, for example, sweetening agents such as fructose, aspartame or saccharin; flavoring agents such as peppermint, oil of wintergreen, or cherry coloring agents and preserving agents, to provide a pharmaceutically palatable preparation.

Moreover, where in tablet or pill form, the pharmaceutical compositions may be coated to delay disintegration and absorption in the gastrointestinal tract, thereby providing a sustained action over an extended period of time. Selectively permeable membranes surrounding an osmotically active driving compound are also suitable for orally administered compounds of the present invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM) etc. Additionally, flavoring agents, preservatives, coloring agents, bile salts, acylcarnitines and the like may be added.

For buccal administration, the pharmaceutical compositions may take the form of tablets, lozenges, etc. formulated in conventional manner.

Liquid drug formulations suitable for use with nebulizers and liquid spray devices and EHD aerosol devices will typically include a compound of the present invention with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the invention. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

In some embodiments, a compound as disclosed and described herein may also be formulated in rectal or vaginal pharmaceutical compositions such as suppositories or retention enemas, e.g., containing conventional suppository bases such as cocoa butter or other glycerides.

In addition to the formulations described previously, a compound as disclosed and described herein may also be formulated as a depot preparation. Such long acting formulations may be administered by implantation (for example, subcutaneously or intramuscularly) or by intramuscular injection. Thus, for example, a compound of the present invention may be formulated with suitable polymeric or hydrophobic materials (for example, as an emulsion in an acceptable oil) or ion exchange resins, or as sparingly soluble derivatives, for example, as a sparingly soluble salt.

A compound as disclosed and described herein, and/or pharmaceutical composition thereof, will generally be used in an amount effective to achieve the intended purpose. For use to treat or prevent diseases or disorders the compounds as disclosed and described herein and/or pharmaceutical compositions thereof, are administered or applied in a therapeutically effective amount.

In some embodiments, the dosage may be delivered in a pharmaceutical composition by a single administration, by multiple applications or controlled release. In some embodiments, the compounds as disclosed and described herein may be delivered by oral sustained release administration. Dosing may be repeated intermittently, may be provided alone or in combination with other drugs and may continue as long as required for effective treatment of the disease state or disorder.

Suitable dosage ranges for oral administration depend on potency, but are generally between about 0.001 mg to about 200 mg of a compound as disclosed and described herein per kilogram body weight.

Suitable dosage ranges for intravenous (i.v.) administration are about 0.01 mg to about 100 mg per kilogram body weight. Suitable dosage ranges for intranasal administration are generally about 0.01 mg/kg body weight to about 1 mg/kg body weight. Suppositories generally contain about 0.01 milligram to about 50 milligrams of a compound of the present invention per kilogram body weight and comprise active ingredient in the range of about 0.5% to about 10% by weight. Recommended dosages for intradermal, intramuscular, intraperitoneal, subcutaneous, epidural, sublingual or intracerebral administration are in the range of about 0.001 mg to about 200 mg per kilogram of body weight. Effective doses may be extrapolated from dose-response curves derived from in vitro or animal model test systems.

In some embodiments, the dosage of a compound described herein will preferably be within a range of circulating concentrations that include an effective dose with little or no toxicity.

In certain embodiments, the compounds as disclosed and described herein and/or pharmaceutical compositions thereof can be used in combination therapy with at least one other agent. In some embodiments, a compound as disclosed and described herein and/or pharmaceutical composition thereof is administered concurrently with the administration of another agent, which may be part of the same pharmaceutical composition as the compound of the present invention or a different pharmaceutical composition. In other embodiments, a pharmaceutical composition of the present invention is administered prior or subsequent to administration of another agent.

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

Some exemplary synthetic methods for preparing the present compounds are illustrated in Scheme 1 below. Compounds of Formula VIII in Scheme 1 correspond to compounds of Formula (I) described elsewhere herein. PG are appropriate protecting groups. LG are appropriate leaving groups. EWD is an appropriate electron withdrawing group. R is any appropriate group to achieve the indicated reaction.

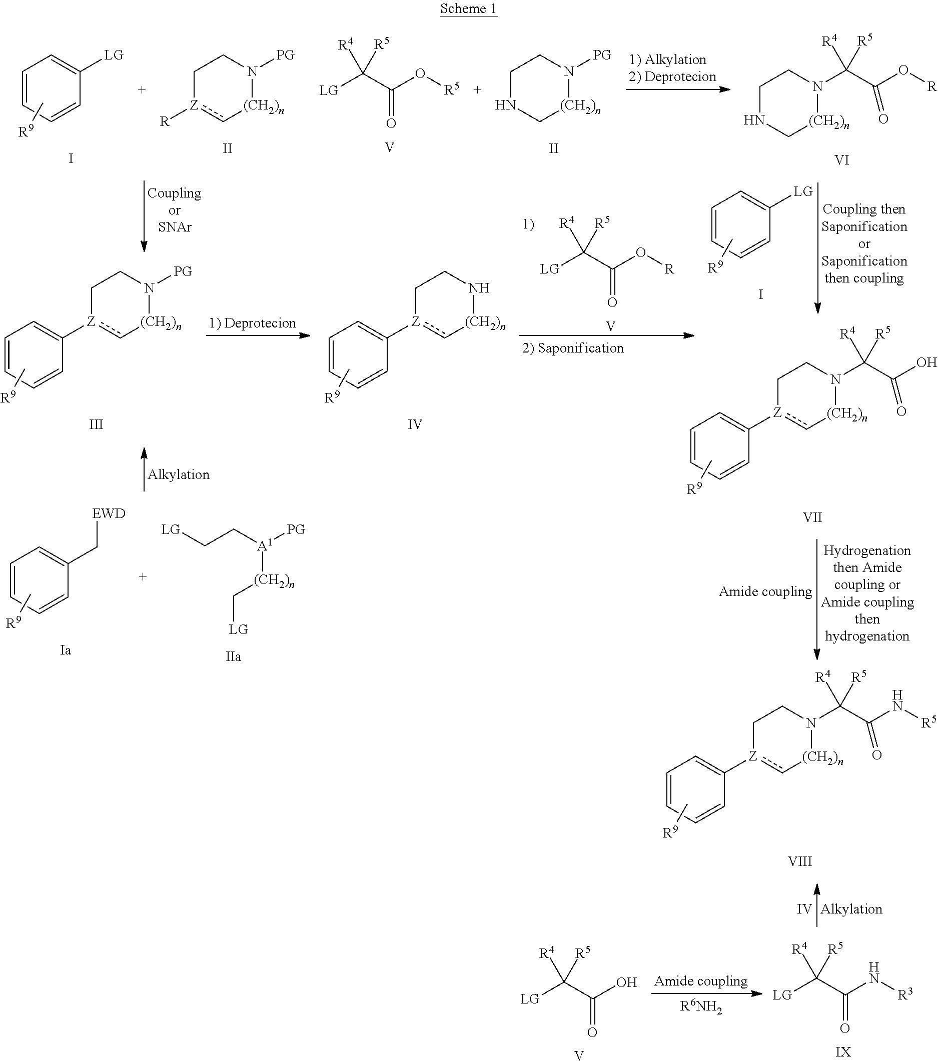

As shown in Scheme 1, building blocks I and II can be allowed to react under various coupling or SNAr conditions to yield III. Certain intermediates of formula III can also be synthesized by double alkylation of Ia with IIa. The removal of the protecting group of III would afford IV that can be converted by alkylation with V followed by saponification to yield VII. In some cases, V and II can be reacted first to yield VI followed by aromatic coupling and saponification (or vice versa) to yield the corresponding intermediate VII. Subsequently, VII can be reacted with various amines under amides coupling conditions to furnish the product VIII. Alternatively, V may be converted to IX, which in turn can serve as building block for direct alkylation of intermediate IV to yield VIII. If necessary the substituents can be modified as needed at any appropriate stage of the synthesis which may include the derivatization of VIII to form additional analogs.

### EXAMPLES

To further illustrate this invention, the following examples are included. The examples should not be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1: N-cyclopropyl-2-(4-(2,3-dimethylphenyl)piperazin-1-yl)-2-methylpropanamide (1)

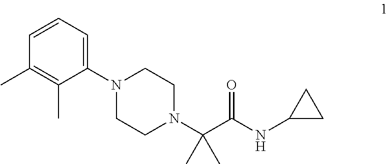 [SMILES: Cc1cccc(N2CCN(C(C)(C)C(=O)NC3CC3)CC2)c1C]

2-(4-(2,3-dimethylphenyl)piperazin-1-yl)-2-methylpropanoic acid (Example 1a) (552 mg, 2 mmol), EDC (382 mg, 2 mmol), HOBt (306 mg, 2 mmol), cyclopropylamine (114 mg, 2 mmol), and triethylamine (202 mg, 2 mmol) were suspended in DMF (15 mL) and irradiated in the microwave at 150° C. for 5 minutes. The reaction mixture was diluted with ethyl acetate and washed with water. The organic layer was collected and evaporated under vacuum. The crude product was dissolved in methanol and purified by reverse phase preparative HPLC and recrystallized from ethanol/water to provide the title compound (227 mg, 36%) as a white solid. ¹H NMR (DMSO-d₆) δ: 0.40-0.53 (m, 2H), 0.55-0.66 (m, 2H), 1.10 (s, 6H), 2.13 (s, 3H), 2.19 (s, 3H), 2.62 (m, 1H), 2.81 (t, J=4.6 Hz, 4H), 6.76-6.95 (m, 2H), 7.03 (t, J=7.7 Hz, 1H), 7.56 (d, J=4.4 Hz, 1H). MS 316 (M+H⁺).

### Example 1a: 2-(4-(2,3-dimethylphenyl)piperazin-1-yl)-2-methylpropanoic acid

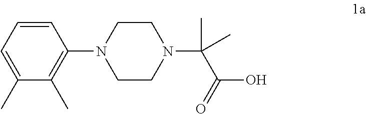 [SMILES: Cc1cccc(N2CCN(C(C)(C)C(=O)O)CC2)c1C]

To a suspension of 1-(2,3-dimethylphenyl)piperazine (1.9 g, 10 mmol) and potassium carbonate (1.6 g, 12 mmol) in DMF (7 mL), ethyl 2-bromo-2-methylpropanoate (2.3 g, 12 mmol) was added. The reaction was irradiated in the microwave at 150° C. for 5 minutes. The reaction mixture was diluted with ethyl acetate and was washed with water. The organic layer was collected, dried over sodium sulfate, and evaporated under vacuum. The crude was suspended in ethanol (5 mL) and 1 M LiOH (10 mL) was added. The reaction was irradiated in the microwave at 120° C. for 5 minutes. The reaction mixture was diluted with water and washed with ethyl acetate. The water layer was acidified to pH 6. The product was extracted using DCM/IPA (4:1) three times. The combined organic layer was dried over sodium sulfate and evaporated under vacuum to give the desired product. (950 mg, 34%) MS 277 (M+H⁺).

### Example 2: 2-(4-(2-fluorophenyl)piperazin-1-yl)-N-isobutyl-2-methylpropanamide (2)

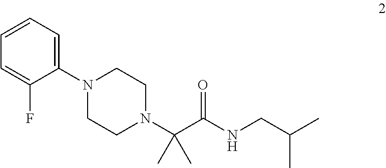 [SMILES: CC(C)CNC(=O)C(C)(C)N1CCN(c2ccccc2F)CC1]

1-(2-fluorophenyl)piperazine (540 mg, 3 mmol) and sodium hydride (70 mg, 3 mmol), were suspended in DMF (5 mL) and stirred at room temperature for 15 minutes. 2-Bromo-N-isobutyl-2-methylpropanamide (Example 2a) (663 mg, 3 mmol) was added and the reaction was irradiated in the microwave at 150° C. for 10 minutes. The reaction mixture was diluted with ethyl acetate and washed with water. The organic layer was dried over sodium sulfate and evaporated under vacuum. The crude product was purified by column chromatography (ethyl acetate/hexanes). The combined fractions was dried under vacuum, the residue dissolved in methanol and purified by reverse phase preparative HPLC and the product was lyophilized to provide the title compound (350 mg, 36%) as a white solid. ¹H NMR (DMSO-d₆) δ: 0.83 (d, J=6.7 Hz, 6H), 1.12 (s, 6H), 1.73 (hept, J=6.8 Hz, 1H), 2.57 (t, J=5.0 Hz, 4H), 2.84-2.94 (m, 2H), 3.07 (t, J=4.7 Hz, 4H), 6.92-6.99 (m, 1H), 6.99-7.06 (m, 1H), 7.07-7.16 (m, 2H), 7.72 (t, J=6.2 Hz, 1H). MS 322 (M+H⁺).

### Example 2a: 2-bromo-N-isobutyl-2-methylpropanamide

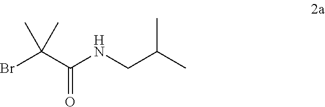

A suspension of 2-bromo-2-methylpropanoic acid (8.35 g, 50 mmol), TBTU (16 g, 50 mmol) and triethylamine (7 mL, 50 mmol) in DCM (250 mL) was stirred at room temperature for 30 minutes followed by the addition of 2-methylpropan-1-amine (5 mL, 50 mmol). The reaction was stirred at room temperature overnight. The reaction mixture was diluted with DCM and washed with water. The organic layer was dried over sodium sulfate, and evaporated under vacuum. The crude material was purified by column chromatography (ethyl acetate/hexanes) to give the desired product as a white solid (7 g, 63%). ¹H NMR (DMSO-d₆) δ 0.83 (d, J=6.7 Hz, 6H), 1.68-1.83 (m, 1H), 1.86 (s, 6H), 2.90 (dd, J=7.1, 5.9 Hz, 2H), 8.06 (br, 1H).

### Example 3: 2-(4-(2,3-difluorophenyl)piperazin-1-yl)-N-isobutyl-2-methylpropanamide (3)

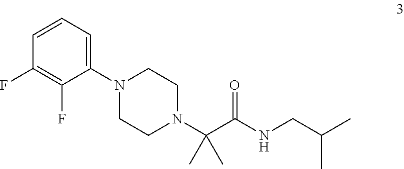 [SMILES: CC(C)CNC(=O)C(C)(C)N1CCN(c2cccc(F)c2F)CC1]

A mixture of 2-(4-(2,3-difluorophenyl)piperazin-1-yl)-2-methylpropanoic acid (Example 3a) (320 mg, 1.13 mmol), TBTU (433 mg. 1.36 mmol), DIEA (392 uL, 2.25 mmol) and 2-methylpropan-1-amine (136 uM, 1.36 mmol) in DCM (25 mL) was stirred at room temperature overnight. The resulting solution was concentrated, the residue was purified by mass-triggered reverse phase preparative HPLC (Acetonitrile/Water) and lyophilized to obtain the title compound (290 mg, yield 75%) as a beige solid. ¹H NMR (DMSO-d₆, 400 MHz) δ: 0.81 (d, J=6.4 Hz, 6H), 1.09 (s, 6H), 1.71 (m, 1H), 2.55 (t, J=4.8 Hz, 4H), 2.89 (t, J=6 Hz, 2H), 2.09 (t, J=4.4 Hz, 4H), 6.83 (m, 1H), 6.96 (m, 1H), 7.65 (m, 1H), 7.71 (t, J=6 Hz, 1H). MS 340 (M+H⁺).

### Example 3a: 2-(4-(2,3-difluorophenyl)piperazin-1-yl)-2-methylpropanoic acid

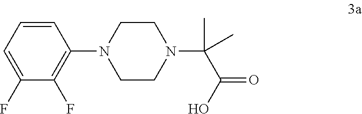 [SMILES: CC(C)(C(=O)O)N1CCN(c2cccc(F)c2F)CC1]

A suspension of 2-methyl-2-(piperazin-1-yl)propanoic acid dihydrochloride salt (Example 3b) (1.23 g, 5.0 mmol), 1-bromo-2,3-difluorobenzene (572 uL, 5.0 mmol), BINAP (311 mg, 0.5 mmol), Pd(dba)₂ (58 mg, 0.1 mmol), and sodium tert-butoxide (1.92 g, 20 mmol) in toluene (40 mL) in a pressure vessel was heated at 150° C. for 18 hours. The reaction mixture was cooled to room temperature, concentrated under vacuum, and the residue diluted with water (50 mL), and extracted with MTBE (15 mL×3). The aqueous layer was acidified to pH=7, and product was extracted out using EtOAc/n-butanol (5:1). The organic layers were collected, and dried over MgSO₄, the solvent was evaporated to obtain 320 mg of desired product as an off white solid (22% yield). MS 285 (M+H⁺).

### Example 3b: 2-methyl-2-(piperazin-1-yl)propanoic acid dihydrochloride salt

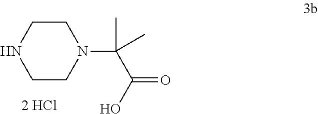

2-(4-(tert-butoxycarbonyl)piperazin-1-yl)-2-methylpropanoic acid intermediate (Example 3c) (2 g, 7.3 mmol) was suspended in methanol (20 mL) and HCl in methanol was added. The reaction was stirred at room temperature for 30 minutes and concentrated down under vacuum to give the desired compound as a light yellow solid. (1.8 g, 100%).

### Example 3c: 2-(4-(tert-butoxycarbonyl)piperazin-1-yl)-2-methylpropanoic acid

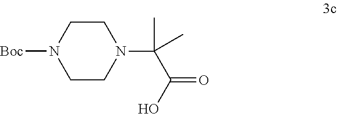 [SMILES: CC(C)(C)OC(=O)N1CCN(C(C)(C)C(=O)O)CC1]

To a suspension of Boc-piperazine (18.6 g, 100 mmol) and potassium carbonate (27.4 g, 200 mmol) in acetonitrile (250 mL) was added methyl 2-bromo-2-methylpropanoate (15.5 mL, 120 mmol) dropwise at room temperature. The reaction was subsequently heated at 83° C. overnight, cooled down to room temperature, diluted with ethyl acetate, and washed with water. The organic layer was dried over sodium sulfate and evaporated under vacuum to give a clear liquid which was dissolved in methanol (100 mL). 1M NaOH (150 mL) was added and the reaction mixture was heated at 85° C. for 3 hours, diluted with water, and washed with ethyl acetate. The aqueous layer was acidified to pH ˜5 and concentrated under vacuum. The crude material was suspended in dry ethanol, stirred for 30 minutes, and filtered. The filtrate was collected and evaporated under vacuum to give the intermediate 2-(4-(tert-butoxycarbonyl)piperazin-1-yl)-2-methylpropanoic acid as an off white solid (12.7 g, 46%). MS 273 (M+H⁺).

### Example 4: 2-(4-(2-fluoro-3-(methoxymethyl)phenyl)piperazin-1-yl)-N-isobutyl-2-methylpropanamide (4)

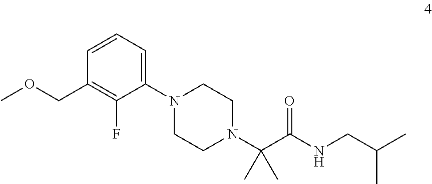 [SMILES: COCc1cccc(N2CCN(C(C)(C)C(=O)NCC(C)C)CC2)c1F]

A suspension of 1-bromo-2-fluoro-3-(methoxymethyl)benzene (350 mg, 1.6 mmol), N-isobutyl-2-methyl-2-(piperazin-1-yl)propanamide dihydrochloride salt (Example 4a) (500 mg, 1.6 mmol), BINAP (99 mg, 0.16 mmol), Pd₂(dba)₃ (30 mg, 0.032 mmol), and sodium tert-butoxide (614 mg, 6.4 mmol) in toluene (20 mL) was stirred at 85° C. overnight. The reaction mixture was diluted with ethyl acetate and washed with water. The organic layer was dried over sodium sulfate, concentrated, and purified by column chromatography (ethyl acetate/hexanes). The combined fractions were further purified on HPLC (acetonitrile/water) and the product was lyophilized to give a white solid (250 mg, 42%). ¹H NMR (DMSO-d₆) δ 0.83 (d, J=6.7 Hz, 6H), 1.12 (s, 6H), 1.73 (hept, J=6.8 Hz, 1H), 2.56 (t, J=4.6 Hz, 4H), 2.92 (t, J=6.5 Hz, 2H), 3.06 (d, J=4.9 Hz, 4H), 3.28 (s, 3H), 4.42 (d, J=1.4 Hz, 2H), 6.98 (t, J=7.2 Hz, 2H), 7.72 (t, J=6.2 Hz, 1H). MS 366 (MH+).

### Example 4a: N-isobutyl-2-methyl-2-(piperazin-1-yl)propanamide dihydrochloride salt

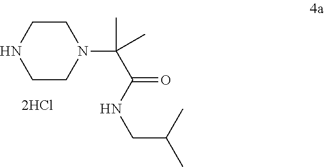 [SMILES: CC(C)CNC(=O)C(C)(C)N1CCNCC1]

To a solution of 2-(4-(tert-butoxycarbonyl)piperazin-1-yl)-2-methylpropanoic acid (Example 3c) (5.5 g, 20.2 mmol) in dry DCM (200 mL) was added HATU (7.6 g, 20.2 mmol), TEA (2.9 mL, 20.2 mmol), and isobutylamine (2.1 mL, 20.2 mmol). The solution was stirred at room temperature for 16 hours. The precipitates were filtered off and the filtrate was diluted with DCM and washed with H₂O. The organic layer was dried over Na₂SO₄ and concentrated to afford tert-butyl 4-(1-(isobutylamino)-2-methyl-1-oxopropan-2-yl)piperazine-1-carboxylate as a yellow oil. The crude residue was dissolved in dioxane (20 mL) and 4N HCl in dioxane (10 mL) was added. The reaction was stirred at room temperature for 16 hours and then concentrated to afford the desired compound as a peach colored solid (6.1 g, quantitative yield).

### Example 5: 2-(4-(3-fluoro-2-(methoxymethyl)phenyl)piperazin-1-yl)-N-isobutyl-2-methylpropanamide (5)

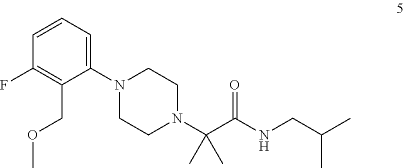 [SMILES: COCc1c(F)cccc1N1CCN(C(C)(C)C(=O)NCC(C)C)CC1]

To a solution of 2-(4-(3-fluoro-2-(hydroxymethyl)phenyl)piperazin-1-yl)-N-isobutyl-2-methylpropanamide (Example 5a) (150 mg, 0.427 mmol) in dry DMF (2 mL) at 0° C. was added NaH (60% wt in mineral oil, 22 mg, 0.555 mmol) and the solution was stirred at the same temperature for 1 hour. Iodomethane (28 uL, 0.448 mmol) was added and the solution was stirred at room temperature for 16 hours. The solution was placed on an ice bath and quenched with a few drops of MeOH while stirring. The solution was filtered through a syringe filter and purified by HPLC (water/ACN gradient). The clean fractions were concentrated on the lyophilizer overnight to afford the title compound as a white powder (81.1 mg, 52% yield). ¹H NMR (400 MHz, d₆-DMSO) δ 0.84 (d, J=6.8 Hz, 6H), 1.13 (s, 6H), 1.75 (m, 1H), 2.59 (br. s, 4H), 2.92 (t, J=6.4 Hz, 2H), 3.01 (br. s, 4H), 3.30 (s, 3H), 4.41 (d, J=1.6 Hz, 2H), 6.86-6.91 (m, 2H), 7.32 (q, J=8.4 Hz, 1H), 7.70 (t, J=6.4 Hz, 1H). MS 366 (M+H⁺).

### Example 5a: 2-(4-(3-fluoro-2-(hydroxymethyl)phenyl)piperazin-1-yl)-N-isobutyl-2-methylpropanamide (51)

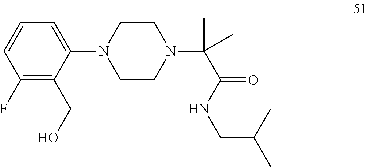 [SMILES: CC(C)CNC(=O)C(C)(C)N1CCN(c2cccc(F)c2CO)CC1]

To a solution of methyl 2-fluoro-6-(4-(1-(isobutylamino)-2-methyl-1-oxopropan-2-yl)piperazin-1-yl)benzoate (Example 5b) (155 mg, 0.408 mmol) in dry THF (8 mL) at 0° C. was added LiAlH₄ (46 mg, 0.612 mmol) in two portions and the reaction was stirred at the same temperature for 30 minutes. The ice bath was removed and the reaction was stirred at room temperature for 3.5 hours, cooled back down to 0° C. and quenched successively with water (300 uL), 15% NaOH (300 uL), and water (900 uL). The solution was stirred at room temperature for 16 hours. The solids were filtered off, washed with EtOAc, and the resulting filtrate was concentrated down to afford 2-(4-(3-fluoro-2-(hydroxymethyl)phenyl)piperazin-1-yl)-N-isobutyl-2-methylpropanamide as a yellow oil (150 mg, quantitative yield).

### Example 5b: Methyl 2-fluoro-6-(4-(1-(isobutylamino)-2-methyl-1-oxopropan-2-yl)piperazin-1-yl)benzoate (7)

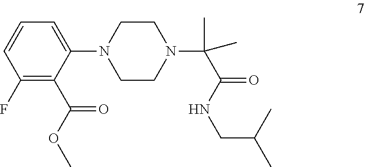 [SMILES: COC(=O)c1c(F)cccc1N1CCN(C(C)(C)C(=O)NCC(C)C)CC1]

A solution of N-isobutyl-2-methyl-2-(piperazin-1-yl)propanamide HCl salt (Example 4a) (650 mg, 2.17 mmol), methyl 2,6-difluorobenzoate (291 uL, 2.17 mmol), K₂CO₃ (898 mg, 6.50 mmol) in dry DMF (10 mL) was heated at 90° C. for 48 hours. The reaction was diluted with EtOAc and washed with H₂O. The organic layer was dried over Na₂SO₄ and concentrated. The crude residue was purified by silica gel chromatography using a 0-100% EtOAc/Hexanes gradient. The clean fractions were concentrated to afford the desired compound as a colorless oil (155 mg, 19% yield). ¹H NMR (400 MHz, d₆-DMSO) δ 0.84 (d, J=6.8 Hz, 6H), 1.10 (s, 6H), 1.73 (m, 1H), 2.47 (br. s, 4H), 2.91 (t, J=6.4 Hz, 2H), 3.02 (t, J=4.8 Hz, 4H), 3.83 (s, 3H), 6.89-6.96 (m, 2H), 7.43 (q, J=8.4 Hz, 1H), 7.70 (t, J=6.0 Hz, 1H). MS 380 (M+H⁺).

Compounds in Table 1 were prepared in a similar manner as in Example 1, Example 2, Example 3, Example 4, and/or Example 5 from the corresponding commercially available building blocks.

TABLE 1
Structures and data for selected compounds.

| | | Observed | Sweet | |
|---|---|---|---|---|
| | | Molecular Ion | EC₅₀ | |
| Cpd # | Structure | MS (M + 1) | (μM) | ¹H NMR (400 MHz) |
| 8 | 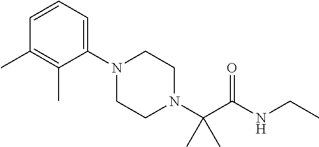 [SMILES: CCNC(=O)C(C)(C)N1CCN(c2cccc(C)c2C)CC1] | 304 | >10 | |
| 9 | 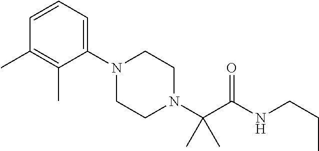 [SMILES: CCCNC(=O)C(C)(C)N1CCN(c2cccc(C)c2C)CC1] | 318 | 4.2 | (DMSO-d₆) δ 0.86 (t, J = 7.4 Hz, 3H), 1.50 (m, 2H), 1.58 (s, 6H), 2.17 (s, 3H), 2.22 (s, 3H), 2.54 (s, 4H), 3.08-3.19 (m, 6H), 6.93 (t, J = 8.5 Hz, 2H), 7.09 (t, J = 7.7 Hz, 1H), 8.36 (t, J = 5.6 Hz, 1H). |
| 10 | 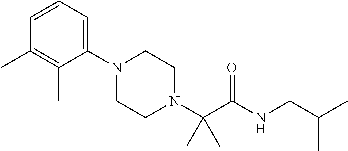 [SMILES: Cc1cccc(N2CCN(C(C)(C)C(=O)NCC(C)C)CC2)c1C] | 332 | 2.8 | (DMSO-d₆) δ 0.85 (dd, J = 13.2, 6.7 Hz, 6H), 1.12 (s, 6H), 1.73 (m, 1H), 2.18 (d, J = 20, 6H), 2.57 (m, 4H), 2.85 (m, 4H), 2.92 (t, J = 6.5 Hz, 2H), 6.90 (m, 2H), 7.00-7.11 (m, 1H), 7.71 (t, J = 6.1 Hz, 1H). |
| 11 | 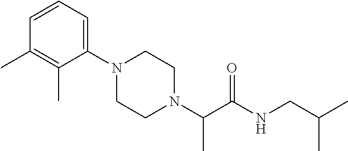 [SMILES: Cc1cccc(N2CCN(C(C)C(=O)NCC(C)C)CC2)c1C] | 318 | 1.4 | (DMSO-d₆) δ 0.84 (dd, J = 6.7, 0.6 Hz, 6H), 1.13 (d, J = 6.9 Hz, 3H), 1.73 (m, 1H), 2.13 (s, 3H), 2.19 (s, 3H), 2.61 (m, 4H), 2.80 (m, 4H), 2.92 (m, 2H), 3.07 (m, 1H), 6.87 (t, J = 7.6 Hz, 2H), 7.03 (t, J = 7.7 Hz, 1H), 7.79 (t, J = 6.1 Hz, 1H). |
| 12 | 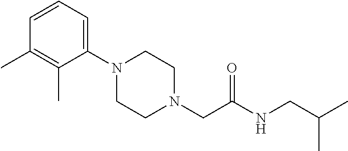 [SMILES: Cc1cccc(N2CCN(CC(=O)NCC(C)C)CC2)c1C] | 304 | 9.7 | (DMSO-d₆) δ 0.84 (d, J = 6.7 Hz, 5H), 1.72 (m, 1H), 2.14 (s, 3H), 2.20 (s, 3H), 2.66 (m, 4H), 2.85 (m, 4H), 2.94 (t, J = 6.5 Hz, 2H), 6.88 (t, J = 7.8 Hz, 2H), 7.04 (t, J = 7.7 Hz, 1H), 7.80 (br, 1H). |
| 13 | 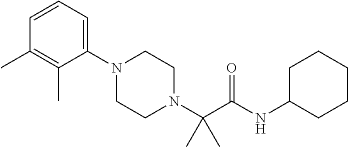 [SMILES: Cc1cccc(N2CCN(C(C)(C)C(=O)NC3CCCCC3)CC2)c1C] | 358 | 9.4 | |
| 14 | 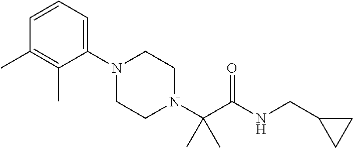 [SMILES: Cc1cccc(N2CCN(C(C)(C)C(=O)NCC3CC3)CC2)c1C] | 330 | 5 | (DMSO-d₆) δ 0.13-0.20 (m, 2H), 0.32- 0.40 (m, 2H), 0.96 (s, 1H), 1.12 (s, 6H), 2.14 (s, 3H), 2.20 (s, 3H), 2.58 (s, 4H), 2.74-2.93 (m, 6H), 2.98 (t, J = 6.4 Hz, 2H), 6.88 (m, 2H), 7.04 (m, 1H), 7.72 (t, J = 6.0 Hz, 1H). |
| 15 | 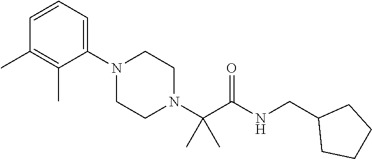 [SMILES: Cc1cccc(N2CCN(C(C)(C)C(=O)NCC3CCCC3)CC2)c1C] | 358 | 6.9 | (DMSO-d₆) δ 1.12 (s, 6H), 1.38-1.67 (m, 8H), 2.01-2.07 (m, 1H), 2.14 (s, 3H), 2.20 (s, 3H), 2.56 (m, 4H), 2.84 (m, 4H), 3.02 (dd, J = 7.2, 6.1 Hz, 2H), 6.87 (t, J = 8.2 Hz, 2H), 7.04 (t, J = 7.7 Hz, 1H), 7.71 (t, J = 6.0 Hz, 1H). |
| 16 | 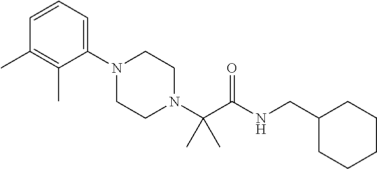 [SMILES: Cc1cccc(N2CCN(C(C)(C)C(=O)NCC3CCCCC3)CC2)c1C] | 372 | >10 | |
| 17 | 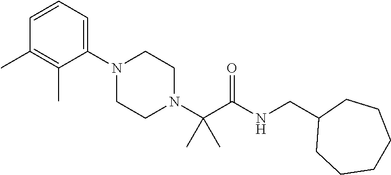 [SMILES: Cc1cccc(N2CCN(C(C)(C)C(=O)NCC3CCCCCC3)CC2)c1C] | 386 | >10 | |
| 18 | 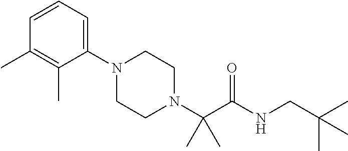 [SMILES: Cc1cccc(N2CCN(C(C)(C)C(=O)NCC(C)(C)C)CC2)c1C] | 346 | >10 | |
| 19 | 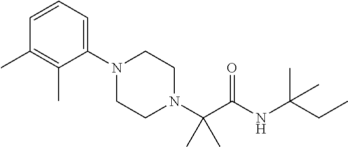 [SMILES: CCC(C)(C)NC(=O)C(C)(C)N1CCN(c2cccc(C)c2C)CC1] | 346 | >10 | |
| 20 | 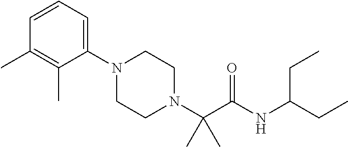 [SMILES: CCC(CC)NC(=O)C(C)(C)N1CCN(c2cccc(C)c2C)CC1] | 346 | 5.6 | (DMSO-d₆) δ 0.80 (td, J = 7.4, 2.1 Hz, 6H), 1.14 (d, J = 2.0 Hz, 6H), 1.29-1.52 (m, 5H), 2.17 (dd, J = 20.9, 2.0 Hz, 6H), 2.58 (m, 4H), 2.85 (m, 4H), 6.83-6.92 (m, 2H), 7.05 (m, 1 H), 7.28 (m, 1H) |
| 21 | 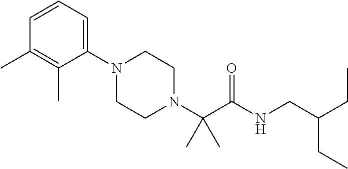 [SMILES: CCC(CC)CNC(=O)C(C)(C)N1CCN(c2cccc(C)c2C)CC1] | 360 | >10 | |
| 22 | 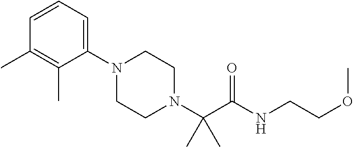 | 334 | >10 | |
| 23 | 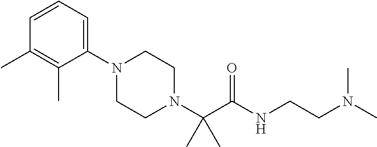 | 347 | >10 | |
| 24 | 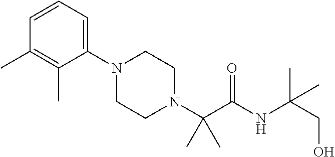 [SMILES: Cc1cccc(N2CCN(C(C)(C)C(=O)NC(C)(C)CO)CC2)c1C] | 348 | >10 | |
| 25 | 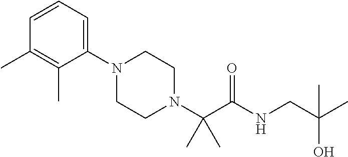 [SMILES: Cc1cccc(N2CCN(C(C)(C)C(=O)NCC(C)(C)O)CC2)c1C] | 348 | >10 | |
| 26 | 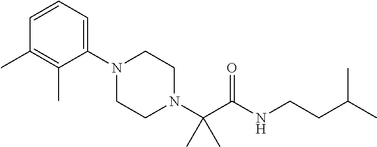 [SMILES: Cc1cccc(N2CCN(C(C)(C)C(=O)NCCC(C)C)CC2)c1C] | 346 | 8 | |
| 27 | 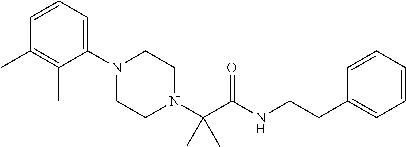 [SMILES: Cc1cccc(N2CCN(C(C)(C)C(=O)NCCc3ccccc3)CC2)c1C] | 380 | >10 | |
| 28 | 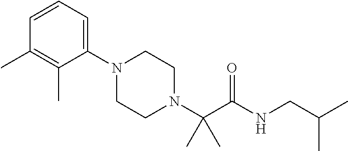 [SMILES: Cc1cccc(N2CCN(C(C)(C)C(=O)NCC(C)C)CC2)c1C] | 332 | 2.8 | (DMSO-d₆) δ 0.85 (dd, J = 13.2, 6.7 Hz, 6H), 1.12 (s, 6H), 1.73 (m, 1H), 2.18 (d, J = 20, 6H), 2.57 (m, 4H), 2.85 (m, 4H), 2.92 (t, J = 6.5 Hz, 2H), 6.90 (m, 2H), 7.00-7.11 (m, 1H), 7.71 (t, J = 6.1 Hz, 1H). |
| 29 | 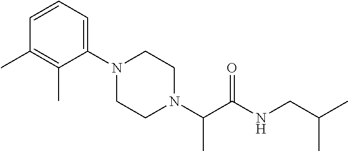 [SMILES: Cc1cccc(N2CCN(C(C)C(=O)NCC(C)C)CC2)c1C] | 318 | 1.4 | (DMSO-d₆) δ 0.84 (dd, J = 6.7, 0.6 Hz, 6H), 1.13 (d, J = 6.9 Hz, 3H), 1.73 (m, 1H), 2.13 (s, 3H), 2.19 (s, 3H), 2.61 (m, 4H), 2.80 (m, 4H), 2.92 (m, 2H), 3.07 (m, 1H), 6.87 (t, J = 7.6 Hz, 2H), 7.03 (t, J = 7.7 Hz, 1H), 7.79 (t, J = 6.1 Hz, 1H). |
| 30 | 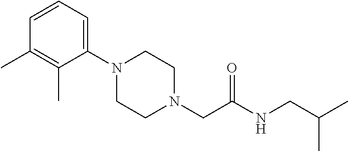 [SMILES: Cc1cccc(N2CCN(CC(=O)NCC(C)C)CC2)c1C] | 304 | 9.7 | (DMSO-d₆) δ 0.84 (d, J = 6.7 Hz, 5H), 1.72 (m, 1H), 2.14 (s, 3H), 2.20 (s, 3H), 2.66 (m, 4H), 2.85 (m, 4H), 2.94 (t, J = 6.5 Hz, 2H), 6.88 (t, J = 7.8 Hz, 2H), 7.04 (t, J = 7.7 Hz, 1H), 7.80 (br, 1H). |
| 31 | 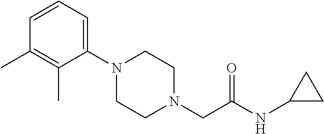 [SMILES: Cc1cccc(N2CCN(CC(=O)NC3CC3)CC2)c1C] | 288 | >10 | |
| 32 | 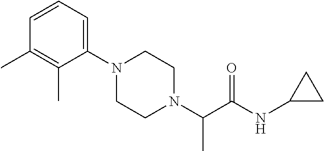 [SMILES: Cc1cccc(N2CCN(C(C)C(=O)NC3CC3)CC2)c1C] | 302 | >10 | (DMSO-d₆) δ 0.39-0.50 (m, 2H), 0.58 0.68 (m, 2H), 1.11 (d, J = 6.9 Hz, 3H), 2.13 (s, 3H), 2.19 (s, 3H), 2.53-2.71 (m, 5H), 2.78 (t, J = 4.7 Hz, 4H), 2.99 (q, J = 6.8 Hz, 1H), 6.86 (t, J = 8.0 Hz, 2H), 7.02 (t, J = 7.7 Hz, 1H), 7.79 (d, J = 4.4 Hz, 1H). |
| 1 | 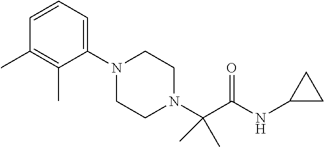 [SMILES: Cc1cccc(N2CCN(C(C)(C)C(=O)NC3CC3)CC2)c1C] | 316 | 4.3 | (DMSO-d₆) δ 0.40-0.53 (m, 2H), 0.55- 0.66 (m, 2H), 1.10 (s, 6H), 2.13 (s, 3H), 2.19 (s, 3H), 2.62 (m, 1H), 2.81 (t, J = 4.6 Hz, 4H), 6.76-6.95 (m, 2H), 7.03 (t, J = 7.7 Hz, 1H), 7.56 (d, J = 4.4 Hz, 1H). |
| 33 | 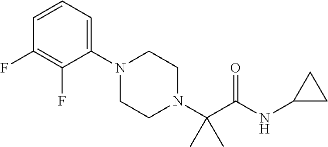 [SMILES: CC(C)(C(=O)NC1CC1)N1CCN(c2cccc(F)c2F)CC1] | 324 | 3.9 | (DMSO-d₆) δ 0.42-0.49 (m, 2H), 0.57- 0.66 (m, 2H), 1.09 (s, 6H), 2.51-2.54 (m, 4H), 2.62 (m, 1H), 3.08 (m, 4H), 6.85 (m, 1H), 6.97 (m, 1H), 7.09 (m, 1H), 7.59 (d, J = 4.4 Hz, 1H). |
| 34 | 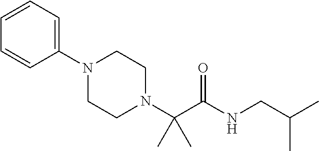 [SMILES: CC(C)CNC(=O)C(C)(C)N1CCN(c2ccccc2)CC1] | 304 | >10 | |
| 35 | 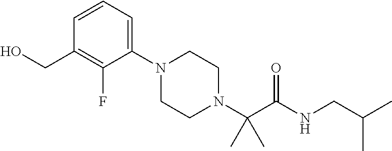 [SMILES: CC(C)CNC(=O)C(C)(C)N1CCN(c2cccc(CO)c2F)CC1] | 352 | >10 | |
| 36 | 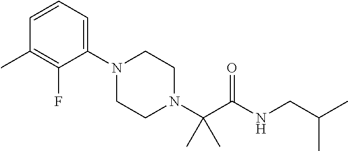 [SMILES: Cc1cccc(N2CCN(C(C)(C)C(=O)NCC(C)C)CC2)c1F] | 336 | 2.4 | (DMSO-d₆) δ 0.83 (d, J = 6.7 Hz, 6H), 1.11 (s, 6H), 1.73 (m, 1H), 2.19 (d, J = 2.3 Hz, 3H), 2.53-2.60 (m, 4H), 2.91 (t, J = 6.5 Hz, 2H), 3.04 (m, 4H), 6.85 (t, J = 7.7 Hz, 2H), 6.98 (dd, J = 8.5, 7.0 Hz, 1H), 7.72 (t, J = 6.2 Hz, 1H). |
| 3 | 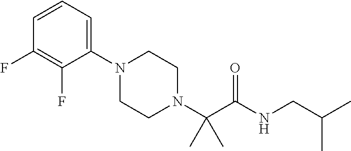 [SMILES: CC(C)CNC(=O)C(C)(C)N1CCN(c2cccc(F)c2F)CC1] | 340 | 1.6 | (DMSO-d₆) δ 0.81 (d, J = 6.4 Hz, 6H), 1.09 (s, 6H), 1.71 (m, 1H), 2.55 (t, J = 4.8 Hz, 4H), 2.89 (t, J = 6 Hz, 2H), 2.09 (t, J = 4.4 Hz, 4H), 6.83 (m, 1H), 6.96 (m, 1H), 7.65 (m, 1H), 7.71 (t, J = 6 Hz, 1H). |
| 37 | 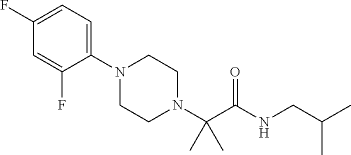 [SMILES: CC(C)CNC(=O)C(C)(C)N1CCN(c2ccc(F)cc2F)CC1] | 340 | >10 | |
| 38 | 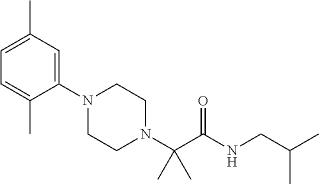 [SMILES: Cc1ccc(C)c(N2CCN(C(C)(C)C(=O)NCC(C)C)CC2)c1] | 332 | >10 | |
| 39 | 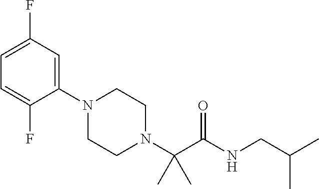 [SMILES: CC(C)CNC(=O)C(C)(C)N1CCN(c2cc(F)ccc2F)CC1] | 340 | >10 | |
| 40 | 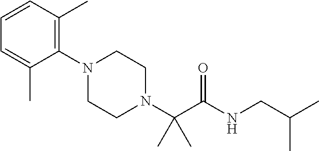 [SMILES: Cc1cccc(C)c1N1CCN(C(C)(C)C(=O)NCC(C)C)CC1] | 332 | >10 | |
| 41 | 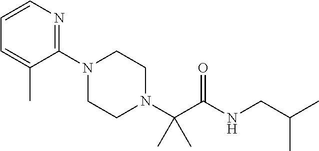 [SMILES: Cc1cccnc1N1CCN(C(C)(C)C(=O)NCC(C)C)CC1] | 319 | >10 | |
| 42 | 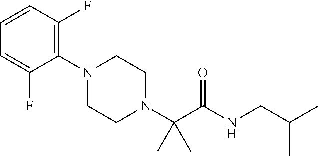 [SMILES: CC(C)CNC(=O)C(C)(C)N1CCN(c2c(F)cccc2F)CC1] | 340 | >10 | |
| 43 | 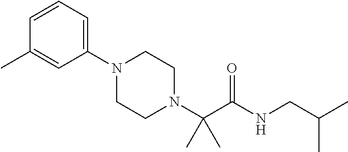 [SMILES: Cc1cccc(N2CCN(C(C)(C)C(=O)NCC(C)C)CC2)c1] | 318 | >10 | |
| 44 | 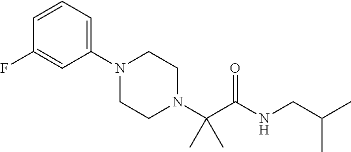 [SMILES: CC(C)CNC(=O)C(C)(C)N1CCN(c2cccc(F)c2)CC1] | 322 | >10 | |
| 2 | 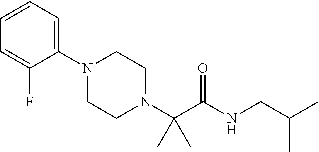 [SMILES: CC(C)CNC(=O)C(C)(C)N1CCN(c2ccccc2F)CC1] | 322 | 6.8 | (DMSO-d₆) δ 0.83 (d, J = 6.7 Hz, 6H), 1.12 (s, 6H), 1.73 (hept, J = 6.8 Hz, 1H), 2.57 (t, J = 5.0 Hz, 4H), 2.84-2.94 (m, 2H), 3.07 (t, J = 4.7 Hz, 4H), 6.92-6.99 (m, 1H), 6.99-7.06 (m, 1H), 7.07-7.16 (m, 2H), 7.72 (t, J = 6.2 Hz, 1H). |
| 45 | 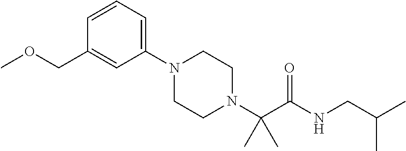 [SMILES: COCc1cccc(N2CCN(C(C)(C)C(=O)NCC(C)C)CC2)c1] | 348 | >10 | |
| 4 | 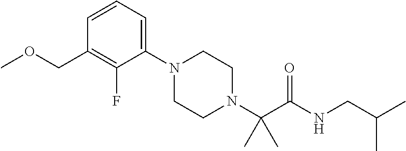 [SMILES: COCc1cccc(N2CCN(C(C)(C)C(=O)NCC(C)C)CC2)c1F] | 366 | 2.1 | (DMSO-d₆) δ 0.83 (d, J = 6.7 Hz, 6H), 1.12 (s, 6H), 1.73 (hept, J = 6.8 Hz, 1H), 2.56 (t, J = 4.6 Hz, 4H), 2.92 (t, J = 6.5 Hz, 2H), 3.06 (d, J = 4.9 Hz, 4H), 3.28 (s, 3H), 4.42 (d, J = 1.4 Hz, 2H), 6.98 (t, J = 7.2 Hz, 2H), 7.72 (t, J = 6.2 Hz, 1H). |
| 46 | 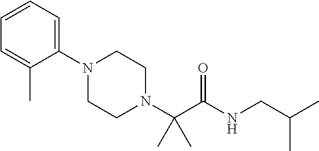 [SMILES: Cc1ccccc1N1CCN(C(C)(C)C(=O)NCC(C)C)CC1] | 318 | 4.3 | |
| 47 | 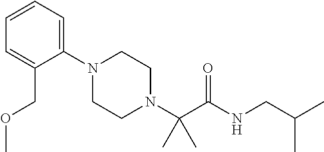 [SMILES: COCc1ccccc1N1CCN(C(C)(C)C(=O)NCC(C)C)CC1] | 348 | 6.8 | |
| 5 | 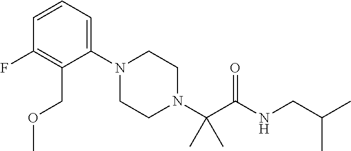 [SMILES: COCc1c(F)cccc1N1CCN(C(C)(C)C(=O)NCC(C)C)CC1] | 366 | 2.1 | (DMSO-d₆) δ 0.84 (d, J = 6.8 Hz, 6H), 1.13 (s, 6H), 1.75 (m, 1H), 2.59 (br. s, 4H), 2.92 (t, J = 6.4 Hz, 2H), 3.01 (br. s, 4H), 3.30 (s, 3H), 4.41 (d, J = 1.6 Hz, 2H), 6.86-6.91 (m, 2H), 7.32 (q, J = 8.4 Hz, 1H), 7.70 (t, J = 6.4 Hz, 1H). |
| 48 | 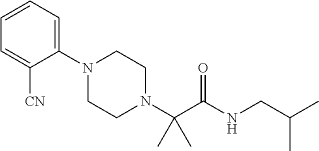 [SMILES: CC(C)CNC(=O)C(C)(C)N1CCN(c2ccccc2C#N)CC1] | 329 | >10 | |
| 49 | 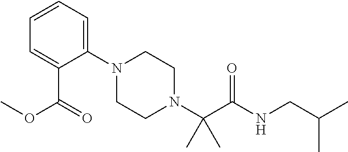 [SMILES: COC(=O)c1ccccc1N1CCN(C(C)(C)C(=O)NCC(C)C)CC1] | 362 | >10 | |
| 7 | 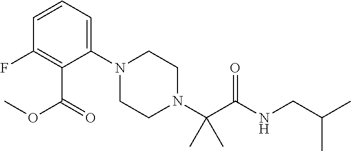 [SMILES: COC(=O)c1c(F)cccc1N1CCN(C(C)(C)C(=O)NCC(C)C)CC1] | 380 | 11.5 | (DMSO-d₆) δ 0.84 (d, J = 6.8 Hz, 6H), 1.10 (s, 6H), 1.73 (m, 1H), 2.47 (br. s, 4H), 2.91 (t, J = 6.4 Hz, 2H), 3.02 (t, J = 4.8 Hz, 4H), 3.83 (s, 3H), 6.89-6.96 (m, 2H), 7.43 (q, J = 8.4 Hz, 1H), 7.70 (t, J = 6.0 Hz, 1H). |
| 50 | 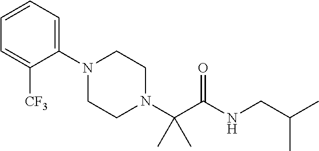 [SMILES: CC(C)CNC(=O)C(C)(C)N1CCN(c2ccccc2C(F)(F)F)CC1] | 372 | >10 | |
| 6 | 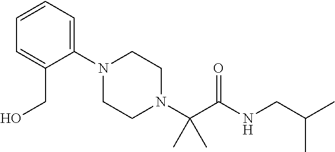 [SMILES: CC(C)CNC(=O)C(C)(C)N1CCN(c2ccccc2CO)CC1] | 334 | >10 | |
| 51 | 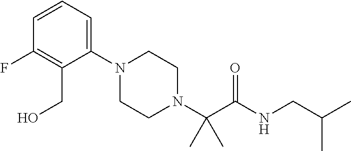 [SMILES: CC(C)CNC(=O)C(C)(C)N1CCN(c2cccc(F)c2CO)CC1] | 352 | >10 | |
| 52 | 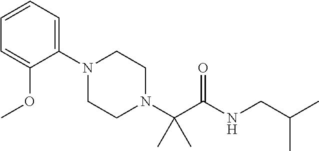 [SMILES: COc1ccccc1N1CCN(C(C)(C)C(=O)NCC(C)C)CC1] | 334 | >10 | |
| 53 | 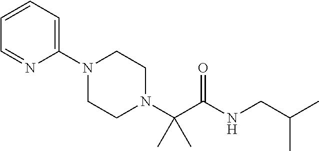 [SMILES: CC(C)CNC(=O)C(C)(C)N1CCN(c2ccccn2)CC1] | 305 | >10 | |
| 54 | 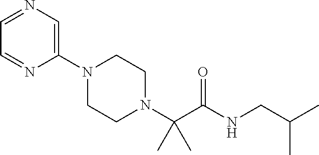 [SMILES: CC(C)CNC(=O)C(C)(C)N1CCN(c2cnccn2)CC1] | 306 | >10 | |

### Example 6: 2-(4-(2-fluoro-3-(methoxymethyl)phenyl)-3,6-dihydropyridin-1(2H)-yl)-N-isobutyl-3-methoxypropanamide (55)

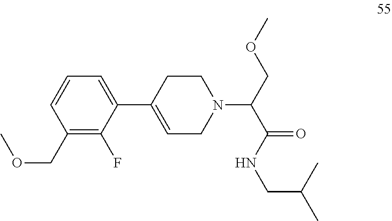 [SMILES: COCc1cccc(C2=CCN(C(COC)C(=O)NCC(C)C)CC2)c1F]

2-(4-(2-fluoro-3-(methoxymethyl)phenyl)-3,6-dihydropyridin-1(2H)-yl)-3-methoxypropanoic acid (Example 6a) (500 mg, 1.5 mmol), HOBt (229 mg, 1.5 mmol), EDC (286 mg, 1.5 mmol), triethylamine (151 mg, 1.5 mmol), and isobutyl amine (109 mg, 1.5 mmol) were suspended in DMF (10 mL) and the reaction was irradiated in the microwave at 110° C. for 30 minutes. The reaction mixture was diluted with ethyl acetate and washed with water. The organic layer was collected, dried over sodium sulfate, and evaporated under vacuum. The compound was purified by HPLC (Acetonitrile/Water) and concentrated under vacuum to give the desired product as a clear oil (176 mg, 31%). MS 379 (MH+).

### Example 6a: 2-(4-(2-fluoro-3-(methoxymethyl)phenyl)-3,6-dihydropyridin-1(2H)-yl)-3-methoxypropanoic acid

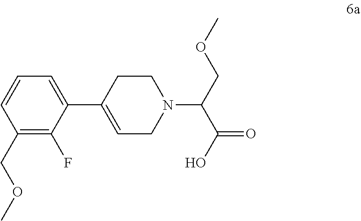 [SMILES: COCc1cccc(C2=CCN(C(COC)C(=O)O)CC2)c1F]

4-(2-fluoro-3-(methoxymethyl)phenyl)-1,2,3,6-tetrahydropyridine hydrogen chloride (Example 6b) (1 g, 3.4 mmol) and sodium hydride (266 mg, 11.6 mmol) were suspended in DMF (10 mL) and stirred at room temperature for 1 hour. Methyl 2-bromo-3-methoxypropanoate (1.3 g, 6.8 mmol) was added to the reaction mixture and the reaction was stirred overnight at room temperature, diluted with ethyl acetate, and was washed with water. The organic layer was collected, dried over sodium sulfate, and evaporated under vacuum to give a residue that was purified by column chromatography (EtOAc/Hexanes). The clean fractions were combined and concentrated under vacuum. The residue was redissolved in methanol (10 mL) and 1 M NaOH (5 mL) was added. The reaction was stirred overnight at room temperature, acidified to pH 6, concentrated under vacuum, and purified by HPLC to give the desired product (500 mg, 45%). MS 324 (M+H⁺).

### Example 6b: 4-((2-fluoro-3-(methoxymethyl)phenyl)-1,2,3,6-tetrahydropyridine hydrogen chloride

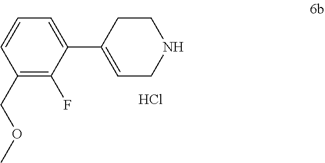 [SMILES: COCc1cccc(C2=CCNCC2)c1F ; Cl]

1-bromo-2-fluoro-3-(methoxymethyl)benzene (1.3 g, 6 mmol), tert-butyl 4-(4,4,5,5-tetramethyl-1,3,2-dioxaborolan-2-yl)-3,6-dihydropyridine-1(2H)-carboxylate (1.85 g, 6 mmol), potassium carbonate (2.4 g, 18 mmol) were suspended in 10 mL DME/water (5:1) followed by the addition of Pd(PPh₃)₄ (70 mg, 0.06 mmol). The reaction was irradiated in the microwave at 150° C. for 30 minutes, then diluted with water and extracted with ethyl acetate. The organic layer was dried over sodium sulfate, evaporated under vacuum, and purified by column chromatography (hexanes/ethyl acetate). The combined fractions were concentrated under vacuum and treated with 4M HCl in dioxane. The reaction was stirred for 30 minutes and solvent was evaporated under vacuum to give the desired hydrochloride salt as a light yellow oil. (1 g, 65%). MS 222 (M+H⁺).

Compounds in Table 2 were prepared in a similar manner as in Example 6 from the corresponding commercially available building blocks.

TABLE 2
Structures and data for selected compounds.

| | | Observed | Sweet | |
|---|---|---|---|---|
| | | Molecular Ion | EC₅₀ | |
| Cpd # | Structure | MS (M + 1) | (μM) | ¹H NMR (400 MHz) |
| 56 | 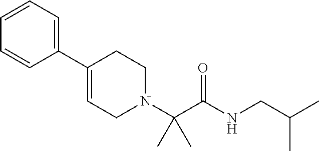 [SMILES: CC(C)CNC(=O)C(C)(C)N1CC=C(c2ccccc2)CC1] | 301 | 3.3 | |
| 57 | 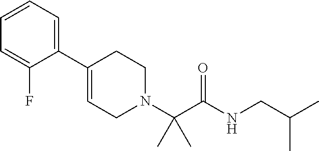 [SMILES: CC(C)CNC(=O)C(C)(C)N1CC=C(c2ccccc2F)CC1] | 319 | 1.6 | |
| 58 | 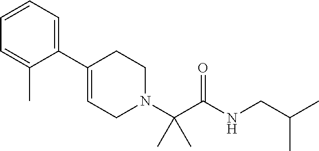 [SMILES: Cc1ccccc1C1=CCN(C(C)(C)C(=O)NCC(C)C)CC1] | 315 | 5.4 | |
| 59 | 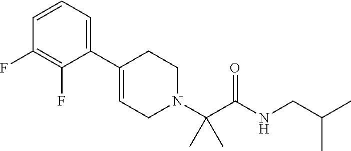 [SMILES: CC(C)CNC(=O)C(C)(C)N1CC=C(c2cccc(F)c2F)CC1] | 337 | 1.7 | |
| 60 | 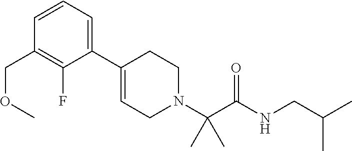 [SMILES: COCc1cccc(C2=CCN(C(C)(C)C(=O)NCC(C)C)CC2)c1F] | 363 | 1.2 | (DMSO-d₆ δ 0.80-0.84 (m, 6H), 0.89 (m, 2H), 1.16 (s, 6H), 1.64-1.85 (m, 3H), 2.92 (t, J = 6.5 Hz, 2H), 3.25 (d, J = 3.3 Hz, 2H), 3.30 (s, 3H), 4.45 (d, J = 1.3 Hz, 2H), 6.02 (s, 1H), 7.16 (t, J = 7.6 Hz, 1H), 7.30 (m, 2H), 7.75 (t, J = 6.2 Hz, 1H). |
| 55 | 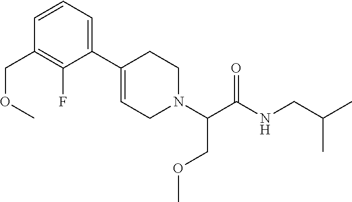 [SMILES: COCc1cccc(C2=CCN(C(COC)C(=O)NCC(C)C)CC2)c1F] | 379 | 1.4 | |
| 61 | 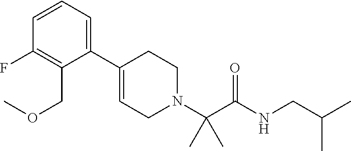 [SMILES: COCc1c(F)cccc1C1=CCN(C(C)(C)C(=O)NCC(C)C)CC1] | 363 | 6.5 | (MeOD) δ 0.94 (d, J = 6.4 Hz, 6H), 1.29 (s, 6H), 1.79-1.85 (m, 1H), 2.45 (m, 2H), 2.70 (m, 2H), 3.06 (d, J = 5.6 Hz, 2H), 3.26 (m, 2H), 3.37 (s, 3H), 4.47 (s, 2H), 5.73 (br.s, 1H), 6.99-7.04 (m, 2H), 7.32 (q, J = 7.1 Hz, 1H), 8.42 (s, 1H). |
| 62 | 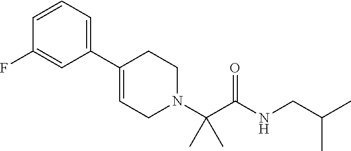 [SMILES: CC(C)CNC(=O)C(C)(C)N1CC=C(c2cccc(F)c2)CC1] | 319 | 3 | |
| 63 | 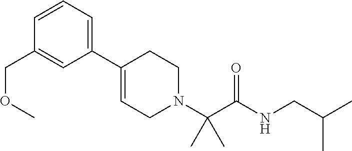 [SMILES: COCc1cccc(C2=CCN(C(C)(C)C(=O)NCC(C)C)CC2)c1] | 345 | >10 | (DMSO-d₆) δ 0.85 (d, J = 6.8 Hz, 6H), 1.15 (s, 6H), 1.71-1.78 (m, 1H), 2.33 (m, 2H), 2.52 (m, 2H), 2.94 (t, J = 6.4 Hz, 2H), 3.21 (m, 2H), 3.29 (s, 3H), 4.37 (s, 2H), 5.62 (s, 1H), 7.14-7.16 (m, 1H), 7.25-7.27 (m, 2H), 7.37-7.39 (m, 1H), 7.74 (t, J = 6.4 Hz, 1H). |
| 64 | 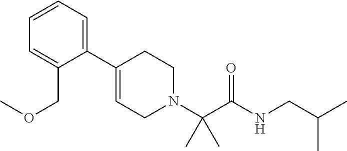 [SMILES: COCc1ccccc1C1=CCN(C(C)(C)C(=O)NCC(C)C)CC1] | 345 | >10 | |
| 65 | 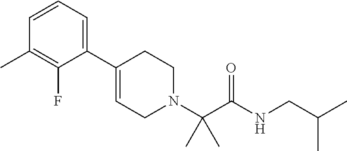 [SMILES: Cc1cccc(C2=CCN(C(C)(C)C(=O)NCC(C)C)CC2)c1F] | 333 | 2.9 | (DMSO-d₆) δ 0.84 (dd, J = 6.7, 4.2 Hz, 6H), 1.14 (s, 6H), 1.75 (m, 1H), 2.22 (d, J = 2.3 Hz, 3H), 2.47 (m, 4H), 2.92 (t, J = 6.5 Hz, 2H), 3.24 (t, J = 3.0 Hz, 2H), 5.99 (s, 1H), 7.05 (t, J = 7.6 Hz, 1H), 7.09-7.24 (m, 2H), 7.75 (t, J = 6.3 Hz, 1H). |
| 66 | 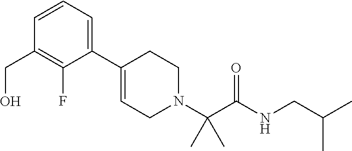 [SMILES: CC(C)CNC(=O)C(C)(C)N1CC=C(c2cccc(CO)c2F)CC1] | 349 | >10 | |
| 67 | 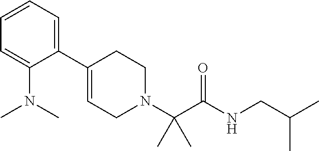 [SMILES: CC(C)CNC(=O)C(C)(C)N1CC=C(c2ccccc2N(C)C)CC1] | 344 | >10 | |
| 68 | 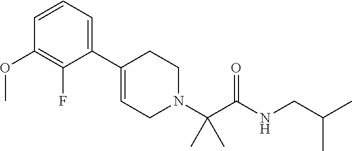 [SMILES: COc1cccc(C2=CCN(C(C)(C)C(=O)NCC(C)C)CC2)c1F] | 349 | >10 | |
| 69 | 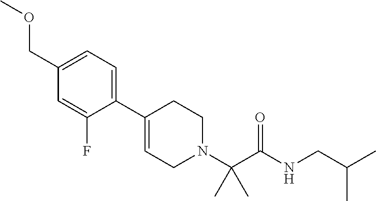 [SMILES: COCc1ccc(C2=CCN(C(C)(C)C(=O)NCC(C)C)CC2)c(F)c1] | 363 | >10 | |
| 70 | 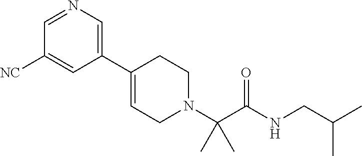 [SMILES: CC(C)CNC(=O)C(C)(C)N1CC=C(c2cncc(C#N)c2)CC1] | 327 | >10 | |
| 71 | 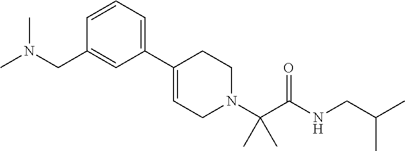 [SMILES: CC(C)CNC(=O)C(C)(C)N1CC=C(c2cccc(CN(C)C)c2)CC1] | 358 | >10 | |
| 72 | 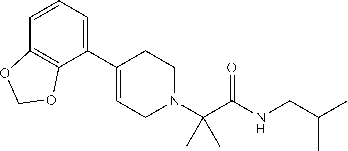 [SMILES: CC(C)CNC(=O)C(C)(C)N1CC=C(c2cccc3c2OCO3)CC1] | 345 | >10 | |
| 73 | 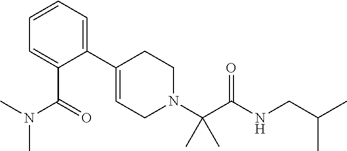 [SMILES: CC(C)CNC(=O)C(C)(C)N1CC=C(c2ccccc2C(=O)N(C)C)CC1] | 372 | >10 | |
| 74 | 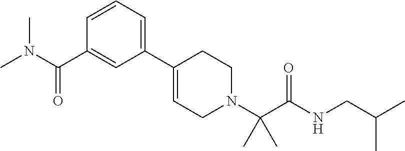 [SMILES: CC(C)CNC(=O)C(C)(C)N1CC=C(c2cccc(C(=O)N(C)C)c2)CC1] | 372 | >10 | |
| 75 | 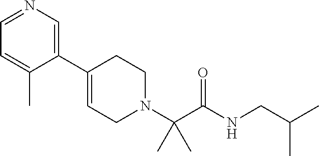 [SMILES: Cc1ccncc1C1=CCN(C(C)(C)C(=O)NCC(C)C)CC1] | 316 | >10 | |
| 76 | 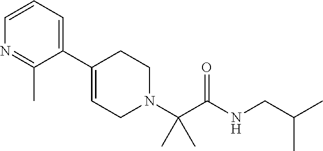 [SMILES: Cc1ncccc1C1=CCN(C(C)(C)C(=O)NCC(C)C)CC1] | 316 | >10 | |
| 77 | 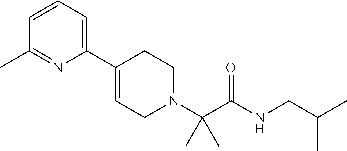 [SMILES: Cc1cccc(C2=CCN(C(C)(C)C(=O)NCC(C)C)CC2)n1] | 316 | >10 | |

### Example 7: 2-(4-(2-fluoro-3-(methoxymethyl)phenyl)piperidin-1-yl)-N-isobutyl-3-methoxypropanamide (78)

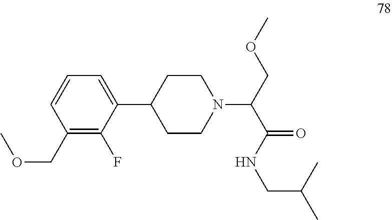 [SMILES: COCc1cccc(C2CCN(C(COC)C(=O)NCC(C)C)CC2)c1F]

2-(4-(2-fluoro-3-(methoxymethyl)phenyl)-3,6-dihydropyridin-1(2H)-yl)-N-isobutyl-3-methoxypropanamide (Example 6) (176 mg, 0.54 mmol) was suspended in methanol (20 mL) and 10% Pd/C (5 mol %) was added. The reaction was stirred overnight at room temperature under hydrogen balloon, filtered and concentrated under vacuum. The residue was purified by column chromatography (EtOAc/Hexanes) and concentrated under vacuum to give a residue that was suspended in water and lyophilized to afford the title compound as a white solid (135 mg, 65%). ¹H NMR (DMSO-d₆) δ 0.85 (d, J=6.7 Hz, 6H), 1.60-1.78 (m, 5H), 2.36 (td, J=11.1, 5.4 Hz, 1H), 2.75 (p, J=8.7, 8.1 Hz, 1H), 2.81-3.04 (m, 4H), 3.18-3.22 (m, 1H), 3.23 (d, J=0.4 Hz, 3H), 3.29 (s, 3H), 3.55 (dd, J=9.9, 6.1 Hz, 1H), 3.66 (dd, J=9.9, 6.0 Hz, 1H), 4.40-4.46 (m, 2H), 7.14 (t, J=7.6 Hz, 1H), 7.21-7.33 (m, 2H), 7.87 (t, J=6.1 Hz, 1H). MS 381 (M+H⁺).

### Example 8: 2-(4-cyano-4-(2,3-difluorophenyl)piperidin-1-yl)-N-isobutyl-2-methylpropanamide (79)

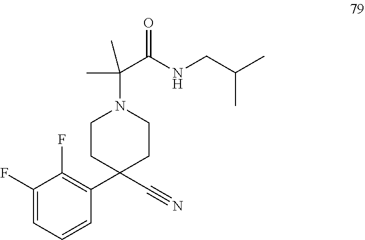 [SMILES: CC(C)CNC(=O)C(C)(C)N1CCC(C#N)(c2cccc(F)c2F)CC1]

To a solution of 4-(2,3-difluorophenyl)piperidine-4-carbonitrile hydrochloride (Example 8a) (0.4 g, 2 mmol) in anhydrous DMF (10 mL) was added sodium hydride (0.2 g, 4.4 mmol) and stirred at room temperature for 30 minutes, then 2-bromo-N-isobutyl-2-methylpropanamide (0.4 g, 2 mmol) was added and stirred overnight. The reaction was quenched with methanol (5 mL) and concentrated. The residue was purified by biotage using DCM/ethyl acetate gradient (0-30%). The clean fractions were combined and concentrated under vacuum to give a white solid, 230 mg (0.63 mmol), 32% yield. ¹H NMR (400 MHz, DMSO-d₆) δ 0.81 (d, J=6.7 Hz, 6H), 1.13 (s, 6H), 1.67-1.77 (m, 1H), 2.17 (td, J=12.2, 3.4 Hz, 2H), 2.28 (d, J=13.7 Hz, 2H), 2.46 (d, J=2.1 Hz, 1H), 2.52 (d, J=2.0 Hz, 1H), 2.81-2.92 (m, 4H), 7.27-7.38 (m, 2H), 7.49-7.60 (m, 1H), 7.79 (t, J=6.2 Hz, 1H). MS 364 (M+H⁺).

### Example 8a: 4-(2,3-difluorophenyl)piperidine-4-carbonitrile hydrochloride

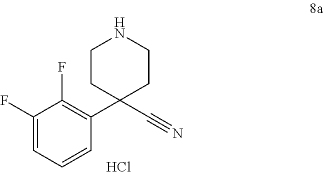 [SMILES: N#CC1(c2cccc(F)c2F)CCNCC1 ; Cl]

To a solution of tert-butyl 4-cyano-4-(2,3-difluorophenyl)piperidine-1-carboxylate (Example 8b) (1.4 g, 4.2 mmol) dissolved in dioxane (10 mL) was added 4 M HCl in dioxane (10 mL) and stirred at room temperature for 2 hours. The solid precipitate was filtered, washed with dioxane, and dried under vacuum to give the desired material as a white solid (0.8 g, 3 mmol, 74% yield). MS 223.

### Example 8b: tert-butyl 4-cyano-4-(2,3-difluorophenyl)piperidine-1-carboxylate

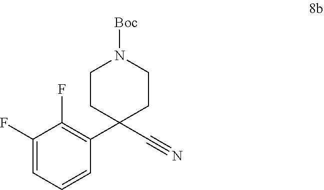 [SMILES: CC(C)(C)OC(=O)N1CCC(C#N)(c2cccc(F)c2F)CC1]

To a solution of tert-butyl bis(2-chloroethyl)carbamate (5.3 g, 22 mmol) and 2-(2,3-difluorophenyl)acetonitrile (3 g, 20 mmol) in anhydrous DMF (50 mL) was cooled to 0° C. in an ice bath and sodium hydride (2.3 g, 60 mmol) was added portion wise with bubbler attached. The reaction was heated to 60° C. for 1.5 hours; then stirred at room temperature overnight. The reaction was quenched by the addition of saturated ammonium chloride (aq) (70 mL) and then extracted with DCM (3×50 mL). The organic layer was washed with brine, dried over MgSO₄ and concentrated to give 5 g (15.5 mmol), 78% yield of the crude product which was used without further purification. MS 323 (M+H⁺).

### Example 9: N-isobutyl-2-methyl-2-(4-phenylpiperidin-1-yl)propanamide (80)

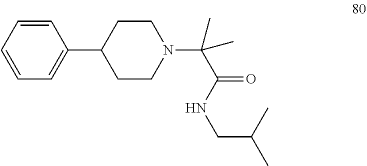 [SMILES: CC(C)CNC(=O)C(C)(C)N1CCC(c2ccccc2)CC1]

To a solution of 4-phenylpiperidine (800 mg, 4.96 mmol) in dry THF (60 mL), was added sodium hydride (1 eq.), the mixture was stirred at room temperature for about 45 min., 2-bromo-N-isobutyl-2-methylpropanamide (1.32 g, 5.95 mmol) was added, the reaction was heated to 75° C. and stirred under nitrogen overnight. The resulting mixture was concentrated under reduced pressure, diluted with EtOAc, washed with water and brine, the organic layer was dried over MgSO₄, filtered, concentrated under reduced pressure and the residue was purified by silica gel chromatography (EtOAc/hexane 1:4). Clean fractions were concentrated under reduced pressure to afford 1.18 g (yield 78%) of the title compound as a white solid. MS 303 (M+H⁺).

### Example 10: 4-Hydroxy-N-isobutyl-2-(4-phenylpiperidin-1-yl)butanamide (81)

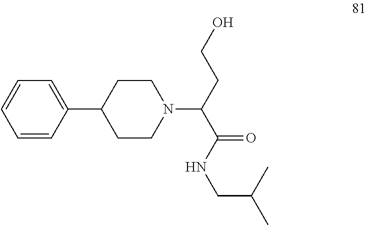 [SMILES: CC(C)CNC(=O)C(CCO)N1CCC(c2ccccc2)CC1]

A mixture of 3-bromodihydrofuran-2(3H)-one (200 mg, 1.21 mmol) and 4-phenylpiperidine (200 mg, 1.21 mmol) in acetonitrile (6 mL) was stirred at room temperature for 10 min., and then TEA (2 eq.) was added. The reaction mixture was heated at 45° C. overnight and then 2-methylpropan-1-amine (10 eq.) was added. The reaction was heated to 85° C. and stirred for 24 hours, then cooled to room temperature, concentrated under reduced pressure, and purified by Mass triggered HPLC to obtain 320 mg (yield 83%) of title compound as a yellow gel. MS 319 (M+H⁺).

Compounds in Table 3 were prepared in a similar manner as in Example 7, Example 8, Example 9, and/or Example 10 from the corresponding commercially available building blocks.

TABLE 3
Structures and data for selected compounds.

| | | Observed | Sweet | |
|---|---|---|---|---|
| | | Molecular Ion | EC₅₀ | |
| Cpd # | Structure | MS (M + 1) | (μM) | ¹H NMR (400 MHz) |
| 82 | 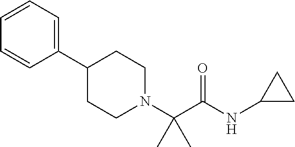 [SMILES: CC(C)(C(=O)NC1CC1)N1CCC(c2ccccc2)CC1] | 287 | 10.4 | (DMSO-d₆) δ 0.44-0.48 (m, 2H), 0.58- 0.64 (m, 2H), 1.07 (s, 6H), 1.63- 1.78 (m, 4H), 2.16 (td, J = 11.2, 3.2 Hz, 2H), 2.40-2.47 (m, 1H), 2.58-2.66 (m, 1H), 2.74 (d, J = 11.2 Hz, 2H), 7.15-7.20 (m, 1H), 7.23-7.31 (m, 4H), 7.58 (d, J = 4.5 Hz, 1H). |
| 83 | 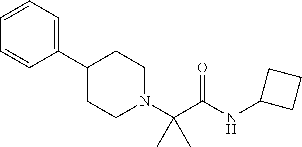 [SMILES: CC(C)(C(=O)NC1CCC1)N1CCC(c2ccccc2)CC1] | 301 | >10 | (DMSO-d₆) δ 1.07 (s, 6H), 1.54-1.67 (m, 2H), 1.67-1.80 (m, 4H), 1.90- 2.03 (m, 2H), 2.08-2.22 (m, 4H), 2.42- 2.50 (m, 1H), 2.76 (d, J = 11.1 Hz, 2H), 4.16-4.29 (m, 1H), 7.16-7.22 (m, 1H), 7.26-7.33 (m, 4H), 7.76 (d, J = 8.7 Hz, 1H). |
| 84 | 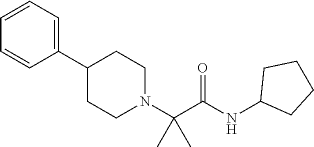 [SMILES: CC(C)(C(=O)NC1CCCC1)N1CCC(c2ccccc2)CC1] | 315 | 2.5 | (DMSO-d₆) δ 1.08 (s, 6H), 1.37-1.46 (m, 2H), 1.48-1.58 (m, 2H), 1.59- 1.71 (m, 4H), 1.71-1.83 (m, 5H), 2.12- 2.23 (m, 2H), 2.43-2.49 (m, 1H), 2.77 (d, J = 11.5 Hz, 2H), 4.01 (h, J = 6.7 Hz, 1H), 7.15-7.21 (m, 1H), 7.23- 7.32 (m, 4H), 7.44 (d, J = 8.1 Hz, 1H). |
| 85 | 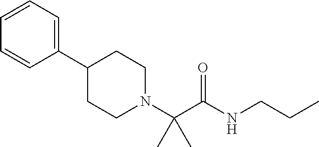 [SMILES: CCCNC(=O)C(C)(C)N1CCC(c2ccccc2)CC1] | 289 | 5.5 | (DMSO-d₆) δ 0.83 (t, J = 7.4 Hz, 3H), 1.09 (s, 6H), 1.42 (h, J = 7.3 Hz, 2H), 1.67-1.78 (m, 4H), 2.14-2.23 (m, 2H), 2.43-2.50 (m, 1H), 2.80 (d, J = 11.2 Hz, 2H), 3.05 (q, J = 6.7 Hz, 2H), 7.15-7.21 (m, 1H), 7.23-7.32 (m, 4H), 7.74 (t, J = 6.1 Hz, 1H). |
| 86 | 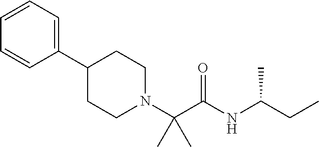 [SMILES: CC[C@@H](C)NC(=O)C(C)(C)N1CCC(c2ccccc2)CC1] | 317 | 4.4 | (DMSO-d₆) δ 0.84 (d, J = 6.8 Hz, 6H), 1.00 (d, J = 6.7 Hz, 3H), 1.10 (d, J = 4.8 Hz, 6H), 1.61-1.75 (m, 3H), 1.79 (d, J = 12.6 Hz, 2H), 2.14-2.26 (m, 2H), 2.43-2.55 (m, 1H), 2.81 (t, J = 11.2 Hz, 2H), 3.54-3.65 (m, 1H), 7.15- 7.21 (m, 1H), 7.22-7.33 (m, 4H), 7.36 (d, J = 9.3 Hz, 1H). |
| 87 | 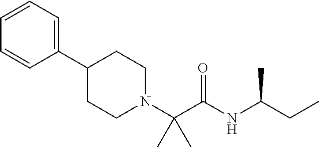 [SMILES: CC[C@H](C)NC(=O)C(C)(C)N1CCC(c2ccccc2)CC1] | 317 | 4.8 | (DMSO-d₆) δ 0.84 (d, J = 6.8 Hz, 6H), 1.00 (d, J = 6.7 Hz, 3H), 1.10 (d, J = 4.8 Hz, 6H), 1.60-1.74 (m, 3H), 1.79 (d, J = 11.6 Hz, 2H), 2.07-2.27 (m, 2H), 2.43-2.55 (m, 1H), 2.81 (t, J = 11.2 Hz, 2H), 3.54-3.65 (m, 1H), 7.15- 7.22 (m, 1H), 7.22-7.33 (m, 4H), 7.36 (d, J = 9.3 Hz, 1H). |
| 88 | 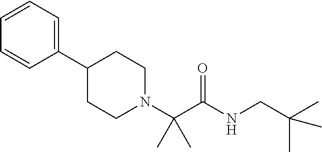 [SMILES: CC(C)(C)CNC(=O)C(C)(C)N1CCC(c2ccccc2)CC1] | 317 | 4.2 | (DMSO-d₆) δ 0.85 (s, 9H), 1.11 (s, 6H), 1.69 (qd, J = 12.1, 3.6 Hz, 2H), 1.81 (d, J= 11.7 Hz, 2H), 2.23 (td, J = 11.6, 2.2 Hz, 2H), 2.47-2.55 (m, 1H), 2.85 (d, J = 11.2 Hz, 2H), 2.93 (d, J = 6.6 Hz, 2H), 7.16-7.21 (m, 1H), 7.22-7.27 (m, 2H), 7.27-7.33 (m, 2H), 7.65 (t, J = 6.6 Hz, 1H). |
| 89 | 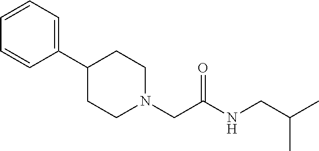 [SMILES: CC(C)CNC(=O)CN1CCC(c2ccccc2)CC1] | 275 | 6.7 | (DMSO-d₆) δ 0.84 (d, J = 6.7 Hz, 6H), 1.64-1.81 (m, 5H), 2.10-2.23 (m, 2H), 2.43-2.49 (m, 1H), 2.86-2.96 (m, 6H), 7.14-7.21 (m, 1H), 7.22- 7.33 (m, 4H), 7.74 (t, J = 6.0 Hz, 1H). |
| 90 | 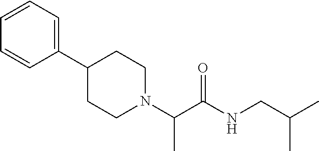 [SMILES: CC(C)CNC(=O)C(C)N1CCC(c2ccccc2)CC1] | 289 | 4.7 | (DMSO-d₆) δ 0.84 (d, J = 6.7 Hz, 6H), 1.10 (d, J = 6.9 Hz, 3H), 1.63-1.79 (m, 5H), 2.18 (td, J = 11.3, 2.8 Hz, 1H), 2.34 (td, J = 11.2, 3.1 Hz, 1H), 2.41- 2.48 (m, 1H), 2.79-2.91 (m, 3H), 2.94 3.03 (m, 1H), 3.06 (q, J = 6.9 Hz, 1H), 7.15-7.21 (m, 1H), 7.22-7.32 (m, 4H), 7.78 (t, J = 6.1 Hz, 1H). |
| 80 | 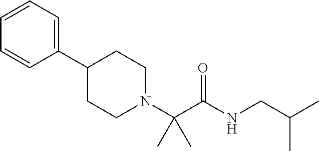 [SMILES: CC(C)CNC(=O)C(C)(C)N1CCC(c2ccccc2)CC1] | 302 | 0.7 | (DMSO-d₆) δ 0.83 (d, J = 6.7 Hz, 6H), 1.10 (s, 6H), 1.74 (m, 5H), 2.20 (td, J = 11.1, 3.3 Hz, 2H), 2.81 (d, J = 11.1 Hz, 2H), 2.92 (t, J = 6.5 Hz, 2H), 7.14- 7.21 (m, 1H), 7.23-7.33 (m, 4H), 7.75 (t, J = 6.2 Hz, 1H). |
| 91 | 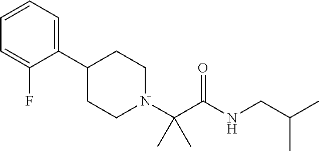 [SMILES: CC(C)CNC(=O)C(C)(C)N1CCC(c2ccccc2F)CC1] | 321 | 0.4 | (DMSO-d₆) δ 0.83 (d, J = 6.7 Hz, 6H), 1.10 (s, 6H), 1.78 (m, 6H), 2.22 (m, 2H), 2.81 (m, 2H), 2.92 (t, J = 6.5 Hz, 2H), 7.08-7.19 (m, 2H), 7.25 (m, 1H), 7.36 (m, 1H), 7.74 (t, J = 6.1 Hz, 1H). |
| 92 | 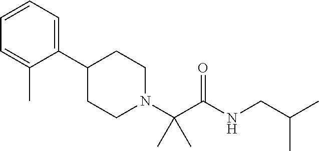 [SMILES: Cc1ccccc1C1CCN(C(C)(C)C(=O)NCC(C)C)CC1] | 317 | 1.6 | (DMSO-d₆) δ 0.84 (d, J = 6.7 Hz, 6H), 1.10 (s, 6H), 1.63-1.81 (m, 5H), 2.23 (m, 2H), 2.28 (s, 3H), 2.70 (m, 1H), 2.82 (d, J = 11.1 Hz, 2H), 2.93 (t, J = 6.5 Hz, 2H), 7.03-7.19 (m, 3H), 7.22- 7.28 (m, 1H), 7.76 (t, J = 6.2 Hz, 1H). |
| 93 | 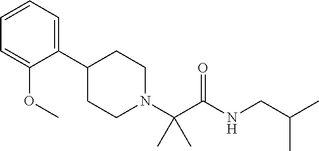 [SMILES: COc1ccccc1C1CCN(C(C)(C)C(=O)NCC(C)C)CC1] | 330 | 2.8 | (DMSO-d6) 8 0.83 (d, J = 6.7 Hz, 6H), 1.09 (s, 6H), 1.61-1.81 (m, 5H), 2.20 (td, J = 11.3, 10.7, 4.3 Hz, 2H), 2.80 (d, J = 11.2 Hz, 2H), 2.83-2.89 (m, 1H), 2.92 (t, J = 6.5 Hz, 2H), 3.77 (s, 3H), 6.88-6.97 (m, 2H), 7.13-7.23 (m, 2H), 7.73 (t, J = 6.2 Hz, 1H). |
| 94 | 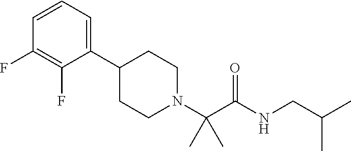 [SMILES: CC(C)CNC(=O)C(C)(C)N1CCC(c2cccc(F)c2F)CC1] | 339 | 0.14 | δ 0.78-0.87 (d, J = 8, 6H), 1.10 (s, 6H), 1.62-1.84 (m, 5H), 2.22 (m, 2H), 2.82 (d, J = 10.9 Hz, 3H), 2.92 (t, J = 6.5 Hz, 2H), 7.16-7.21 (m, 2H), 7.21- 7.33 (m, 1H), 7.74 (t, J = 5.9 Hz, 1H). |
| 95 | 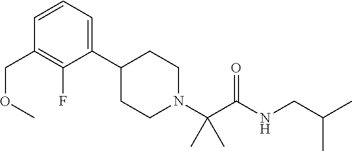 [SMILES: COCc1cccc(C2CCN(C(C)(C)C(=O)NCC(C)C)CC2)c1F] | 365 | 0.5 | (DMSO-d₆) δ 0.83 (d, J = 6.7 Hz, 6H), 1.10 (s, 6H), 1.75 (m, 5H), 2.22 (m, 2H), 2.76-2.86 (m, 3H), 2.92 (t, J = 6.5 Hz, 2H), 3.29 (s, 3H), 4.44 (d, J = 1.2 Hz, 2H), 7.16 (t, J = 7.6 Hz, 1H), 7.28 (m, 2H), 7.74 (t, J = 6.2 Hz, 1H). |
| 96 | 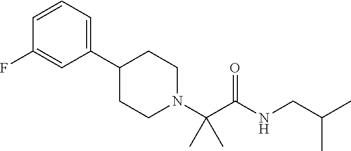 [SMILES: CC(C)CNC(=O)C(C)(C)N1CCC(c2cccc(F)c2)CC1] | 321 | 0.6 | (DMSO-d₆) δ 0.83 (d, J = 6.7 Hz, 6H), 1.09 (s, 6H), 1.73 (m, 5H), 2.12-2.25 (m, 3H), 2.81 (d, J = 11.1 Hz, 2H), 2.92 (t, J = 6.5 Hz, 2H), 6.95-7.20 (m, 3H), 7.34 (m, 1H), 7.75 (t, J = 6.5 Hz, 1H). |
| 97 | 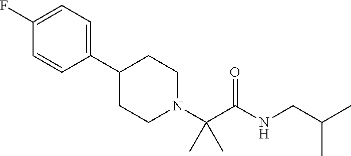 [SMILES: CC(C)CNC(=O)C(C)(C)N1CCC(c2ccc(F)cc2)CC1] | 321 | 6.2 | (DMSO-d₆) δ 0.83 (d, J = 6.7 Hz, 6H), 1.09 (s, 6H), 1.62-1.80 (m, 5H), 2.19 (td, J = 11.3, 2.8 Hz, 2H), 2.46-2.55 (m, 1H), 2.80 (d, J = 11.3 Hz, 2H), 2.92 (t, J = 6.5 Hz, 2H), 7.08-7.15 (m, 2H), 7.26-7.32 (m, 2H), 7.74 (t, J = 6.2 Hz, 1H). |
| 98 | 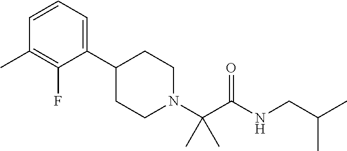 [SMILES: Cc1cccc(C2CCN(C(C)(C)C(=O)NCC(C)C)CC2)c1F] | 335 | 0.9 | (DMSO-d₆) δ 0.83 (d, J = 6.7 Hz, 6H), 1.10 (s, 6H), 1.66-1.81 (m, 6H), 2.21 (m, 3H), 2.71-2.86 (m, 4H), 2.92 (t, J = 6.5 Hz, 2H), 7.05 (t, J = 7.5 Hz, 1H), 7.08-7.19 (m, 2H), 7.73 (t, J = 6.2 Hz, 1H). |
| 99 | 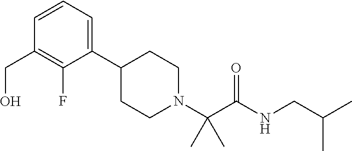 [SMILES: CC(C)CNC(=O)C(C)(C)N1CCC(c2cccc(CO)c2F)CC1] | 351 | 4.7 | (DMSO-d₆) δ 0.82-0.86 (m, 6H), 1.10 (s, 6H), 1.65-1.84 (m, 5H), 2.14- 2.29 (m, 2H), 2.74-2.82 (m, 2H), 2.92 (t, J = 6.5 Hz, 2H), 4.52 (d, J = 5.4 Hz, 2H), 5.21 (t, J = 5.7 Hz, 1H), 7.14 (t, J = 7.6 Hz, 1H), 7.27 (m, 2H), 7.74 (t, J = 6.2 Hz, 1H). |
| 100 | 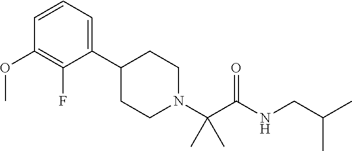 [SMILES: COc1cccc(C2CCN(C(C)(C)C(=O)NCC(C)C)CC2)c1F] | 351 | 1.4 | (DMSO-d₆) δ 0.83 (d, J = 6.7 Hz, 6H), 1.09 (s, 6H), 1.66-1.80 (m, 5H), 2.15- 2.26 (m, 2H), 2.81 (m, 2H), 2.92 (t, J = 6.5 Hz, 2H), 3.80 (s, 3H), 6.87-6.91 (m, 1H), 6.99 (m, 1H), 7.09 (m, 1H), 7.73 (t, J = 6.2 Hz, 1H). |
| 101 | 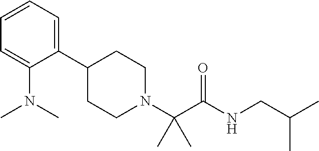 [SMILES: CC(C)CNC(=O)C(C)(C)N1CCC(c2ccccc2N(C)C)CC1] | 346 | >10 | |
| 102 | 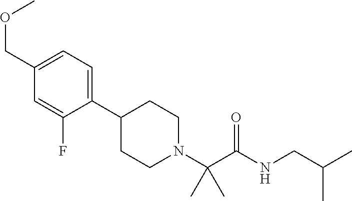 [SMILES: COCc1ccc(C2CCN(C(C)(C)C(=O)NCC(C)C)CC2)c(F)c1] | 365 | >10 | |
| 103 | 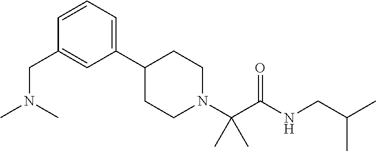 [SMILES: CC(C)CNC(=O)C(C)(C)N1CCC(c2cccc(CN(C)C)c2)CC1] | 360 | >10 | |
| 104 | 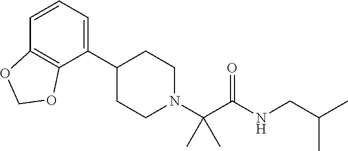 [SMILES: CC(C)CNC(=O)C(C)(C)N1CCC(c2cccc3c2OCO3)CC1] | 347 | 1.7 | (DMSO-d₆) δ 0.83 (d, J = 6.7 Hz, 6H), 1.09 (s, 6H), 1.64-1.88 (m, 5H), 2.18 (m, 2H), 2.60 (m, 1H), 2.80 (d, J = 11.2 Hz, 2H), 2.92 (t, J = 6.5 Hz, 2H), 5.97 (s, 2H), 6.73-6.84 (m, 3H), 7.70 (t, J = 6.2 Hz, 1H). |
| 105 | 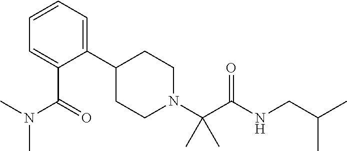 [SMILES: CC(C)CNC(=O)C(C)(C)N1CCC(c2ccccc2C(=O)N(C)C)CC1] | 374 | >10 | |
| 106 | 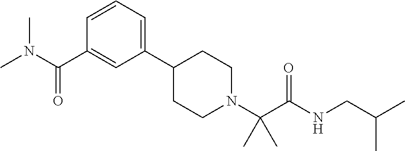 [SMILES: CC(C)CNC(=O)C(C)(C)N1CCC(c2cccc(C(=O)N(C)C)c2)CC1] | 374 | >10 | |
| 107 | 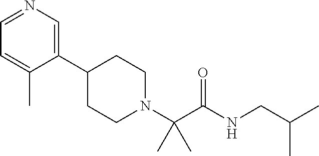 [SMILES: Cc1ccncc1C1CCN(C(C)(C)C(=O)NCC(C)C)CC1] | 318 | >10 | |
| 108 | 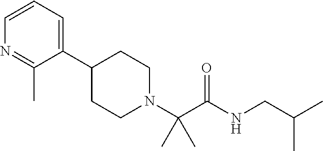 [SMILES: Cc1ncccc1C1CCN(C(C)(C)C(=O)NCC(C)C)CC1] | 318 | >10 | |
| 109 | 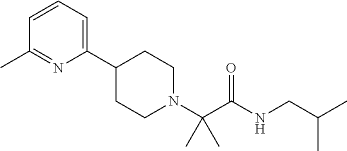 [SMILES: Cc1cccc(C2CCN(C(C)(C)C(=O)NCC(C)C)CC2)n1] | 318 | 5.1 | |
| 110 | 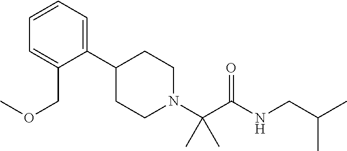 [SMILES: COCc1ccccc1C1CCN(C(C)(C)C(=O)NCC(C)C)CC1] | 347 | >10 | (MeOD) δ 0.92 (dd, J = 6.7, 0.9 Hz, 6H), 1.41 (s, 6H), 1.81-1.99 (m, 5H), 2.75 (m, 2H), 2.98-3.07 (m, 4H), 3.16- 3.19 (m, 2H), 3.35 (d, J = 0.8 Hz, 3H), 4.50 (s, 2H), 7.17 (t, J = 7.2 Hz, 1H), 7.27-7.36 (m, 3H). |
| 111 | 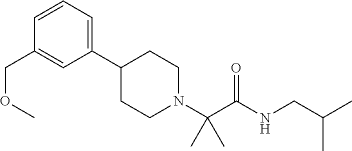 [SMILES: COCc1cccc(C2CCN(C(C)(C)C(=O)NCC(C)C)CC2)c1] | 347 | 3.1 | δ 0.83 (d, J = 6.7 Hz, 6H), 1.10 (s, 6H), 1.75 (m, 5H), 2.11-2.24 (m, 2H), 2.81 m, 2H), 2.92 (t, J = 6.5 Hz, 2H), 3.28 (s, 3H), 4.38 (s, 2H), 7.08-7.22 (m, 3H), 7.28 (m, 2H), 7.76 (t, J = 6.2 Hz, 1H). |
| 112 | 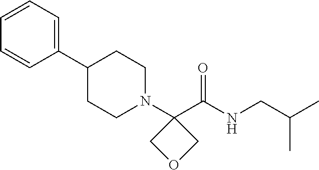 [SMILES: CC(C)CNC(=O)C1(N2CCC(c3ccccc3)CC2)COC1] | 317 | 4.8 | (DMSO-d₆) δ 0.88 (d, J = 6.7 Hz, 6H), 1.61-1.81 (m, 5H), 2.14-2.23 (m, 2H), 2.44-2.48 (m, 1H), 2.88 (d, J = 10.9 Hz, 2H), 2.98 (t, J = 6.3 Hz, 2H), 4.51 (d, J = 6.6 Hz, 2H), 4.60 (d, J = 6.5 Hz, 2H), 7.14-7.21 (m, 1H), 7.21- 7.32 (m, 4H), 7.79-7.86 (m, 1H). |
| 113 | 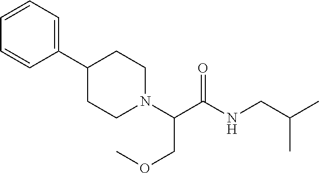 [SMILES: COCC(C(=O)NCC(C)C)N1CCC(c2ccccc2)CC1] | 319 | 4.4 | (DMSO-d₆) δ 1.55-1.64 (m, 2H), 1.69- 1.73 (m, 2H), 2.32 (td, J = 11.5, 2.2 Hz, 1H), 2.41-2.47 (m, 2H), 2.91 (dd, J = 27.4, 11.4 Hz, 2H), 3.25 (s, 3H), 3.45 (dd, J = 7.9, 5.3 Hz, 1H), 3.55 (dd, J = 9.4, 5.3 Hz, 1H), 3.65 (s, 3H), 3.67-3.70 (m, 1H), 7.17-7.30 (m, 5H). |
| 78 | 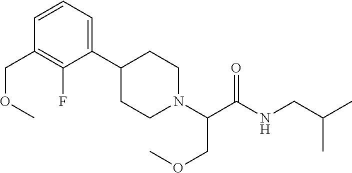 [SMILES: COCc1cccc(C2CCN(C(COC)C(=O)NCC(C)C)CC2)c1F] | 381 | 1.3 | (DMSO-d₆) δ 0.85 (d, J = 6.7 Hz, 6H), 1.60-1.78 (m, 5H), 2.36 (td, J = 11.1, 5.4 Hz, 1H), 2.75 (p, J = 8.7, 8.1 Hz, 1H), 2.81-3.04 (m, 4H), 3.18-3.22 (m, 1H), 3.23 (d, J = 0.4 Hz, 3H), 3.29 (s, 3H), 3.55 (dd, J = 9.9, 6.1 Hz, 1H), 3.66 (dd, J = 9.9, 6.0 Hz, 1H), 4.40- 4.46 (m, 2H), 7.14 (t, J = 7.6 Hz, 1H), 7.21-7.33 (m, 2H), 7.87 (t, J = 6.1 Hz, 1H). |
| 114 | 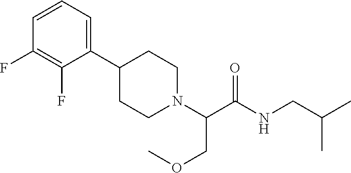 [SMILES: COCC(C(=O)NCC(C)C)N1CCC(c2cccc(F)c2F)CC1] | 355 | 0.4 | (DMSO-d₆) δ 0.85 (d, J = 6.6 Hz, 6H), 1.60-1.82 (m, 5H), 2.30-2.41 (m, 2H), 2.74-3.03 (m, 5H), 3.23 (s, 3H), 3.54 (dd, J = 9.9, 6.1 Hz, 1H), 3.66 (dd, J = 9.9, 6.0 Hz, 1H), 7.16 (dd, J = 6.6, 3.1 Hz, 2H), 7.20-7.30 (m, 1H), 7.88 (t, J = 6.1 Hz, 1H). |
| 115 | 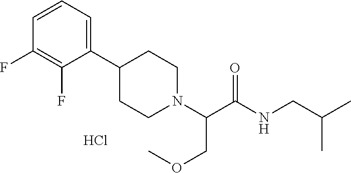 [SMILES: COCC(C(=O)NCC(C)C)N1CCC(c2cccc(F)c2F)CC1 ; Cl] | 355 | | ¹H NMR (DMSO-d₆, 400 MHz) δ: 0.81 (d, J = 6.4 Hz, 6H), 1.69 (m, 5H), 2.33 (m, 1H), 2.73-2.97 (m, 6H), 3.17 (m, 1H), 3.21 (s, 3H), 3.52 (m, 1H), 3.63 (m, 1H), 7.14-7.24 (m, 3H), 7.85 (t, J = 5.6 Hz, 1H). |
| 116 | 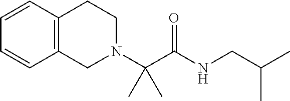 [SMILES: CC(C)CNC(=O)C(C)(C)N1CCc2ccccc2C1] | 275 | 3.8 | (DMSO-d₆) δ 0.81 (d, J = 6.7 Hz, 6H), 1.18 (s, 6H), 1.67-1.79 (m, 1H), 2.55 (t, J = 5.7 Hz, 2H), 2.83 (t, J = 5.7 Hz, 2H), 2.90 (t, J = 6.5 Hz, 2H), 3.71 (s, 2H), 7.06-7.13 (m, 4H), 7.78 (t, J = 6.2 Hz, 1H). |
| 117 | 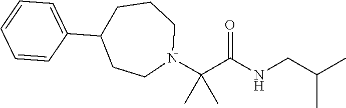 [SMILES: CC(C)CNC(=O)C(C)(C)N1CCCC(c2ccccc2)CC1] | 317 | 10.9 | (DMSO-d₆) δ 0.84 (dd, J = 6.7, 1.1 Hz, 6H), 1.11 (d, J = 7.2 Hz, 6H), 1.55- 1.68 (m, 1H), 1.68-1.92 (m, 6H), 2.53- 2.65 (m, 4H), 2.77-2.87 (m, 1H), 2.94 (t, J = 6.5 Hz, 2H), 7.11-7.17 (m, 1H), 7.20-7.31 (m, 4H), 7.59 (t, J = 6.3 Hz, 1H). |
| 118 | 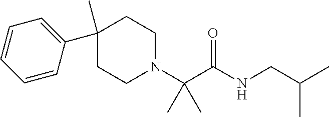 [SMILES: CC(C)CNC(=O)C(C)(C)N1CCC(C)(c2ccccc2)CC1] | 317 | 3.7 | (DMSO-d₆) δ 0.81 (dd, J = 6.8, 2.2 Hz, 6H), 1.04 (d, J = 2.1 Hz, 6H), 1.15 (d, J = 2.2 Hz, 3H), 1.64-1.82 (m, 3H), 2.06 (t, J = 9.7 Hz, 2H), 2.31-2.41 (m, 2H), 2.41-2.48 (m, 2H), 2.89 (td, J = 6.5, 2.2 Hz, 2H), 7.12-7.20 (m, 1H), 7.28-7.34 (m, 2H), 7.34-7.40 (m, 2H), 7.72 (t, J = 5.6 Hz, 1H). |
| 119 | 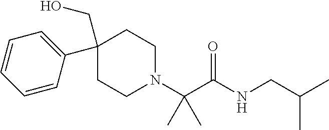 [SMILES: CC(C)CNC(=O)C(C)(C)N1CCC(CO)(c2ccccc2)CC1] | 333 | >10 | |
| 120 | 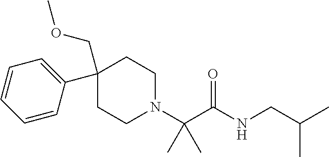 [SMILES: COCC1(c2ccccc2)CCN(C(C)(C)C(=O)NCC(C)C)CC1] | 347 | >10 | |
| 121 | 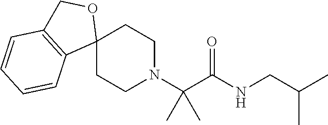 [SMILES: CC(C)CNC(=O)C(C)(C)N1CCC2(CC1)OCc1ccccc12] | 331 | 5.6 | (DMSO-d₆) δ 0.84 (d, J = 6.7 Hz, 6H), 1.11 (s, 6H), 1.64 (d, J = 11.2 Hz, 2H), 1.75 (hept, J = 6.7 Hz, 1H), 1.98 (td, J = 12.7, 4.4 Hz, 2H), 2.42-2.49 (m, 2H), 2.63 (d, J = 11.1 Hz, 2H), 2.93 (t, J = 6.5 Hz, 2H), 4.96 (s, 2H), 7.21- 7.32 (m, 4H), 7.82 (t, J = 6.3 Hz, 1H). |
| 122 | 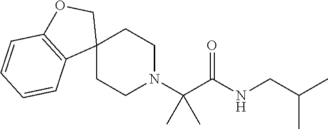 [SMILES: CC(C)CNC(=O)C(C)(C)N1CCC2(CC1)COc1ccccc12] | 331 | 2.5 | (DMSO-d₆) δ 0.84 (d, J = 6.7 Hz, 6H), 1.10 (s, 6H), 1.65 (d, J = 12.2 Hz, 2H), 1.75 (hept, J = 6.7 Hz, 1H), 1.94 (td, J = 12.5, 3.8 Hz, 2H), 2.11-2.24 (m, 2H), 2.70 (d, J = 11.7 Hz, 2H), 2.93 (t, J = 6.5 Hz, 2H), 4.34 (s, 2H), 6.75 (dt, J = 7.9, 0.7 Hz, 1H), 6.87 (td, J = 7.4, 1.0 Hz, 1H), 7.07-7.15 (m, 1H), 7.21 |
| | | | | (dd, J = 7.4, 1.3 Hz, 1H), 7.80 (t, J = |
| | | | | 6.2 Hz,1H). |
| 123 | 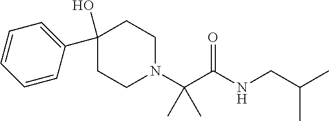 [SMILES: CC(C)CNC(=O)C(C)(C)N1CCC(O)(c2ccccc2)CC1] | 319 | >10 | (DMSO-d₆) δ 0.83 (d, J = 6.7 Hz, 6H), 1.11 (s, 6H), 1.59 (d, J = 11.8 Hz, 2H), 1.74 (hept, J = 6.8 Hz, 1H), 2.02 (td, J = 12.4, 4.7 Hz, 2H), 2.50-2.61 (m, 4H), 2.92 (t, J = 6.5 Hz, 2H), 4.78 (br-s, 1H), 7.17-7.24 (m, 1H), 7.29-7.35 (m, 2H), 7.49-7.55 (m, 2H), 7.85 (t, J = 6.2 Hz, 1H). |
| 124 | 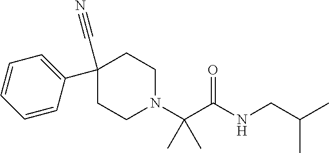 [SMILES: CC(C)CNC(=O)C(C)(C)N1CCC(C#N)(c2ccccc2)CC1] | 328 | 2.2 | (DMSO-d₆) δ 0.82 (d, J = 6.6 Hz, 6H), 1.13 (s, 6H), 1.74 (hept, J = 6.7 Hz, 1H), 2.04-2.24 (m, 4H), 2.44 (dd, J = 12.0, 2.6 Hz, 2H), 2.85 (d, J = 12.2 Hz, 2H), 2.91 (t, J = 6.5 Hz, 2H), 7.35- 7.41 (m, 1H), 7.43-7.49 (m, 2H), 7.54- 7.60 (m, 2H), 7.85 (t, J = 6.3 Hz, 1H). |
| 79 | 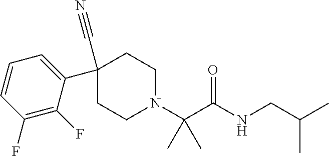 [SMILES: CC(C)CNC(=O)C(C)(C)N1CCC(C#N)(c2cccc(F)c2F)CC1] | 364 | 1.6 | (DMSO-d₆) δ 0.81 (d, J = 6.7 Hz, 6H), 1.13 (s, 6H), 1.67-1.77 (m, 1H), 2.17 (td, J = 12.2, 3.4 Hz, 2H), 2.28 (d, J = 13.7 Hz, 2H), 2.46 (d, J = 2.1 Hz, 1H), 2.52 (d, J = 2.0 Hz, 1H), 2.81-2.92 (m, 4H), 7.27-7.38 (m, 2H), 7.49 7.60 (m, 1H), 7.79 (t, J = 6.2 Hz, 1H). |
| 125 | 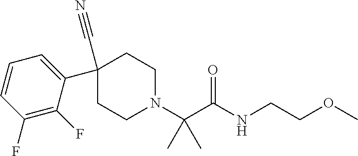 [SMILES: COCCNC(=O)C(C)(C)N1CCC(C#N)(c2cccc(F)c2F)CC1] | 366 | >10 | (DMSO-d₆) δ 1.12 (s, 6H), 2.16 (td, J = 12.4, 3.6 Hz, 2H), 2.27 (d, J = 12.6 Hz, 2H), 2.46 (d, J = 11.5 Hz, 2H), 2.86 (d, J = 12.4 Hz, 2H), 3.21 (s, 3H), 3.22- 3.26 (m, 2H), 3.33 (t, J = 5.8 Hz, 2), 7.29-7.38 (m, 2H), 7.50-7.58 (m, 1H), 7.77 (t, J = 6.0 Hz, 1H). |
| 126 | 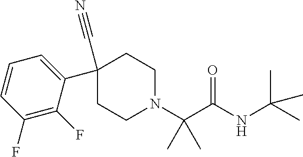 [SMILES: CC(C)(C)NC(=O)C(C)(C)N1CCC(C#N)(c2cccc(F)c2F)CC1] | 364 | 2.0 | (DMSO-d₆) δ 1.11 (s, 6H), 1.25 (s, 9H), 2.09 (td, J = 12.5, 3.5 Hz, 2H), 2.31 (d, J = 12.8 Hz, 2H), 2.43-2.49 (m, 2H), 2.85 (d, J = 12.2 Hz, 2H), 7.11 (s, 1H), 7.29-7.40 (m, 2H), 7.50-7.59 (m, 1H). |
| 127 | 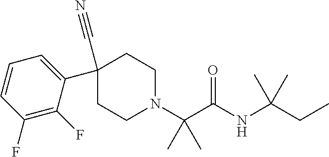 [SMILES: CCC(C)(C)NC(=O)C(C)(C)N1CCC(C#N)(c2cccc(F)c2F)CC1] | 378 | 1.5 | (DMSO-d₆) δ 0.76 (t, J = 7.4 Hz, 3H), 1.11 (s, 6H), 1.20 (s, 6H), 1.64 (q, J = 7.5 Hz, 2H), 2.09 (td, J = 12.5, 3.5 Hz, 2H), 2.31 (d, J = 12.7 Hz, 2H), 2.47 (d, J = 12.7 Hz, 2H), 2.87 (d, J = 12.1 Hz, 2H), 7.04 (s, 1H), 7.29-7.40 (m, 2H), 7.49-7.58 (m, 1H). |
| 128 | 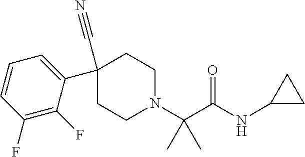 [SMILES: CC(C)(C(=O)NC1CC1)N1CCC(C#N)(c2cccc(F)c2F)CC1] | 348 | 1.7 | (DMSO-d₆) δ 0.42-0.48 (m, 2H), 0.56- 0.62 (m, 2H), 1.10 (s, 6H), 2.13 (td, J = 12.4, 3.6 Hz, 2H), 2.25 (d, J = 12.1 Hz, 2H), 2.45 (t, J = 11.8 Hz, 2H), 2.56-2.65 (m, 1H), 2.80 (d, J = 12.1 Hz, 2H), 7.29-7.37 (m, 2H), 7.48-7.58 (m, 1H), 7.63 (d, J = 4.4 Hz, 1H). |
| 129 | 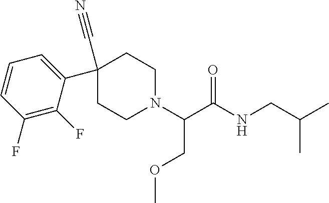 [SMILES: COCC(C(=O)NCC(C)C)N1CCC(C#N)(c2cccc(F)c2F)CC1] | 380 | 4.3 | (DMSO-d₆) δ 0.84 (d, J = 6.6 Hz, 6H), 1.71 (hept, J = 6.7 Hz, 1H), 1.99-2.15 (m, 2H), 2.16-2.28 (m, 2H), 2.68 (t, J = 11.9 Hz, 1H), 2.75-2.87 (m, 2H), 2.88-3.06 (m, 3H), 3.24 (s, 3H), 3.30 (d, J = 6.0 Hz, 1H), 3.56 (dd, J = 9.9, 6.3 Hz, 1H), 3.68 (dd, J = 9.9, 6.0 Hz, 1H), 7.27-7.36 (m, 2H), 7.47-7.57 (m, 1H), 7.96 (t, J = 6.0 Hz, 1H). |

### Biological Examples

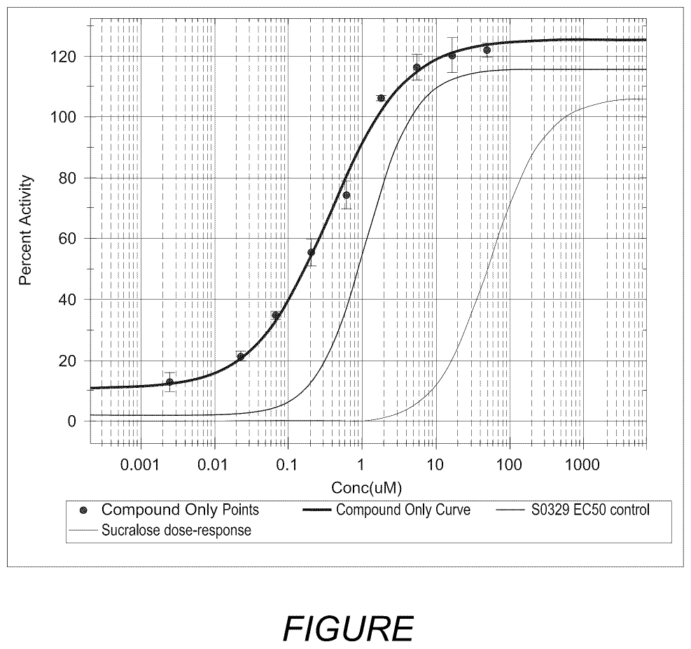

Compounds described in this application show moderate to potent agonist effect on the sweet receptor based assay. Potencies (EC₅₀ values, e.g. see Tables 1-3) for agonist activity were measured against the hT1R2/T1R3 taste receptor. FIG. 1 exemplifies the data generated during the screening to determine EC₅₀ values.

### Sensory Experiments

Compounds described in this application are expected to show potent in vivo ago-potentiating sweet taste effects. Compound 78, for example exhibits remarkable HFCS and Sucrose sweet enhancement effect in taste tests, Table 4.

TABLE 4
Results from sweet enhancement taste tests.

| Study # | Sample | pH | Results | p-Value |
|---|---|---|---|---|
| 1 | 10 ppm Compound 78 | 2.8 | 9 | <0.001 |
| | 1.5% Sucrose | | 42 | |
| 2 | 7 ppm Compound 78 + 6% sucrose | | 21 | 0.383 |
| | 8% Sucrose | | 24 | |
| 3 | 9 ppm Compound 78 + 6% HFCS | | 22 | 0.766 |
| | 8% HFCS | | 23 | |

Test samples containing either a sweetener (sucrose or high-fructose corn syrup (HFCS)) alone or in combination with a test compound were presented in pairs to a group of panelists who were asked to determine which of the samples was sweeter. Subjects refrained from eating or drinking (except water) for at least 1 hour prior to the test. Subjects rinsed with water several times to clean the mouth. The samples within a paired comparison test were presented in a randomized, counterbalanced order. Panelists had up to a 1 minute delay between taste tests to clear the mouth of any tastes. Binomial probability tables were used to determine the probability of the correct number of responses occurring for each test at alpha-0.05.

As shown in Table 4 (test #1), 10 ppm of the compound 78 was found to be significantly less sweet than 1.5% sucrose at pH 2.8. However, when a sample containing 10 ppm of compound 78+6% sucrose was compared with 8% sucrose (test #2), no significant difference in sweetness was detected between the two samples. Similar results were also obtained when a sample containing 10 ppm of compound 78+6% HFCS was compared with 8% HFCS (test #3). Thus, these results indicate that compound 78 is able to achieve significant enhancement of both sucrose and HFCS at pH 2.8 even at concentration far below its sweetness threshold under similar conditions.

The foregoing detailed description has been given for clearness of understanding only and no unnecessary limitations should be understood therefrom as modifications will be obvious to those skilled in the art. It is not an admission that any of the information provided herein is prior art or relevant to the presently claimed embodiments, or that any publication specifically or implicitly referenced is prior art.

1. A compound having the structure of formula (I): 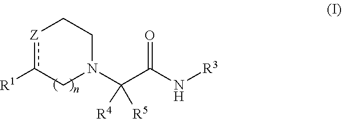
or a salt or stereoisomer thereof, wherein: [symbol]
indicates a single or double bond;
when [symbol] is a single bond, then Z is CR⁶R⁷ or NR⁸;
when [symbol] is a double bond, then Z is CR⁶;
R⁶ is an aryl optionally substituted with one or more R⁹, a heteroaryl optionally substituted with one or more R⁹, or a heterocyclyl optionally substituted with one or more R⁹;
R⁷ is hydrogen, cyano, OR¹⁰, or C₁₋₆ alkyl optionally substituted with one or more R⁹;
or alternatively, R⁶ and R⁷, taken together with the atom to which they are attached, form a heterocyclyl optionally substituted with one or more R⁹;
R¹ is hydrogen, or alternatively, R¹ and R⁶, taken together with the atoms to which they are attached, form a fused aromatic ring and R⁷ is absent;
R³ is C₁₋₆ alkyl optionally substituted with one or more R⁹, cycloalkyl, aralkyl, or (carbocyclyl)alkyl;
R⁴ is hydrogen or a C₁₋₆ alkyl substituted with one or more R⁹;
R⁵ is a C₁₋₆ alkyl substituted with one or more R⁹; or alternatively, R⁴ and R⁵, taken together with the atom to which they are attached, form a heterocyclyl optionally substituted with one or more R⁹;
R⁸ is an aryl substituted with one or more R⁹, or a heteroaryl optionally substituted with one or more R⁹;
n is 1 or 2;
each R⁹ is independently F, Br, I, C-carboxy, C-amido, cycloalkyl, OR¹⁰, cyano, amino, aminoalkyl, C₁-C₆ alkoxy, or C₁₋₆ alkyl optionally substituted with one or more OR¹⁰, C₁-C₆ alkoxy, phenyl, or halogen; or alternatively, two R⁹ groups taken together with the atom(s) to which they are attached, form a heterocyclyl or aryl; and
R¹⁰ is hydrogen or C₁₋₆ alkyl.

2. The compound of claim 1, wherein n is 1.

3. (canceled)

4. The compound of claim 1, wherein R¹ is hydrogen.

5. (canceled)

6. The compound of claim 1, wherein Z is CR⁶R⁷.

7. The compound of claim 1, wherein Z is NR⁸.

8. The compound of claim 1, having the structure of formula (Ia): 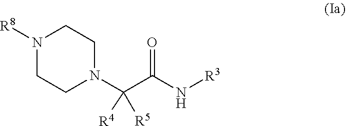
or a salt or stereoisomer thereof.

9. The compound of claim 8 wherein R⁸ is an aryl optionally substituted with one or more R⁹.

10. (canceled)

11. (canceled)

12. (canceled)

13. (canceled)

14. The compound of claim 1, having the structure of formula (Ib): 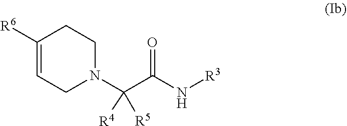
or a salt or stereoisomer thereof.

15. The compound of claim 14, wherein R⁶ is aryl optionally substituted with one or more R⁹.

16. (canceled)

17. (canceled)

18. (canceled)

19. (canceled)

20. (canceled)

21. The compound of claim 1, having the structure of formula (Ic): 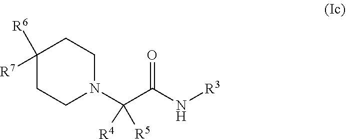
or a salt or stereoisomer thereof.

22. The compound of claim 21, wherein R⁶ is an aryl optionally substituted with one or more R⁹.

23-37. (canceled)

38. The compound of claim 1, wherein R³ is C₁₋₆ alkyl optionally substituted with one or more R⁹.

39. (canceled)

40. (canceled)

41. (canceled)

42. (canceled)

43. The compound of claim 1, wherein R³ is alkyl substituted with one or more OR¹⁰ or amino.

44. The compound of claim 1, wherein R³ is cycloalkyl.

45. (canceled)

46. (canceled)

47. (canceled)

48. (canceled)

49. (canceled)

50. (canceled)

51. The compound of claim 1, wherein R⁴ is hydrogen.

52. The compound of claim 1, wherein R⁴ is C₁₋₆ alkyl optionally substituted with one or more R⁹.

53. (canceled)

54. (canceled)

55. The compound of claim 1, wherein R⁵ is C₁₋₆ alkyl optionally substituted with one or more R⁹.

56. (canceled)

57. (canceled)

58. (canceled)

59. A compound selected from the group consisting of: 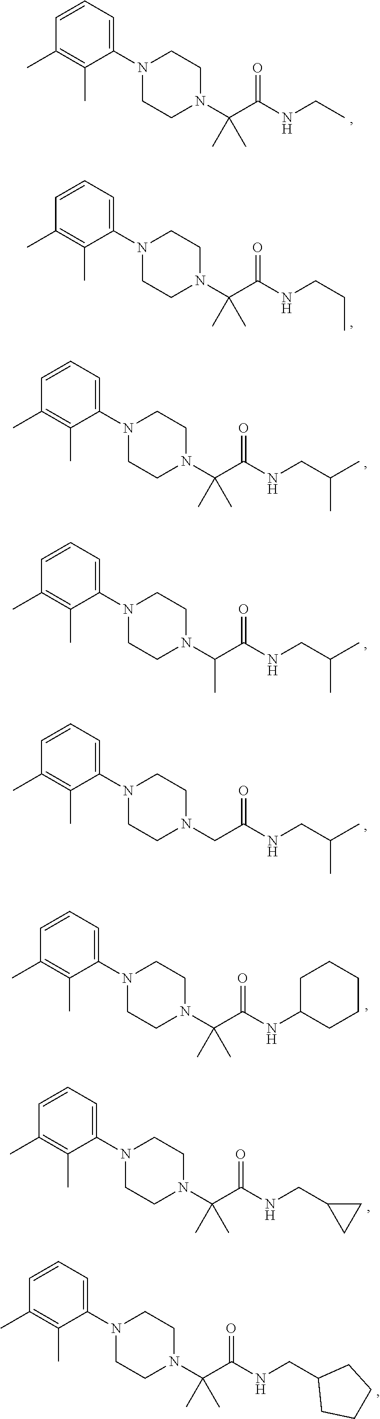 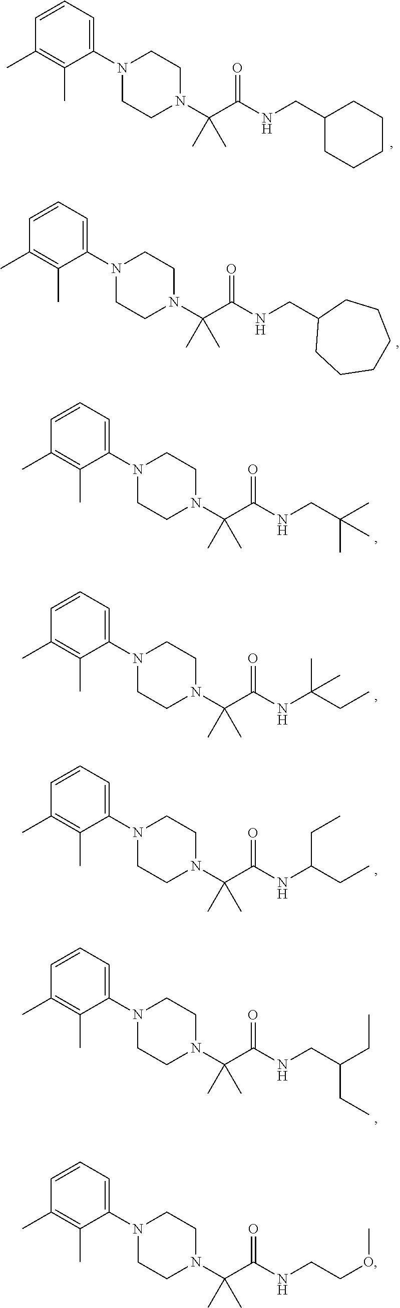 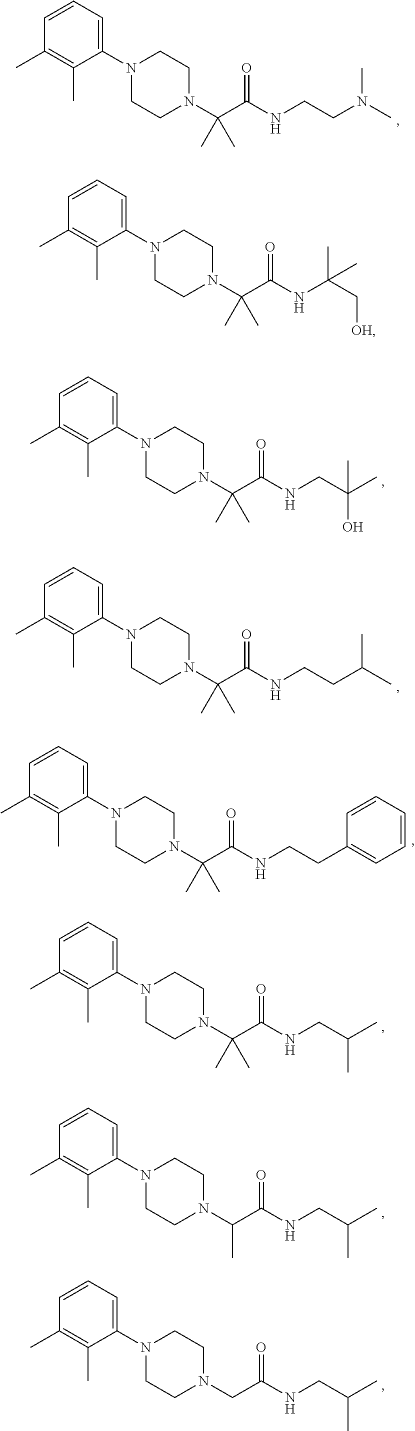 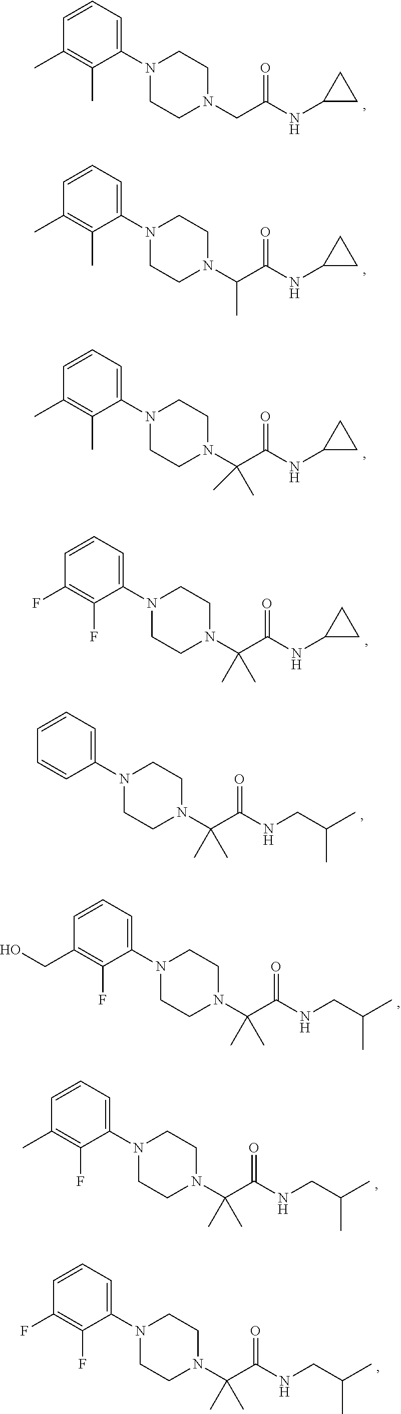 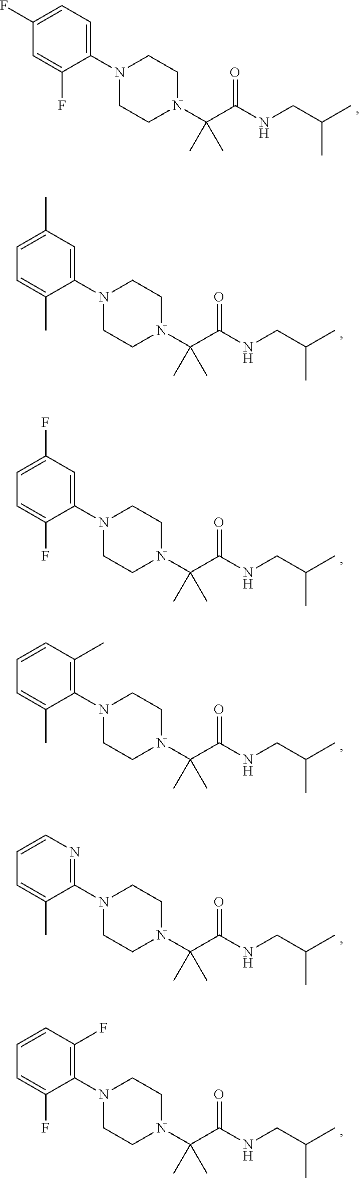 [SMILES: CC(C)CNC(=O)C(C)(C)N1CCN(c2ccc(F)cc2F)CC1 ; Cc1ccc(C)c(N2CCN(C(C)(C)C(=O)NCC(C)C)CC2)c1 ; CC(C)CNC(=O)C(C)(C)N1CCN(c2cc(F)ccc2F)CC1 ; Cc1cccc(C)c1N1CCN(C(C)(C)C(=O)NCC(C)C)CC1 ; Cc1cccnc1N1CCN(C(C)(C)C(=O)NCC(C)C)CC1 ; CC(C)CNC(=O)C(C)(C)N1CCN(c2c(F)cccc2F)CC1] 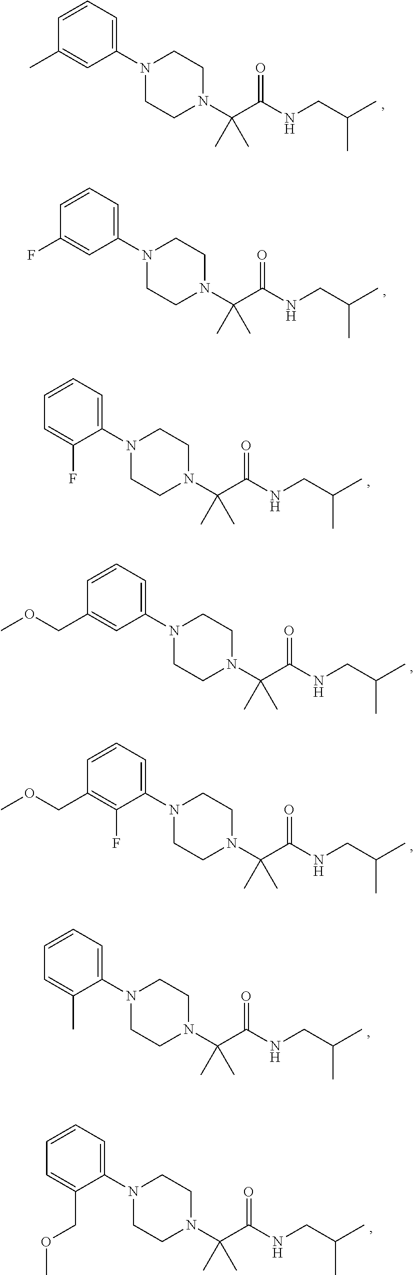 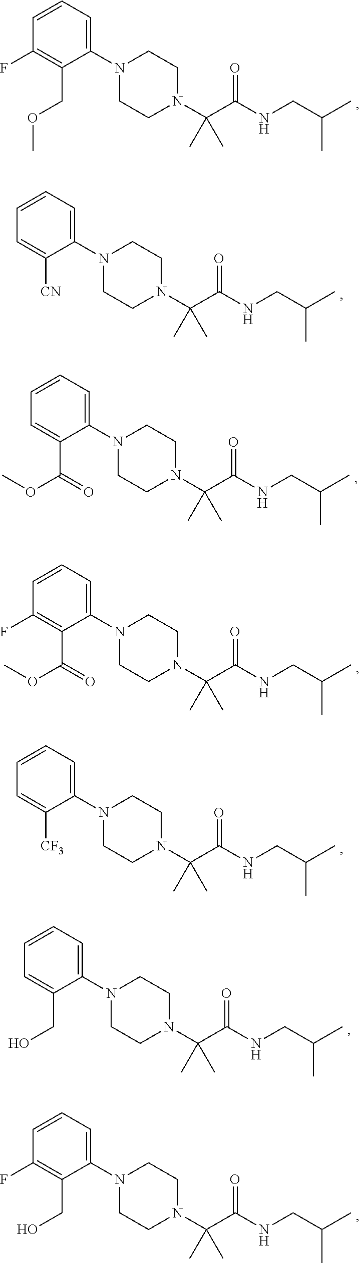 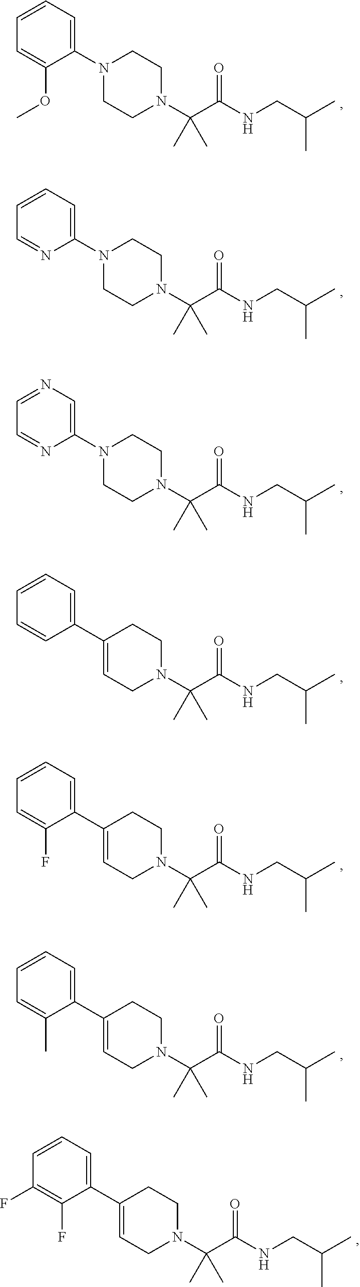 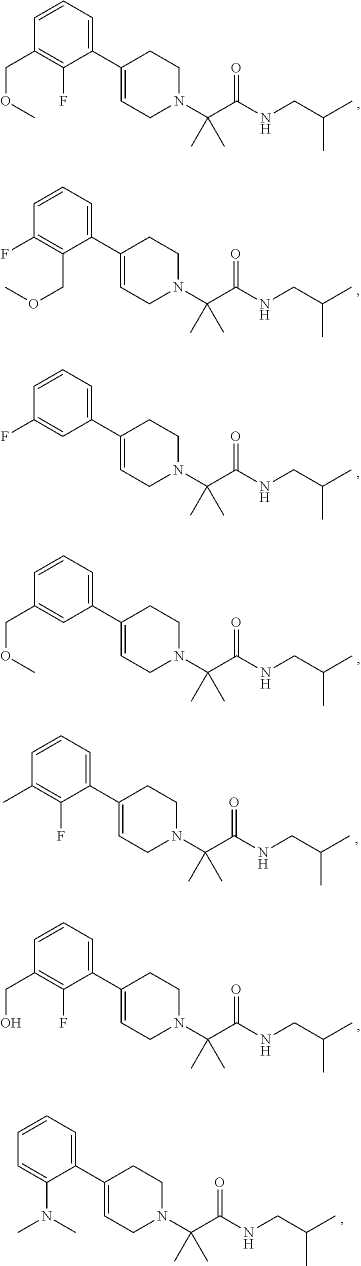 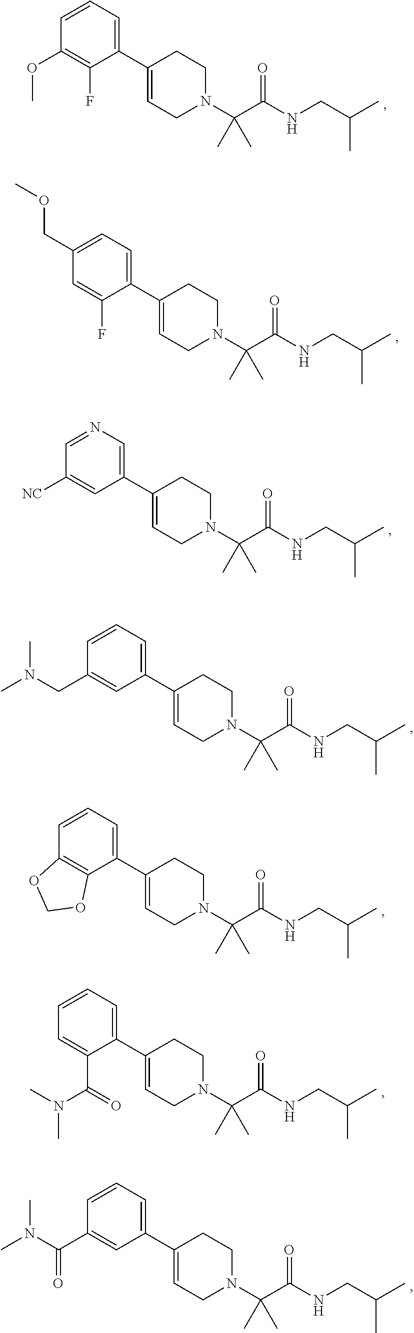 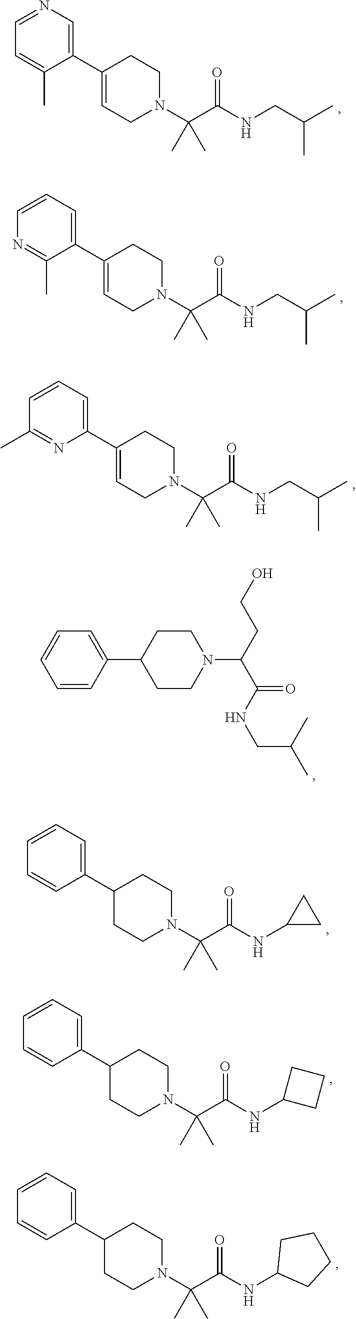 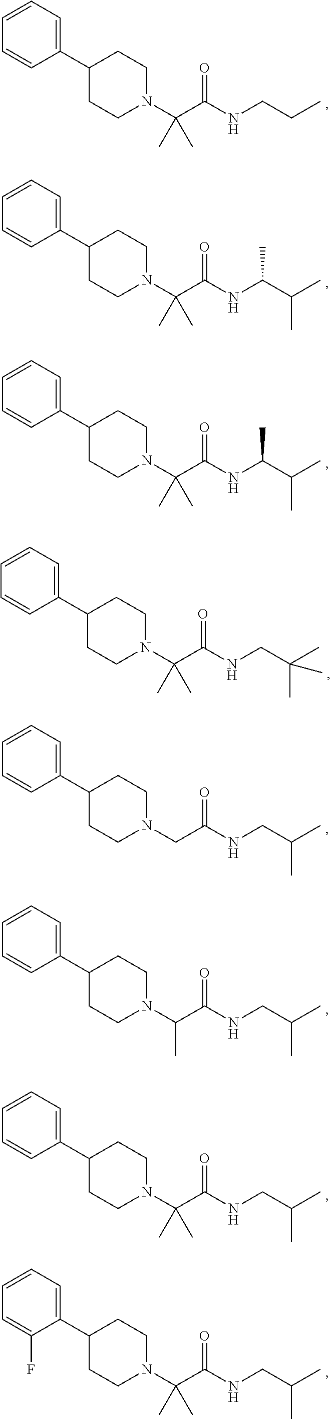 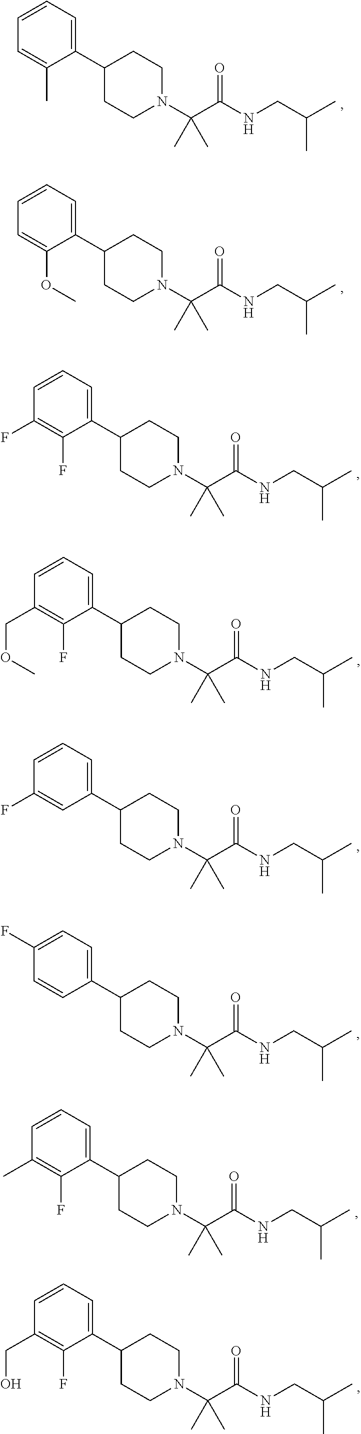 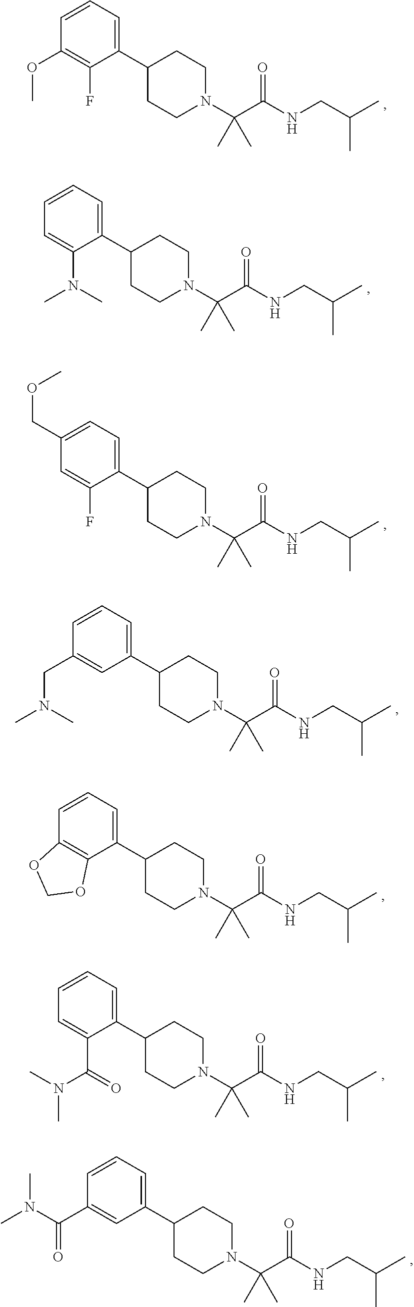 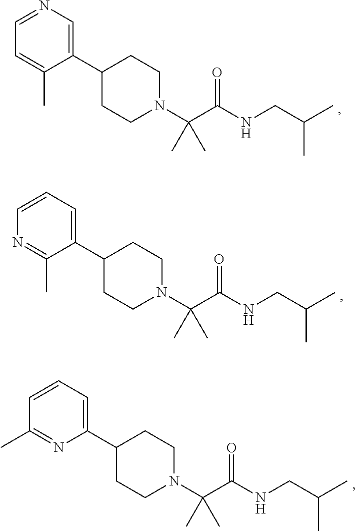 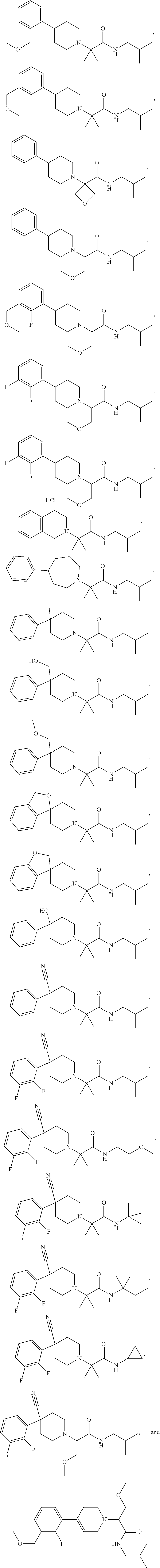
or a salt or stereoisomer thereof.

60. An ingestible composition comprising the compound of claim 1 and one or more sweeteners.

61. (canceled)

62. (canceled)

63. (canceled)

64. (canceled)

65. (canceled)

66. A method of enhancing sweetness of a sweetener, comprising combining the compound of claim 1 with the sweetener.

67-73. (canceled)
